(19) United States
(12) Patent Application Publication
(10) Pub. No.: US 2012/0214830 A1
(43) Pub. Date: Aug. 23, 2012

(54) BIOLOGICAL MARKERS PREDICTIVE OF ANTI-CANCER RESPONSE TO INSULIN-LIKE GROWTH FACTOR-1 RECEPTOR KINASE INHIBITORS IN HEPATOCELLULAR CARCINOMA

(75) Inventors: Elizabeth A. Buck, Huntington, NY (US); Hui Zhao, Farmingdale, NY (US)

(73) Assignee: OSI Pharmaceuticals, LLC

(21) Appl. No.: 13/402,147

(22) Filed: Feb. 22, 2012

Related U.S. Application Data
(60) Provisional application No. 61/463,696, filed on Feb. 22, 2011.

(51) Int. Cl. A61K 31/517; G01N 33/566; A61P 35/00; C40B 30/04

(52) U.S. Cl. 514/266.4; 506/9; 436/501

(57) ABSTRACT

The present invention provides diagnostic methods for predicting the effectiveness of treatment of an hepatocellular carcinoma (HCC) patient with an IGF-1R kinase inhibitor by assessing whether the HCC tumor cells express a high level of AFP, or whether serum levels of AFP protein are high. The present invention also provides diagnostic methods for predicting the effectiveness of treatment of cancer patients with IGF-1R kinase inhibitors, based on a determination of the expression level of IR, IGF-2, IGFBP3 or IGFBP7 in tumor cells, or a 4-gene index calculated using the expression vales for each of these four genes, which can be used to identify tumors that will be sensitive to IGF-1R kinase inhibitors, and also those that will be insensitive. Improved methods for treating cancer patients with IGF-1R kinase inhibitors that incorporate the above methods are also provided.

### CROSS-REFERENCE TO RELATED APPLICATIONS

This application claims the benefit of U.S. Provisional Application No. 61/463,696, filed Feb. 22, 2011, which is herein incorporated by reference in its entirety.

### BACKGROUND OF THE INVENTION

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body. The present invention is directed to methods for diagnosing and treating cancer patients. In particular, the present invention is directed to methods for determining which patients will most benefit from treatment with an insulin-like growth factor-1 receptor (IGF-1R) kinase inhibitor.

IGF-1R belongs to the insulin receptor family that includes the Insulin Receptor (IR), IGF-1R (homodimer), IGF-1R/IR (hybrid receptor), and IGF-2R (mannose 6-phosphate receptor). IGF-1R/IR hybrids act as homodimers, preferentially binding and signaling with IGFs. IR exists in two isoforms: IR-B (traditional insulin receptor) and IR-A (a fetal form which is re-expressed in selected tumors and preferentially binds IGF-II). IGF-2R is a non-signaling receptor that acts as a “sink” for IGF-II (Pollak M. N., et al. Nat Rev Cancer 2004 4:505-18). Six well-characterized insulin-like growth factor binding proteins (IGFBP-1 through -6) associate with IGF ligands to stabilize the IGFs and modulate their ability to bind the IGF-IR.

IGF-1R is a transmembrane RTK that binds primarily to IGF-1 but also to IGF-II and insulin with lower affinity. Binding of IGF-1 to its receptor results activation of receptor tyrosine kinase activity, intermolecular receptor autophosphorylation and phosphorylation of cellular substrates (major substrates are IRS1 and Shc). The ligand-activated IGF-1R induces mitogenic activity in normal cells and plays an important role in abnormal growth. A major physiological role of the IGF-1 system is the promotion of normal growth and regeneration. Overexpressed IGF-1R (type 1 insulin-like growth factor receptor) can initiate mitogenesis and promote ligand-dependent neoplastic transformation. Furthermore, IGF-1R plays an important role in the establishment and maintenance of the malignant phenotype. Unlike the epidermal growth factor (EGF) receptor, no mutant oncogenic forms of the IGF-1R have been identified. However, several oncogenes have been demonstrated to affect IGF-1 and IGF-1R expression. The correlation between a reduction of IGF-1R expression and resistance to transformation has been seen. Exposure of cells to the mRNA antisense to IGF-1R RNA prevents soft agar growth of several human tumor cell lines. IGF-1R abrogates progression into apoptosis, both in vivo and in vitro. It has also been shown that a decrease in the level of IGF-1R below wild-type levels causes apoptosis of tumor cells in vivo. The ability of IGF-1R disruption to cause apoptosis appears to be diminished in normal, non-tumorigenic cells.

The IGF-1 pathway has an important role in human tumor development. IGF-1R overexpression is frequently found in various tumors (breast, colon, lung, sarcoma) and is often associated with an aggressive phenotype. High circulating IGF1 concentrations are strongly correlated with prostate, lung and breast cancer risk. Furthermore, IGF-1R is required for establishment and maintenance of the transformed phenotype in vitro and in vivo (Baserga R. Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models. The IGF-1R signaling pathway also appears to be a robust target in colorectal cancer (CRC), based upon data demonstrating overexpression of the receptor and ligands in CRC, association with a more malignant phenotype, chemotherapy resistance, and correlation with a poor prognosis (Saltz, L. B., et al. J Clin Oncol 2007; 25(30): 4793-4799; Tripkovic I., et al. Med. Res. 2007 July; 38(5):519-25. Epub 2007 Apr. 26; Miyamoto S., et al. Clin Cancer Res. 2005 May 1; 11(9):3494-502; Nakamura M., et al. Clin Cancer Res. 2004 Dec. 15; 10(24):8434-41; Grothey A, et al. J Cancer Res Clin Oncol. 1999; 125(3-4):166-73).

It has been recognized that inhibitors of protein-tyrosine kinases are useful as selective inhibitors of the growth of mammalian cancer cells. For example, GLEEVEC™ (also known as imatinib mesylate), a 2-phenylpyrimidine tyrosine kinase inhibitor that inhibits the kinase activity of the BCR-ABL fusion gene product, has been approved by the U.S. Food and Drug Administration for the treatment of CML. The 4-anilinoquinazoline compound TARCEVA™ (erlotinib HCl) has also been approved by the FDA, and selectively inhibits EGF receptor kinase with high potency. The development for use as anti-tumor agents of compounds that directly inhibit the kinase activity of IGF-1R, as well as antibodies that reduce IGF-1R kinase activity by blocking IGF-1R activation or antisense oligonucleotides that block IGF-1R expression, are areas of intense research effort (e.g. see Larsson, O. et al (2005) Brit. J. Cancer 92:2097-2101; Ibrahim, Y. H. and Yee, D. (2005) Clin. Cancer Res. 11:944s-950s; Mitsiades, C. S. et al. (2004) Cancer Cell 5:221-230; Camirand, A. et al. (2005) Breast Cancer Research 7:R570-R579 (DOI 10.1186/bcr1028); Camirand, A. and Pollak, M. (2004) Brit. J. Cancer 90:1825-1829; Garcia-Echeverria, C. et al. (2004) Cancer Cell 5:231-239; Sachdev D, and Yee D., Mol Cancer Ther. 2007 Jan.; 6(1):1-12; Hofmann F., and Garcia-Echeverria C., Drug Discov Today 2005 10:1041-7). Agents inhibiting the IGF-1R pathway have demonstrated anti-tumor efficacy in multiple human cancer models both in vitro and in vivo, particularly in pediatric models of Ewing's sarcoma and rhabdomyosarcoma (Manara M C, et al. Int J Oncol 2005 27:1605-16). Despite early hints of efficacy in patients with sarcoma, results to date of IGF-1R inhibitors in early clinical trials have not been impressive, indicating that patient selection strategies and rational combinations may be needed to move forward with this approach (Tolcher A. W., et al. Journal of Clinical Oncology, 2007 ASCO Annual Meeting Proceedings Part I. Vol 25, No. 18S (June 20 Supplement), 2007: 3002). Data acquired thus far, has not indicated that activation, overexpression, or amplification of members of the IGF-1R pathway will predict responsiveness.

IGF-1R/IR signaling can mediate activation of cellular survival in the presence of a multitude of other anti-tumor agents including cytotoxic chemotherapeutics and radiation as well as molecular targeted therapies (MTTs). The ability for IGF-1R/IR inhibitors to augment the efficacy for these agents has been extensively investigated in the preclinical setting and is currently being actively persued in the clinical setting. Resistance to both radiation and cytotoxic chemotherapies can be associated with increased activity through the AKT survival pathway, which can be driven by IGF-1R/IR signaling. Radiation treatment achieves augmented anti-tumor activity upon co-administration of an IGF-1R antagonist in in vivo xenograft models. In numerous settings IGF-1R inhibitors have been shown to augment the cytotoxic effects for chemotherapies including paclitaxel and doxorubicin (Wang, Y. H. et al., Mol. Cell. Biochem., 2009, 327, 257; Allen, G. W. et al. Cancer Res., 2007, 67, 1155; Zeng, X., et al. Clin. Cancer Res., 2009, 15, 2840; Martins, A. S. et al. Clin. Cancer Res., 2006, 12, 3532). Similar to observations with radiation, tumor cells can also upregulate AKT survival signaling in response to cytotoxic chemotherapies. Recent studies have shown that cytotoxic agents including paclitaxel can evoke specific upregulation of IGF-1R activity, and IGF-1R inhibitors can augment the pro-apoptotic potential for such agents (P. Chinnaiyan, G. W. et al., (2006) Semin. Radiat. Oncol., 16, 59-64). These preclinical data have provided strong rationale for a multitude of clinical studies evaluating IGF-1R inhibitors in combination with chemotherapeutics.

Several groups have investigated or disclosed potential biomarkers to predict a patient's response to protein-tyrosine kinase inhibitors (see for example, PCT publications: WO 2004/063709, WO 2005/017493, WO 2004/111273, WO 2008/108986, WO 2007/001868, WO 2006/045991, WO 2010/048304 and WO 2004/071572; U.S. Pat. No. 7,794,960; US published patent applications: US 2005/0019785, US 2007/0065858, US 2009/0092596, US 2009/0093488, US 2006/0140960 and US 2004/0132097; Brugger, W. et al. (2009) J Clin Oncol 27:15s, (suppl; abstr 8020); Siena, S et al (2009) JNCI 101(19):1308-1324; Riely and Ladanyi (2008) J Mol Diagnostics 10(6):493; Jimeno, A. et al. (2009) Cancer J. 15(2):110-13; and Fuchs, B C et al., (2008) Cancer Res. 68(7):2391-2399). In addition, several biomarkers have been disclosed that have potential in predicting a patient's response to IGF-1R kinase inhibitors, (e.g. see Rodon, J. et al (2008) Mol Cancer Ther. 7:2575-2588; T. Pitts et al. (2009) EORTC Conference, Boston, Mass., abstract #2141; Huang, F. et al. (2009) Cancer Res. 69(1):161-170; Rodon, J. et al., (2008) Mol. Cancer. Ther. 7:2575-2588; U.S. Pat. No. 7,811,562; US patent application publications US 2010/0184125, US 2010/0240665, US 2010/0166747, US 2008/0112888 and US 2010/0316639; Pitts T. M. et al, (2010) Clin Can Res. 16(12) 3193-3204; Yee, D., (2010) Clin Can Res. 16(12) 3091-3093; International patent publications WO 2009/079587, WO 2010/048123, WO 2010/119126 and WO 2008/144345). However, in most instances no FDA-approved diagnostic tests have yet emerged that can effectively guide practicing physicians in the treatment of their patients with such inhibitors, or can indicate to the physician which tumors will respond most favorable to a combination of such an inhibitor with a standard chemotherapy agent.

Hepatocellular carcinoma (HCC) is the 5^{th} most common cancer and the 3^{rd} most important cause of cancer mortality. HCC develops on a background of a chronically diseased liver caused by various factors, such as alcohol, or HBV and HCV infections. Current methods for diagnosis and screening include physical examination, ultrasound imaging and serum alpha-fetoprotein (AFP) concentration measurement (Debruyne E N, et al. Clinica chimica acta 2008, 395(1-2):19-26). Based on gene expression signatures, HCC can be categorized into several subgroups with distinct clinical features (Chiang, D. Y. et al. (2008) Cancer Res. 68:6779-6788 (including supplementary online data); Hoshida Y, et al. Cancer Res 2009, 69(18):7385-92; Tovar V, et al. (2010) J Hepatol; 52(4):550-9), termed WNT-CTIVNB1, Proliferation, IFN-response, polysomy of chromosome 7, and unannotated subclasses.

Alpha-fetoprotein (AFP) is a 70 kDa glycoprotein synthesized by the yolk sac during early fetal life and later on by the fetal liver (Debruyne E N, et al. Clinica chimica acta 2008; 395(1-2):19-26). Its synthesis peaks at 12-16 weeks of gestation, and drops rapidly after birth. Serum AFP concentration is below 10 ng/ml in adults under normal conditions. The biological functions of AFP are not clear. It has been proposed that AFP could function as a chaperone for other molecules (e.g. bilirubin, fatty acids, etc); acts as an immunosuppressive agent; and that it plays a role in regulation of cell growth.

Elevated AFP serum levels have been principally used as a tumor marker for HCC (Debruyne, E. N. et al. (2008) Clinica Chimica Acta 395: 19-26), although it is pathologically associated with several other carcinoma types (e.g. gastric, lung, biliary tract, and testicular cancer). Specificity and sensitivity of AFP as a diagnostic marker varies depending on the cut-off values used. A cut-off value of 100 ng/ml has about 99% specificity, but only 30% sensitivity for detecting HCC. Serum AFP concentrations in HCC patients are influenced by the tumor size and differentiation status. 80% of the cases of small HCC showed no increase in AFP concentration. Two subgroups of HCC with a median tumor size of less than 3 cm had a median AFP concentration of less than 50 ng/ml, compared to 170 ng/ml for a subgroup with median tumor size of 4.5 cm (Hoshida Y, et al. Cancer Res 2009, 69(18):7385-92). Patients with well-differentiated HCC tumors tend to have lower serum AFP concentration.

In addition to a diagnostic tool, AFP is also used to monitor the response to treatment in HCC patients with elevated AFP. Decrease in serum AFP concentration with a half-time of less than 5 days and a normalization to an AFP serum concentration to <10 ng/ml within 30 days is one criterion to assess treatment effectiveness (Debruyne E. N., et al. Clinica chimica acta 2008; 395(1-2):19-26).

AFP has three isoforms (L1, L2 and L3) based on differences in their carbohydrate moiety, and their respective binding affinities toward Lens culinaris agglutinin (LCA) lectin (Debruyne E. N., et al. Clinica chimica acta 2008, 395(1-2):19-26; L1 D, et al. Clinica chimica acta; 2001, 313(1-2):15-19). AFP-L1 is present in chronic hepatitis and liver cirrhosis, and constitutes a majority fraction of total AFP in the non-malignant liver diseases. AFP-L3 appears to be produced only by cancer cells, and is a marker of biologic malignancy of HCC. AFP-L3 to total AFP ratio may be an indicator of tumor aggressiveness (i.e. AFP-L3 positive HCC has the potential for rapid growth and early distant metastasis). The relationship between AFP-L3 positive HCC and well-differentiated or mesenchymal HCC is not clear.

Bioavailability of both IGFs and insulin is influenced by the presence of secreted IGFBPs (Murphy L J. Journal of molecular endocrinology 1998; 21(2):97-107). IGFBPs are a superfamily of homologous proteins with a total of 15 members. They can be isolated from serum, other biological fluids, and tissue extracts. They vary in molecular size, hormonal control, and functional significance. All members of the proposed IGFBP superfamily preserve an N-terminal cysteine-rich domain, including the IGFBP motif GCGCCXXC, but vary in the intermediate region and C-terminal domain of the protein. IGFBP1 to 6 have high affinity for IGFs, whereas IGFBP7 shows a much higher affinity for insulin (Yamanaka Y, et al. J. Biol. Chem. 1997; 272(49):30729-34).

During most cancer metastases, an important change occurs in a tumor cell known as the epithelial-mesenchymal transition (EMT) (Thiery, J. P. (2002) Nat. Rev. Cancer 2:442-454; Savagner, P. (2001) Bioessays 23:912-923; Kang Y. and Massague, J. (2004) Cell 118:277-279; Julien-Grille, S., et al. Cancer Research 63:2172-2178; Bates, R. C. et al. (2003) Current Biology 13:1721-1727; Lu Z., et al. (2003) Cancer Cell. 4(6):499-515)). EMT does not normally occur in healthy cells except during embryogenesis, though a transient EMT state is induced in epithelial wound healing to aid in the reconstruction of epithelial tissue. Epithelial cells, which are bound together tightly and exhibit polarity, change to a more mesenchymal cellular phenotype, in which these mesenchymal cells are held together more loosely, exhibit a loss of polarity, and have the ability to move within tissues. These mesenchymal-like cells can spread into tissues surrounding the original tumor, as well as separate from the tumor, invade blood and lymph vessels, and travel to new locations where they divide and form additional tumors. Recent research has demonstrated that some epithelial cells respond well to EGFR and insulin-like growth factor-1 receptor (IGF-1R) kinase inhibitors, but that after an EMT the resulting mesenchymal-like tumor cells are much less sensitive to such inhibitors. (e.g. see Thompson, S. et al. (2005) Cancer Res. 65(20):9455-9462; US 2007/0065858; US 20090092596; U.S. Patent Application 60/997,514). Thus there is a pressing need for anti-cancer agents that can prevent or reverse tumor cell EMT events (e.g. stimulate a mesenchymal to epithelial transition (MET)), or inhibit the growth of the mesenchymal-like tumor cells resulting from EMT. Such agents should be particularly useful when used in conjunction with other anti-cancer drugs such as EGFR and IGF-1R kinase inhibitors.

As human cancers progress to a more invasive, metastatic state, multiple signaling programs regulating cell survival and migration are observed depending on cell and tissue contexts (Gupta, G. P., and Massague, J. (2006) Cell 127, 679-695). Recent data highlight the transdifferentiation of epithelial cancer cells to a more mesenchymal-like state, a process resembling epithelial-mesenchymal transition (EMT; (Oft, M., et al. (1996). Genes & development 10, 2462-2477; Perl, A. K., et al. (1998). Nature 392, 190-193), to facilitate cell invasion and metastasis (Brabletz, T. et al. (2005) Nat Rev Cancer 5, 744-749; Christofori, G. (2006) Nature 441, 444-450). Through EMT-like transitions mesenchymal-like tumor cells are thought to gain migratory capacity at the expense of proliferative potential. A mesenchymal-epithelial transition (MET) has been postulated to regenerate a more proliferative state and allow macrometastases resembling the primary tumor to form at distant sites (Thiery, J. P. (2002) Nat Rev Cancer 2, 442-454). EMT-like transitions in tumor cells result from transcriptional reprogramming over considerable periods of time (weeks to months) via transcription factors harboring zinc finger, forkhead, bHLH and HMG-box domains (Mani, S. A. et al. (2007) Proceedings of the National Academy of Sciences of the United States of America 104, 10069-10074; Peinado, H. et al. (2007) Nat Rev Cancer 7, 415-428). The loss of E-cadherin and transition to a more mesenchymal-like state, with increased expression of mesenchymal proteins such as vimentin or fibronectin, likely serves a major role in the progression of cancer (Matsumura, T. et al. (2001) Clin Cancer Res 7, 594-599; Yoshiura, K. et al. (1995). Proceedings of the National Academy of Sciences of the United States of America 92, 7416-7419) and the acquisition of a mesenchymal phenotype has been correlated with poor prognosis (Baumgart, E. et al. (2007) Clin Cancer Res 13, 1685-1694; Kokkinos, M. I. Et al. (2007) Cells, tissues, organs 185, 191-203; Willipinski-Stapelfeldt, B. et al. (2005) Clin Cancer Res 11, 8006-8014.). Targeting tumor-derived and/or tumor-associated stromal cells provides a unique mechanism to block EMT-like transitions and inhibit the survival of invading cells.

The cellular changes associated with EMT-like transitions alter the dependence of carcinoma cells on EGFR signaling networks for survival. It has been observed that an EMT-like transition was associated with cellular insensitivity to the EGFR kinase inhibitor erlotinib (Thomson, S. et al. (2005) Cancer Research 65, 9455-9462; Witta, S. E., et al. (2006) Cancer Research 66, 944-950; Yauch, R. L., et al. (2005) Clin Cancer Res 11, 8686-8698), in part from EGFR independent activation of either or both the PI3-kinase or Mek-Erk pathways (Buck, E. et al. (2007). Molecular Cancer Therapeutics 6, 532-541). Similar data correlating EMT status to sensitivity to EGFR kinase inhibitors have been reported in pancreatic, CRC (Buck, E. et al. (2007) Molecular Cancer Therapeutics 6, 532-541) bladder (Shrader, M. et al. (2007) Molecular Cancer Therapeutics 6, 277-285) and HNSCC (Frederick et al. (2007) Molecular Cancer Therapeutics 6, 1683-1691) cell lines, xenografts and in patients (Yauch, R. L., et al. (2005) Clin Cancer Res 11, 8686-8698). The molecular determinants to alternative routes of activation of the PI3-kinase and Erk pathways, which can bypass cellular sensitivity to EGFR inhibitors, have been actively investigated (Chakravarti, A. et al. (2002) Cancer research 62, 200-207; Engelman, J. A. et al. (2007) Science 316:1039-1043).

There remains a critical need for improved methods for determining the best mode of treatment for any given cancer patient. The present invention provides new biomarker methods for determining which tumors will respond most effectively to treatment with IGF-1R kinase inhibitors, and for the incorporation of such a determination into more effective treatment regimens for HCC cancer patients with IGF-1R kinase inhibitors, including small-molecule IGF-1R kinase inhibitors, such as OSI-906, or anti-IGF-1R antibodies.

### SUMMARY OF THE INVENTION

The present invention provides new diagnostic methods using gene biomarkers for predicting the effectiveness of treatment of cancer patients with IGF-1R kinase inhibitors, and improved methods for treating cancer patients with IGF-1R kinase inhibitors that utilize said diagnostic methods prior to the administration of a drug.

The present invention provides diagnostic methods for predicting the effectiveness of treatment of a hepatocellular carcinoma (HCC) patient with an IGF-1R kinase inhibitor. These methods are based on the surprising discovery that the sensitivity of hepatocellular carcinoma cell growth to inhibition by IGF-1R kinase inhibitors is predicted by whether such tumor cells express a high level of AFP, wherein tumor cells that possess the latter are more sensitive to inhibition than tumor cells that have a low expression level of AFP. The present invention also provides a method of identifying patients with hepatocellular carcinoma (HCC) who are most likely to benefit from treatment with an IGF-1R kinase inhibitor, comprising: determining the level of AFP protein in the serum of a patient; and identifying the patient as likely to benefit from treatment with an IGF-1R kinase inhibitor if the serum contains a high level of AFP.

Improved methods for treating hepatocellular carcinoma patients with IGF-1R kinase inhibitors that incorporate the above methodology are also provided. Thus, the present invention further provides a method for treating hepatocellular carcinomas in a patient, comprising the steps of diagnosing a patient's likely responsiveness to an IGF-1R kinase inhibitor by assessing whether the tumor cells express a high level of AFP, or the patient has a high serum AFP level, and administering to said patient a therapeutically effective amount of an IGF-1R kinase inhibitor (e.g. OSI-906) if the tumor cells express a high level of AFP, or if the patient has a high serum AFP level.

The present invention also provides diagnostic methods for predicting the effectiveness of treatment of a hepatocellular carcinoma (HCC) patient with an IGF-1R kinase inhibitor, based on data that shows that the EMT status of HCC tumor cells determines their sensitivity to growth inhibition by an IGF-1R kinase inhibitor. Thus, the degree of sensitivity of the HCC tumor cells to an IGF-1R kinase inhibitor can be assessed by determining the degree of expression of an epithelial biomarker in the tumor cells, such that high expression is indicative that the cells are likely to have high sensitivity to growth inhibition by an IGF-1R kinase inhibitor, or conversely, low expresion is indicative that the cells are likely to have have low sensitivity, or be relatively resistant, to growth inhibition by an IGF-1R kinase inhibitor. Similarly, the degree of sensitivity of the HCC tumor cells to an IGF-1R kinase inhibitor can be assessed by determining the degree of expression of a mesenchymal biomarker in the tumor cells, such that low expression is indicative that the cells are likely to have high sensitivity to growth inhibition by an IGF-1R kinase inhibitor, or conversely, high expresion is indicative that the cells are likely to have have low sensitivity, or be relatively resistant, to growth inhibition by an IGF-1R kinase inhibitor. Improved methods for treating hepatocellular carcinoma patients with IGF-1R kinase inhibitors that incorporate the above methodology are also provided.

The present invention also provides diagnostic methods for predicting the effectiveness of treatment of a hepatocellular carcinoma (HCC) patient with an IGF-1R kinase inhibitor based on the discovery that the degree of sensitivity of HCC tumor cell growth to an IGF-1R kinase inhibitor can be assessed by determining the degree of expression of INSR, IGF-2, IGFBP3 or IGFBP7 in the HCC tumor cells. High expression of INSR or IGF-2 is indicative that the cells are likely to have high sensitivity to growth inhibition by an IGF-1R kinase inhibitor, or conversely, low expresion of INSR or IGF-2 is indicative that the cells are likely to have have low sensitivity, or be relatively resistant, to growth inhibition by an IGF-1R kinase inhibitor. High expression of IGFBP3 or IGFBP7 is indicative that the cells are likely to have low sensitivity to growth inhibition by an IGF-1R kinase inhibitor, or conversely, low expresion of IGFBP3 or IGFBP7 is indicative that the cells are likely to have have high sensitivity, or be relatively resistant, to growth inhibition by an IGF-1R kinase inhibitor. A 4-gene index score calculated using the HCC expression values for each of these four genes was also found to significantly correlate with sensitivity of HCC tumor cells to an IGF-1R kinase inhibitor, and to a much greater degree than any individual gene expression values. These observations provide the basis for additional diagnostic methods for predicting the effects of IGF-1R kinase inhibitors on HCC tumor growth, giving oncologists additional biomarkers to assist them in choosing the most appropriate treatment for their patients. These diagnostic methods involving determining the degree of expression of one or more of INSR, IGF-2, IGFBP3 and IGFBP7 are also expected to be useful for cancers other than HCC. They may also be included as apart of a method of treatment regimen prior to the administration of an IGF-1R kinase inhibitor.

### BRIEF DESCRIPTION OF THE FIGURES

FIG. 1: Sensitivity of HCC cell lines to OSI-906. Twenty-one HCC cell lines were treated with serial dilutions of OSI-906 for 72 hours, and proliferation assays were performed using a CELLTITER-GLO® kit (Promega, Madison, Wis.). The top panel contains HCC cell lines from ATCC, and the bottom panel contains HCC cell lines from Japan Cell Bank, each as indicated by the legend.

FIG. 2: AFP gene expression in HCC cell lines. Total RNA was isolated from 21 HCC cell lines and expression of AFP was measured by quantitative RT-PCR. The vertical axis is AFP gene expression of each cell line relative to that of PLC/PRF/5 cells. The cell lines which are sensitive or insensitive to OSI-906 are indicated.

FIG. 3: AFP expression correlates with sensitivity to OSI-906 in HCC cell lines. The top panel illustrates the sensitivity of 21 HCC cell lines using EC50 (μM) values. The lower panel shows the AFP gene index score of each cell line. Cell lines used for both panels are indicated at the bottom of the lower panel. Index score calculation is as described in the materials and methods section of the Experimental Details section herein.

FIG. 4: AFP gene expression level in HCC cells correlates with AFP protein concentration in the growth media, resulting from secreted AFP. All the HCC cell lines sensitive to OSI-906, and some insensitive HCC cell lines, were grown for 3 days. The AFP concentration in the growth medium was measure by ELISA (R&D Systems Inc., Minneapolis, Minn.), and normalized against total cell number (top panel). The bottom panel shows AFP gene index score. The correlation coefficient and p-value are indicated.

FIG. 5: Serum AFP level and tumor AFP mRNA expression are highly correlated. AFP gene expression level (top panel) and serum concentration (bottom panel) from a published HCC tumor database were grouped according to a clinical classification as described in Chiang, D. Y. et al. (2008) Cancer Res. 68:6779-6788. Pearson correlation was performed. The correlation coefficient and p-value are indicated. HCC tumors expressing high levels of AFP are enriched in the proliferation subgroup.

FIG. 6: IGF axis 4-gene index scores correlate with sensitivity to OSI-906. IGF axis 4-gene index scores were calculated as described herein for each of the HCC cell lines indicated (top panel). A higher index score means higher IGF-2/INSR expression and lower IGFBP3/7 expression. EC50 values from proliferation assays after OSI-906 treatment of the HCC cells (bottom panel) were used to calculate the correlation.

FIG. 7: E-cadherin mRNA expression correlates with sensitivity to OSI-906. E-cadherin expression in 21 HCC cell lines was measured, and median-centered values were used as E-cadherin index score (top panel). The effect of OSI-906 on proliferation in each HCC cell line was tested and EC50 was shown (bottom panel).

FIG. 8: Expression of EMT genes in HCC cells. Expression levels of 12 genes associated with mesenchymal cells and 7 genes associated with epithelial cells were assessed with RT-PCR and analyzed using a heatmap. Red color and green color represent the maximal and minimal expression of each gene within the 21 HCC cell lines, respectively. Cell lines sensitive to OSI-906 are indicated by the red colored font (i.e. JHH-5, HUH-6, Hep3B, JHH-7, HUH-1, HepG2, HUH-7).

FIG. 9: Expression of EMT markers in HCC cells. HCC cells were lysed and expressions of E-cadherin, ErbB3, Vimentin and Zebl were measured by western blotting. Actin was used as loading control. EMT index scores were listed and EMT status was indicated at the bottom.

FIG. 10: AFP expression is restricted to epithelial HCC cells. AFP gene expression in 21 HCC cell lines were measured and median-centered values were used as AFP index scores. Epithelial cells that were assessed based on previous results (from FIG. 1-3) are indicated.

FIG. 11: TGFβ decreases AFP expression and cell sensitivity to OSI-906. HUH-1, JHH-5 and HepG2 cells were treated with TGFβ and cells were either treated with OSI-906 for proliferation assays (A) or lysed to measure AFP expression by RT-PCR (B).

FIG. 12: Combination of OSI-906 and Erlotinib confers synergy in epithelial HCC cells. Two sensitive epithelial cell lines Hep3B (A), JHH-7 (B), one insensitive epithelial cell line JHH-1(C) or one insensitive mesenchymal-like cell line HLF (D) were treated serial dilution of OSI-906 in the presence of Erlotinib for 72 hours, and sensitivity to treatment was measured by proliferation assays. The concentrations of Erlotinib used were determined beforehand to inhibit about 50% proliferation (Hep3B: 3.3 μM; JHH-1: 0.12 μM) or maximal concentration (10 μM) was used (JHH-7, HLF).

FIG. 13: HCC reference cell lines that define expression threshold. RT-PCR was performed with total RNA isolated from HCC cell lines. Delta CT values were used to calculate the relative expression levels of IGF-2, IR, IGFBP7, and IGFBP3 (see Material & Methods herein).

### DETAILED DESCRIPTION OF THE INVENTION

The term “cancer” in a patient refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within the subject, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means to give medical aid to counteract a disease or condition. The phrase “a method of treating” or its equivalent, when applied to cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed an overall beneficial course of action.

The term “therapeutically effective agent” means a composition that will elicit the biological or medical response of a tissue, system, or human that is being sought by the researcher, medical doctor or other clinician.

The term “therapeutically effective amount” or “effective amount” means the amount of the subject compound or combination that will elicit the biological or medical response of a tissue, system, or human that is being sought by the researcher, medical doctor or other clinician.

The terms “responsive” or “responsiveness” when used herein in referring to a patient's reaction to administration of an IGF-1R kinase inhibitor, refers to a response that is positive or effective, from which the patient is likely to benefit.

The NCBI GeneID numbers listed herein are unique identifiers of genes from the NCBI Entrez Gene database record (National Center for Biotechnology Information (NCBI), U.S. National Library of Medicine, 8600 Rockville Pike, Building 38A, Bethesda, Md. 20894; Internet address http://www.ncbi.nlm.nih.gov/).

The data presented in the Experimental Details section herein below demonstrates that HCC tumor cells show a range of sensitivities to growth inhibition by an IGF-1R kinase inhibitor (e.g. OSI-906) and that the degree of sensitivity of the tumor cells to an IGF-1R kinase inhibitor can be assessed by determining the degree of expression of AFP in the tumor cells, such that high expression is indicative that the cells are likely to have high sensitivity to growth inhibition by an IGF-1R kinase inhibitor, or conversely, low expresion is indicative that the cells are likely to have have low sensitivity, or be relatively resistant, to growth inhibition by an IGF-1R kinase inhibitor. Thus, these observations can form the basis of valuable new diagnostic methods for predicting the effects of IGF-1R kinase inhibitors on HCC tumor growth, and give oncologists an additional biomarker to assist them in choosing the most appropriate treatment for their patients.

Since AFP is a protein that is secreted from HCC cells, and it has been shown herein that in humans there is a high degree of correlation between the level of AFP mRNA expressed in HCC cells and the level of AFP protein present in serum, the level of AFP in serum can also be used as a predictor of HCC sensitivity to IGF-1R kinase inhibitors, such that high serum levels are indicative that the HCC cells are likely to have high sensitivity to growth inhibition by an IGF-1R kinase inhibitor, or conversely, low serum levels are indicative that the HCC cells are likely to have have low sensitivity, or be relatively resistant, to growth inhibition by an IGF-1R kinase inhibitor.

The data presented in the Experimental Details section herein below also demonstrates that the EMT status of HCC tumor cells may determine their sensitivity to growth inhibition by an IGF-1R kinase inhibitor. Thus, the degree of sensitivity of the HCC tumor cells to an IGF-1R kinase inhibitor can be assessed by determining the degree of expression of an epithelial biomarker in the tumor cells, such that high expression is indicative that the cells are likely to have high sensitivity to growth inhibition by an IGF-1R kinase inhibitor, or conversely, low expresion is indicative that the cells are likely to have have low sensitivity, or be relatively resistant, to growth inhibition by an IGF-1R kinase inhibitor. Similarly, the degree of sensitivity of the HCC tumor cells to an IGF-1R kinase inhibitor can be assessed by determining the degree of expression of a mesenchymal biomarker in the tumor cells, such that low expression is indicative that the cells are likely to have high sensitivity to growth inhibition by an IGF-1R kinase inhibitor, or conversely, high expresion is indicative that the cells are likely to have have low sensitivity, or be relatively resistant, to growth inhibition by an IGF-1R kinase inhibitor. Thus, these observations can also form the basis of valuable new diagnostic methods for predicting the effects of IGF-1R kinase inhibitors on HCC tumor growth, and give oncologists additional biomarkers to assist them in choosing the most appropriate treatment for their patients.

The data presented in the Experimental Details section herein below also demonstrates that the degree of sensitivity of HCC tumor cell growth to an IGF-1R kinase inhibitor can be assessed by determining the degree of expression of INSR, IGF-2, IGFBP3 or IGFBP7 in the HCC tumor cells. High expression of IR or IGF-2 is indicative that the cells are likely to have high sensitivity to growth inhibition by an IGF-1R kinase inhibitor, or conversely, low expresion of INSR or IGF-2 is indicative that the cells are likely to have have low sensitivity, or be relatively resistant, to growth inhibition by an IGF-1R kinase inhibitor. High expression of IGFBP3 or IGFBP7 is indicative that the cells are likely to have low sensitivity to growth inhibition by an IGF-1R kinase inhibitor, or conversely, low expresion of IGFBP3 or IGFBP7 is indicative that the cells are likely to have have high sensitivity, or be relatively resistant, to growth inhibition by an IGF-1R kinase inhibitor. A 4-gene signature (i.e. 4GS) index score (i.e. 4-gene index score) calculated using the HCC expression values for each of these four genes (see below for equation) was also found to significantly correlate with sensitivity of HCC tumor cells to an IGF-1R kinase inhibitor, and to a much greater degree than any individual gene expression values. High 4GS index scores (i.e. scores of a higher magnitude, the sign of which will depend on the method of gene expression analysis utilized, e.g. high negative scores for RTPCR; high positive score for gene array analysis) are found to be indicative of high HCC tumor cell sensitivity to an IGF-1R kinase inhibitor (i.e. low EC50 values). These observations provide the basis for additional diagnostic methods for predicting the effects of IGF-1R kinase inhibitors on HCC tumor growth, giving oncologists additional biomarkers to assist them in choosing the most appropriate treatment for their patients.

IGF Axis 4-Gene Index Equation:

$$IGF axis 4 \text{-} gene index score = \frac{1}{n} {\sum }_{i ∈ IGF} {g}_{i} \cdot r$$

wherein IGF=genes in the IGF axis: IGF2, INSR, IGFBP3, and IGFBP7,

n=number of genes in the IGF axis=4, and

gi=median centered expression value of gene i.

r=+1 for IGF2 and INSR; r=−1 for IGFBP3 and IGFBP7.

AFP, INSR, IGF-2, IGFBP3 and IGFBP7 as used herein refer respectively to the following human genes: alpha-fetoprotein (NCBI GeneID number 174), insulin receptor (NCBI GeneID number 3643); insulin-like growth factor 2 (NCBI GeneID number 3481; also known as somatomedin A); insulin-like growth factor binding protein 3 (NCBI GeneID number 3486); and insulin-like growth factor binding protein 7 (NCBI GeneID number 3490).

ACTN1, SPARC, ITGB3, PLAUR, CDH2, SNAIL SNAI2, TWIST1, VCAN, VIM, ZEB1, ZEB2, CDH1, CLDN3, ERBB3, MTA3, MAP7, TJP3, and OCLN as used herein refer respectively to the following human genes: ACTN1 (NCBI GeneID number 87), SPARC(NCBI GeneID number 6678), ITGB3 (NCBI GeneID number 3690), PLAUR(NCBI GeneID number 5329), CDH2 (NCBI GeneID number 1000), SNAI1 (NCBI GeneID number 6615), SNAI2 (NCBI GeneID number 6591), TWIST1 (NCBI GeneID number 7291), VCAN(NCBI GeneID number 1462), VIM (NCBI GeneID number 7431), ZEB1 (NCBI GeneID number 6935), ZEB2 (NCBI GeneID number 9839), CDH1 (NCBI GeneID number 999), CLDN3 (NCBI GeneID number 1365), ERBB3 (NCBI GeneID number 2065), MTA3 (NCBI GeneID number 57504), MAP7 (NCBI GeneID number 9053), TJP3 (NCBI GeneID number 27134), and OCLN (NCBI GeneID number 4950).

In any of the methods described herein where gene expression of INSR, IGF2 or IGFBP3 is assessed, levels of all transcripts or their protein products are assessed (i.e. including transcripts coding for different protein isoforms). As an alternative to any of these methods where levels of all transcripts are assessed, the level of only one transcript type, or a number less than the total number of different types of transcript, may be determined.

In the context of this invention, the sensitivity of tumor cell growth to the IGF-1R kinase inhibitor OSI-906 is defined as high if the tumor cell is inhibited with an EC50 (half-maximal effective concentration) of less than 1 μM and at least 40% inhibition at 5 μM OSI-906, and low (i.e. relatively resistant) if the tumor cell is inhibited with an EC50 of greater than 10 μM. Sensitivies between these values are considered intermediate. With other IGF-1R kinase inhibitors, particularly compounds of Formula I as described herein below, a qualitatively similar result is expected since they inhibit tumor cell growth by inhibiting the same signal transduction pathway, although quantitatively the EC50 values may differ depending on the relative cellular potency of the other inhibitor versus OSI-906. Thus, for example, the sensitivity of tumor cell growth to a more potent IGF-1R kinase inhibitor would be defined as high when the tumor cell is inhibited with an EC50 that is correspondingly lower. In tumor xenograft studies, using tumor cells of a variety of tumor cell types that all have high sensitivity to OSI-906 in culture in vitro, the tumors are consistently inhibited in vivo with a high pencentage tumor growth inhibition (TGI) (see U.S. provisional application No. 61/310,031). In contast, in similar studies, using tumor cells that have low sensitivity to OSI-906 in culture in vitro, the tumors are inhibited in vivo with only a low pencentage tumor growth inhibition (TGI). These data indicate that sensitivity to IGF-1R kinase inhibitors such as OSI-906 in tumor cell culture is predictive of tumor sensitivity in vivo.

The term EC50 (half maximal effective concentration) refers to the concentration of compound which induces a response halfway between the baseline and maximum for the specified exposure time, and is used as a measure of the compound's potency. Lower EC50 values are found when a tumor cell is more sensitive to the effects of a compound.

The present invention thus provides a method of predicting the sensitivity of hepatocellular carcinoma cell growth to an IGF-1R kinase inhibitor, comprising: determining whether the HCC tumor cells express a high level of AFP; and predicting that tumor cell growth is likely to be sensitive to an IGF-1R kinase inhibitor if the tumor cells express a high level of AFP. This method may be utilized to select a cancer patient who is predicted to benefit from therapeutic administration of an IGF-1R kinase inhibitor, by applying it to a sample of the cells of a tumor of the patient (e.g. a tumor biopsy, or circulating tumor cells isolated from a blood sample), either alone, or in addition to other diagnostic tests to predict response to administration of an IGF-1R kinase inhibitor. The present invention thus provides a method of identifying patients with hepatocellular carcinoma who are most likely to benefit from treatment with an IGF-1R kinase inhibitor, comprising: obtaining a sample of a patient's tumor; determining whether the tumor cells express a high level of AFP; and identifying the patient as one most likely to benefit from treatment with an IGF-1R kinase inhibitor if the tumor cells express a high level of AFP. Inherent in this method is the recognition that the presence of a high expression level of AFP in hepatocellular carcinoma cells correlates with higher sensitivity of the HCC tumor cells to growth inhibition by an IGF-1R kinase inhibitor than HCC tumor cells that have a low expression level of AFP. In an alternative embodiment of these methods, instead of assessing AFP expression directly in an HCC tumor sample, AFP expression may be assessed by determining the level of AFP secreted by the HCC tumor cells into a patients serum. A determination of whether AFP expression is high or low can readily be assessed by measuring a patient's AFP level relative to a control or reference level, as described further herein.

The present invention also provides a method of predicting the sensitivity of hepatocellular carcinoma cell growth to inhibition by an IGF-1R kinase inhibitor, comprising: determining if the hepatocellular carcinoma cells express a high level of AFP; and concluding that if the tumor cells express a high level of AFP, high sensitivity to growth inhibition by IGF-1R kinase inhibitors is predicted, based upon a predetermined correlation of the presence of high AFP expression with high sensitivity.

The present invention also provides method for treating hepatocellular carcinoma in a patient, comprising the steps of: predicting the sensitivity of hepatocellular carcinoma cell growth to inhibition by an IGF-1R kinase inhibitor, by determining if the hepatocellular carcinoma cells express a high level of AFP; and concluding that if the HCC tumor cells express a high level of AFP, high sensitivity to growth inhibition by IGF-1R kinase inhibitors is predicted, based upon a predetermined correlation of the presence of high AFP expression with high sensitivity; and administering to said patient a therapeutically effective amount of an IGF-1R kinase inhibitor if high sensitivity of the hepatocellular carcinoma cells to growth inhibition by IGF-1R kinase inhibitors is predicted.

The present invention also provides a method of identifying patients with hepatocellular carcinoma who are most likely to benefit from treatment with an IGF-1R kinase inhibitor, comprising: determining whether the hepatocellular carcinoma cells express a high level of AFP; and identifying the patient as one most likely to benefit from treatment with an IGF-1R kinase inhibitor if the hepatocellular carcinoma cells express a high level of AFP.

The present invention also provides a method of identifying patients with hepatocellular carcinoma who are most likely to benefit from treatment with an IGF-1R kinase inhibitor, comprising: obtaining a sample of a patient's tumor, determining if tumor cells of the sample express a high level of AFP; and identifying the patient as likely to benefit from treatment with an IGF-1R kinase inhibitor if high AFP expression is present in the tumor cells of the patient.

The present invention also provides a method for treating hepatocellular carcinomas (HCC) or HCC tumor metastases in a patient, comprising the steps of: assessing a patient's likely responsiveness to an IGF-1R kinase inhibitor by determining if the hepatocellular carcinoma cells of the patient express a high level of AFP, identifying the patient as likely to benefit from treatment with an IGF-1R kinase inhibitor if high AFP expression is present in the hepatocellular carcinoma cells of the patient, and administering to said patient a therapeutically effective amount of an IGF-1R kinase inhibitor.

The invention further provides a method for treating hepatocellular carcinoma in a patient, comprising the steps of: (A) assessing a patient's likely responsiveness to an IGF-1R kinase inhibitor by determining if the patient has an HCC tumor that is likely to respond to treatment with an IGF-1R kinase inhibitor by: obtaining a sample of the patient's tumor; determining whether the tumor cells express a high level of AFP; and identifying the patient as likely to benefit from treatment with an IGF-1R kinase inhibitor if the tumor cells express a high level of AFP, and (B) administering to said patient a therapeutically effective amount of an IGF-1R kinase inhibitor if the patient is diagnosed to be potentially responsive to an IGF-1R kinase inhibitor.

The invention further provides a method of identifying patients with hepatocellular carcinoma who are most likely to benefit from treatment with an IGF-1R kinase inhibitor in combination with an other anti-cancer agent, comprising: obtaining a sample of a patient's HCC tumor, determining if the HCC tumor cells of the sample express a high level of AFP; and identifying the patient as likely to benefit from treatment with an IGF-1R kinase inhibitor in combination with an other anti-cancer agent if the tumor cells express a high level of AFP. In one embodiment of this method the other anti-cancer agent comprises a small molecule EGFR kinase inhibitor, e.g. erlotinib, gefitinib, or lapatinib. In another embodiment of this method the other anti-cancer agent comprises an antibody EGFR kinase inhibitor, e.g. cetuximab, zalutumumab, nimotuzumab, or matuzumab.

The invention further provides a method for treating hepatocellular carcinoma in a patient, comprising the steps of: (A) assessing a patient's likely responsiveness to an IGF-1R kinase inhibitor in combination with an other anti-cancer agent, by determining if the patient has an HCC tumor that is likely to respond to treatment with an IGF-1R kinase inhibitor in combination with an other anti-cancer agent by: obtaining a sample of the patient's HCC tumor; determining whether the tumor cells express a high level of AFP; and identifying the patient as likely to benefit from treatment with an IGF-1R kinase inhibitor in combination with an other anti-cancer agent if the tumor cells express a high level of AFP, and (B) administering to said patient a therapeutically effective amount of an IGF-1R kinase inhibitor in combination with an other anti-cancer agent if the patient is diagnosed to be potentially responsive to an IGF-1R kinase inhibitor in combination with an other anti-cancer agent. In one embodiment of this method the other anti-cancer agent comprises a small molecule EGFR kinase inhibitor, e.g. erlotinib, gefitinib, or lapatinib. In another embodiment of this method the other anti-cancer agent comprises an antibody EGFR kinase inhibitor, e.g. cetuximab, zalutumumab, nimotuzumab, or matuzumab.

The other anti-cancer agent of any of the methods of this invention which comprise a step of “identifying patients with hepatocellular carcinoma who are most likely to benefit from treatment with an IGF-1R kinase inhibitor in combination with another anti-cancer agent” may for example be selected from the following agents: erlotinib, cetuximab, gefitinib, lapatinib, panitumumab, zalutumumab, nimotuzumab, and matuzumab. In these methods the combination of these agents will generally act in a synergistic fashion to inhibit HCC.

The present invention further provides a method for treating hepatocellular carcinomas (HCC) or HCC tumor metastases in a patient, comprising the steps of assessing a patient's likely responsiveness to an IGF-1R kinase inhibitor using any of the methods described herein for determining that the HCC tumor cells have a high expression level of AFP, or that the HCC patient has a high level of serum AFP, and administering to said patient a therapeutically effective amount of an IGF-1R kinase inhibitor. For this method, an example of a preferred IGF-1R kinase inhibitor is OSI-906, or a compound with similar characteristics (e.g. selectivity, potency), including pharmacologically acceptable salts or polymorphs thereof. In this method one or more additional anti-cancer agents or treatments can be co-administered simultaneously or sequentially with the IGF-1R kinase inhibitor, as judged to be appropriate by the administering physician given the prediction of the likely responsiveness of the patient to an IGF-1R kinase inhibitor, combined with any additional circumstances pertaining to the individual patient.

It will be appreciated by one of skill in the medical arts that the exact manner of administering to a patient with a hepatocellular carcinoma, a therapeutically effective amount of an IGF-1R kinase inhibitor following a diagnosis of a patient's likely responsiveness to an IGF-1R kinase inhibitor, will be at the discretion of the attending physician. The mode of administration, including dosage, combination with other anti-cancer agents, timing and frequency of administration, and the like, may be affected by the diagnosis of a patient's likely responsiveness to an IGF-1R kinase inhibitor, as well as the patient's condition and history. Thus, even patients diagnosed with hepatocellular carcinomas predicted to be relatively insensitive to IGF-1R kinase inhibitors may still benefit from treatment with such inhibitors, particularly in combination with other anti-cancer agents, or agents that may alter a tumor's sensitivity to IGF-1R kinase inhibitors.

The present invention further provides a method for treating hepatocellular carcinomas (HCC) or HCC tumor metastases in a patient, comprising the steps of assessing a patient's likely responsiveness to an IGF-1R kinase inhibitor by assessing whether the tumor cells are sensitive to inhibition by an IGF-1R kinase inhibitor, by for example any of the methods described herein for determining the presence of high AFP expression in HCC tumor cells, identifying the patient as one who is likely to demonstrate an effective response to treatment with an IGF-1R kinase inhibitor, and administering to said patient a therapeutically effective amount of an IGF-1R kinase inhibitor. In one embodiment the IGF-1R kinase inhibitor used for treatment comprises OSI-906.

The present invention also provides a method for inhibiting hepatocellular carcinoma cell growth in a patient, comprising the steps of assessing a patient's likely responsiveness to an IGF-1R kinase inhibitor by using any of the methods described herein to predict the sensitivity of HCC tumor cell growth to inhibition by an IGF-1R kinase inhibitor, identifying the patient as one who is likely to demonstrate an effective response to treatment with an IGF-1R kinase inhibitor, and administering to said patient a therapeutically effective amount of an IGF-1R kinase inhibitor. In one embodiment the IGF-1R kinase inhibitor used for treatment comprises OSI-906.

In one embodiment of any of the methods described herein that involve assessment of AFP gene expression, the term “high AFP expression” (or “high level of AFP”) means the AFP expression level, or greater, that is found in a reference HCC tumor cell that is known to have high AFP expression, such as Hep3B or HUH1 cells. In a sample of HCC tumor cells from a patient, it may be determined whether the AFP level is high by making a comparison to such a reference HCC tumor cell that is known to have high AFP. In an alternative embodiment of any of the methods described herein that involve assessment of AFP gene expression, the term “high AFP expression” (or “high level of AFP”) means the AFP expression level, or greater, that is found in a reference HCC tumor cell that is known to have high AFP expression, wherein the AFP expression level is higher than that in the reference cells Hep3B or HUH1, e.g. as in HUH-6, HUH-7, JHH-5, JHH-7, or HepG2 cells. Additional reference cells with AFP expression levels similar to any of the reference cells mentioned herein may be used as reference cells in the methods of the invention described herein.

For any of the methods of this invention in which a reference cell level is used in the determination of the level of a biomarker (e.g. AFP, E-cadherin, 4-GS index etc.), wherein the reference cell is chosen to indicate a threshold level between high and low levels of biomarker, one of skill in the art would typically choose a reference cell with high expression of the biomarker, at a level that is predictive of sensitivity or responsiveness to an IGF-1R kinase inhibitor, but at the lower end of the range of expression of the biomarker, such that all or most tumor samples to which it will be compared will have a higher level of biomarker expression if they are sensitive to an IGF-1R kinase inhibitor. Alternatively, a reference cell with higher biomarker expression than such a reference cell may be chosen in order to minimize any issue of false positives that may arise.

The present invention further provides a method for treating hepatocellular carcinomas (HCC) or HCC tumor metastases in a patient, comprising the steps of assessing a patient's likely responsiveness to an IGF-1R kinase inhibitor by any of the methods described herein for determining low expression levels of AFP, identifying the patient as one who is less likely or not likely to demonstrate an effective response to treatment with an IGF-1R kinase inhibitor, and treating said patient with an anti-cancer therapy other than an IGF-1R kinase inhibitor. In one embodiment of this method, the anti-cancer therapy other than an IGF-1R kinase inhibitor is a standard treatment for hepatocellular carcinoma, e.g. hepatectomy, liver transplantation, radiation therapy, sorafenib.

The present invention also provides a method of identifying patients with hepatocellular carcinoma (HCC) who are most likely to benefit from treatment with an IGF-1R kinase inhibitor, comprising: obtaining a serum sample from the patient; determining the level of AFP protein in the serum sample; and identifying the patient as likely to benefit from treatment with an IGF-1R kinase inhibitor if the serum contains a high level of AFP. The present invention also provides a method of identifying patients with hepatocellular carcinoma (HCC) who are most likely to benefit from treatment with an IGF-1R kinase inhibitor, comprising: obtaining a blood sample from the patient; determining the level of AFP protein in the blood sample; and identifying the patient as likely to benefit from treatment with an IGF-1R kinase inhibitor if the blood contains a high level of AFP. The present invention also provides a method of identifying patients with hepatocellular carcinoma (HCC) who are most likely to benefit from treatment with an IGF-1R kinase inhibitor, comprising: obtaining a plasma sample from the patient; determining the level of AFP protein in the plasma sample; and identifying the patient as likely to benefit from treatment with an IGF-1R kinase inhibitor if the plasma contains a high level of AFP.

The present invention also provides a method of identifying patients with hepatocellular carcinoma (HCC) who are most likely to benefit from treatment with an IGF-1R kinase inhibitor, comprising: determining the level of AFP protein in the serum (or blood, or plasma) of a patient; and identifying the patient as likely to benefit from treatment with an IGF-1R kinase inhibitor if the serum (or blood, or plasma) contains a high level of AFP.

The present invention further provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a serum (or blood, or plasma) sample from the patient; determining the level of AFP protein in the serum (or blood, or plasma) sample; identifying the patient as likely to benefit from treatment with an IGF-1R kinase inhibitor if the serum (or blood, or plasma) contains a high level of AFP, and (B) administering to said patient a therapeutically effective amount of an IGF-1R kinase inhibitor if the patient is diagnosed to be potentially responsive to an IGF-1R kinase inhibitor.

The present invention further provides a method for treating hepatocellular carcinoma in a patient, comprising administering to said patient a therapeutically effective amount of an IGF-1R kinase inhibitor if the patient has been diagnosed to be potentially responsive to an IGF-1R kinase inhibitor by a determination that the serum (or blood, or plasma) of the patient contains a high level of AFP.

In any of the methods described herein where serum (or blood, or plasma) AFP is level is, or has been, determined, total AFP levels are assessed, i.e. the method assays all AFP isoforms (i.e. L1, L2, and L3). As an alternative method to any of these methods where total AFP is determined, the level of only the L3 isoform may be determined.

In different embodiments of any of the methods of this invention, a “high” level of serum (or blood, or plasma) AFP may be equal to or greater than a value of 20, 30, 40, 50, 60, 70, 80, 90, 100, 150, 200, 250, 300, 350, 400, 450, 500, 550, 60, 650, 700, 750, 800, 900, or 1000 ng AFP/ml serum (or blood, or plasma). In alternative embodiments of any of the methods of this invention a “high” level of serum (or blood, or plasma) AFP may be equal to or greater than any integer ng AFP/ml value between any consecutive two of those listed values. It is desirable to be able to choose different thresholds for the level of “high” AFP in assays in order optimize the assay in different circumstances (e.g. different patient populations), where the level of false positives or false negatives may vary depending on the particular threshold chosen.

The present invention also provides a method of identifying patients with hepatocellular carcinoma (HCC) who are most likely to benefit from treatment with an IGF-1R kinase inhibitor, comprising: obtaining a sample of a patient's HCC tumor; determining whether the HCC tumor cells express a high level of an epithelial biomarker; and identifying the patient as one most likely to benefit from treatment with an IGF-1R kinase inhibitor if the tumor cells express a high level of an epithelial biomarker. A determination of whether an epithelial biomarker expression is high or low can readily be assessed by measuring a patient's epithelial biomarker level relative to a control or reference level, as described further herein.

The present invention further provides a method of identifying patients with hepatocellular carcinoma (HCC) who are most likely to benefit from treatment with an IGF-1R kinase inhibitor, comprising: obtaining a sample of a patient's HCC tumor; determining whether the HCC tumor cells express a low level of a mesenchymal biomarker; and identifying the patient as one most likely to benefit from treatment with an IGF-1R kinase inhibitor if the tumor cells express a low level of a mesenchymal biomarker. A determination of whether a mesenchymal biomarker expression is high or low can readily be assessed by measuring a patient's mesenchymal biomarker level relative to a control or reference level, as described further herein.

The present invention further provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a sample of the patient's tumor; determining whether the tumor cells express a high level of an epithelial biomarker and/or a low level of a mesenchymal biomarker; and identifying the patient as likely to benefit from treatment with an IGF-1R kinase inhibitor if the tumor cells express a high level of an epithelial biomarker and/or a low level of a mesenchymal biomarker, and (B) administering to said patient a therapeutically effective amount of an IGF-1R kinase inhibitor if the patient is diagnosed to be potentially responsive to an IGF-1R kinase inhibitor.

The present invention further provides a method of predicting the sensitivity of hepatocellular carcinoma cell growth to inhibition by an IGF-1R kinase inhibitor, comprising: determining if the hepatocellular carcinoma cells express a high level of an epithelial biomarker and/or a low level of a mesenchymal biomarker; and concluding that if the hepatocellular carcinoma cells express a high level of an epithelial biomarker and/or a low level of a mesenchymal biomarker, high sensitivity to growth inhibition by IGF-1R kinase inhibitors is predicted, based upon a predetermined correlation of the presence of a high level of an epithelial biomarker and/or a low level of a mesenchymal biomarker with said high sensitivity.

The present invention further provides a method of identifying patients with hepatocellular carcinoma who are most likely to benefit from treatment with an IGF-1R kinase inhibitor in combination with an EGFR kinase inhibitor, comprising: obtaining a sample of a patient's tumor; determining whether the tumor cells express a high level of an epithelial biomarker and/or a low level of a mesenchymal biomarker; and identifying the patient as one most likely to benefit from treatment with an IGF-1R kinase inhibitor in combination with an EGFR kinase inhibitor if the tumor cells express a high level of an epithelial biomarker and/or a low level of a mesenchymal biomarker. In one embodiment of this method the EGFR kinase inhibitor comprises a small molecule EGFR kinase inhibitor, e.g. erlotinib, gefitinib, or lapatinib. In another embodiment of this method the EGFR kinase inhibitor comprises an antibody EGFR kinase inhibitor, e.g. cetuximab, zalutumumab, nimotuzumab, or matuzumab.

The present invention further provides a method for treating hepatocellular carcinoma in a patient, comprising the steps of: (A) assessing a patient's likely responsiveness to an IGF-1R kinase inhibitor in combination with an EGFR kinase inhibitor by determining if the patient has an hepatocellular carcinoma that is likely to respond to treatment with such a combination by: obtaining a sample of the patient's tumor; determining whether the tumor cells express a high level of an epithelial biomarker and/or a low level of a mesenchymal biomarker; and identifying the patient as likely to benefit from treatment with an IGF-1R kinase inhibitor in combination with an EGFR kinase inhibitor if the tumor cells express a high level of an epithelial biomarker and/or a low level of a mesenchymal biomarker, and (B) administering to said patient a therapeutically effective amount of an IGF-1R kinase inhibitor in combination with an EGFR kinase inhibitor if the patient is diagnosed to be potentially responsive to such a combination. In one embodiment of this method the EGFR kinase inhibitor comprises a small molecule EGFR kinase inhibitor, e.g. erlotinib, gefitinib, or lapatinib. In another embodiment of this method the EGFR kinase inhibitor comprises an antibody EGFR kinase inhibitor, e.g. cetuximab, zalutumumab, nimotuzumab, or matuzumab.

The present invention further provides a method for treating hepatocellular carcinoma in a patient, comprising administering to said patient a therapeutically effective amount of an IGF-1R kinase inhibitor if the patient has been diagnosed to be potentially responsive to an IGF-1R kinase inhibitor by a determination that the tumor cells of the patient express a high level of an epithelial biomarker and/or a low level of a mesenchymal biomarker.

In any of the methods described herein wherein the expression of an epithelial biomarker is determined, the epithelial biomarker may be the gene CDH1, CLDN3, ERBB3, MTA3, MAP7, TJP3, or OCLN, wherein mRNA or protein expression products thereof may be determined.

In any of the methods described herein wherein the expression of a mesenchymal biomarker is determined, the mesenchymal biomarker may be the gene ACTN1, SPARC, ITGB3, PLAUR, CDH2, SNAIL SNAI2, TWIST1, VCAN, VIM, ZEB1, or ZEB2, wherein mRNA or protein expression products thereof may be determined.

In any of the methods described herein where epithelial gene expression is assessed, a high level of an epithelial biomarker may be a level equal to or greater than a level found in a reference HCC tumor cell that has high sensitivity to growth inhibition by an IGF-1R kinase inhibitor. For example, the reference HCC tumor cell may be HUH1 or HUH7. Alternatively, a cell of another type with similar levels of an epithelial biomarker may be used as a reference.

Similarly, in any of the methods described herein where mesenchymal gene expression is assessed, a low level of a mesenchymal biomarker is a level less than the level found in a reference HCC tumor cell that has low sensitivity to growth inhibition by an IGF-1R kinase inhibitor. For example, the reference HCC tumor cell may be SNU-475 or SNU-398. Alternatively, a cell of another type with similar levels of an epithelial biomarker may be used as a reference.

The present invention further provides any of the methods described herein, wherein the sample of a patient's tumor is derived from a tumor biopsy, or wherein the sample of a patient's tumor is derived from a blood sample containing circulating tumor cells.

The present invention further provides any of the methods described herein, wherein the IGF-1R kinase inhibitor comprises a small molecule IGF-1R kinase inhibitor, e.g. OSI-906. The present invention further provides any of the methods described herein, wherein the IGF-1R kinase inhibitor comprises is an anti-IGF-1R antibody or antibody fragment. The present invention further provides any of the methods described herein, wherein one or more additional anti-cancer agents are co-administered simultaneously or sequentially with the IGF-1R kinase inhibitor that is administered.

The present invention provides a method of identifying patients with hepatocellular carcinoma (HCC) who are most likely to benefit from treatment with an IGF-1R kinase inhibitor, comprising:

measuring in a sample of HCC tumor cells from the patient the relative expression level of each gene of a 4-gene signature (4GS), wherein the 4GS consists of the following genes: INSR, IGF2, IGFBP3 and IGFBP7; calculating a 4GS index score for said tumor cells according to the equation:

$$4 GS index score = \frac{1}{n} {\sum }_{i ∈ IGF} {g}_{i} \cdot r ,$$

wherein: IGF=the genes: IGF2, INSR, IGFBP3, and IGFBP7; n=number of genes in the gene signature=4; and gᵢ=median centered expression value of gene i; and r=+1 for IGF2 and INSR, and r=−1 for IGFBP3 and IGFBP7; determining if said 4GS index score is more similar to a 4GS index score from a reference HCC tumor cell that is sensitive to growth inhibition by an IGF-1R kinase inhibitor or an 4GS index score from a reference HCC tumor cell that is resistant to growth inhibition by an IGF-1R kinase inhibitor, and identifying the patient as one likely to benefit from treatment with an IGF-1R kinase inhibitor if their HCC tumor cells have a 4GS index score that is more similar to a 4GS index score from a reference HCC tumor cell that is sensitive to growth inhibition by an IGF-1R kinase inhibitor. In one embodiment of this method, the additional step of obtaining a sample of cells of the tumor of the patient prior to the step of measuring expression levels is included. The sample of tumor cells may for example be derived from a tumor biopsy or from a blood sample containing circulating tumor cells. The present invention further provides a method for treating a patient with a hepatocellular carcinoma, comprising the steps of: diagnosing a patient's likely responsiveness to an IGF-1R kinase inhibitor using this method, and administering to said patient a therapeutically effective amount of an IGF-1R kinase inhibitor if the patient is predicted to benefit from treatment with an IGF-1R kinase inhibitor. The present invention thus provides a method of treatment of a patient with hepatocellular carcinoma, comprising: administering to said patient a therapeutically effective amount of an IGF-1R kinase inhibitor if the patient is predicted to be responsive to an IGF-1R kinase inhibitor using this method.

Assessment of an index score of a patient's HCC tumor cells as more similar to an index score of a reference HCC tumor cell that is sensitive to growth inhibition by an IGF-1R kinase inhibitor, or a reference HCC tumor cell that is resistant to growth inhibition by an IGF-1R kinase inhibitor, in any of the methods of this invention, may be determined by comparison to the value of the index score of a reference or control HCC tumor cell sample, wherein this HCC reference tumor cell score has been previously correlated with either IGF-1R kinase inhibitor sensitivity or resistance. Alternatively, a panel of such reference HCC tumor cell samples, representing a range of index scores, and thus a range of IGF-1R kinase inhibitor sensitivities, can be used construct a standard curve from which the sensitivity can be predicted from the index score for test tumor cell samples.

The term “more similar” as used herein has its usual meaning. HCC tumor cells from patients will have a range of index scores reflecting the IGF-1R kinase inhibitor sensitivities of the cells, for example from tumor cells that are extremely sensitive to tumor cells that are relatively resistant. HCC tumor cells from the extremes of such a range may be utilized as reference tumor cells for comparison to a sample of tumor cells that requires characterization with respect to IGF-1R kinase inhibitor sensitivity. Thus, a tumor cell will be more similar to one or the other of these two extremes if its index score is much closer to the value for the extremely sensitive cell or to the relatively resistant cell (e.g. a reference cell index score plus or minus any value less than 10%, 20%, 30%, 40%, or 50% of the magnitude of the difference between the two reference cell index scores), and would be considered of intermediate sensitivity if its index score falls exactly in the middle of the range. Examples of extremely sensitive or relatively resistant HCC tumor cells that may be used as reference cells in the methods of this invention include HCC tumor samples that have been characterized as extremely sensitive or relatively resistant, or HCC tumor cell lines that have been similarly characterized, including many of those described herein. For example, a JHH-7 or HUH7 cell may be used as a sensitive reference HCC cell, and a SNU 423 or SNU 449 cell may be used as a relatively resistant reference HCC cell. If the index score of sample HCC tumor cells falls outside the range between sensitive and resistant reference cells, it will clearly be more similar to the reference cell on the side of the range where the sample score has fallen outside the range.

It will be appreciated by those of skill in the art that in performing the methods of this invention a reference HCC tumor cell sample(s) need not be established for every assay while the assay is being performed, but rather, a baseline or reference can be established by referring to a form of stored information regarding a previously determined index score(s), or biomarker expression level(s), to discriminate between sensitive and resistant tumor cells (or patient responders and non-responders). Such a form of stored information can include, for example, but is not limited to, a reference chart, listing or electronic file of population or individual data regarding sensitive and resistant tumors or patients, or any other source of data regarding a cutoff level of index value for tumor cell sensitivity or resistance that is useful for the patient or tumor cell to be evaluated.

The present invention also provides a method of identifying a hepatocellular carcinoma (HCC) tumor as likely to be responsive or non-responsive to treatment with an IGF-1R kinase inhibitor, comprising: measuring in a sample of the HCC tumor cells the relative expression level of each gene of a 4-gene signature (4GS), wherein the 4GS consists of the following genes: INSR, IGF2, IGFBP3 and IGFBP7; calculating a 4GS index score for said tumor cells according to the equation:

$$4 GS index score = \frac{1}{n} {\sum }_{i ∈ IGF} {g}_{i} \cdot r ,$$

wherein: IGF=the genes: IGF2, INSR, IGFBP3, and IGFBP7; n=number of genes in the gene signature=4; and gᵢ=median centered expression value of gene i; and r=+1 for IGF2 and INSR; and r=−1 for IGFBP3 and IGFBP7; and determining if the 4GS index score is above a defined threshold that indicates that the tumor is likely to be responsive to an IGF-1R kinase inhibitor (i.e. a high 4GS index score), or below said threshold and thus likely to be non-responsive to an IGF-1R kinase inhibitor (i.e. a low 4GS index score). In one embodiment of this method, the additional step of obtaining a sample of cells of the tumor of the patient prior to the step of measuring expression levels is included. The sample of tumor cells may for example be derived from a tumor biopsy or from a blood sample containing circulating tumor cells. The present invention further provides a method for treating a patient with a hepatocellular carcinoma, comprising the steps of: diagnosing a patient's likely responsiveness to an IGF-1R kinase inhibitor using this method, and administering to said patient a therapeutically effective amount of an IGF-1R kinase inhibitor if the patient is predicted to benefit from treatment with an IGF-1R kinase inhibitor. The present invention thus provides a method of treatment of a patient with hepatocellular carcinoma, comprising: administering to said patient a therapeutically effective amount of an IGF-1R kinase inhibitor if the patient is predicted to be responsive to an IGF-1R kinase inhibitor using this method.

In one embodiment of any of the methods described herein that involve assessment of a 4GS index score, the term “high 4GS index score” means the 4GS index score, or greater, that is found in a reference HCC tumor cell that is known to have a high 4GS index score, such as Hep3B or HepG2 cells. In a sample of HCC tumor cells from a patient, it may be determined whether the 4GS index score is high by making a comparison to such a reference HCC tumor cell that is known to have high 4GS index score. In an alternative embodiment of any of the methods described herein that involve assessment of 4GS index score, the term “high 4GS index score” means the 4GS index score, or greater, that is found in a reference HCC tumor cell that is known to have high 4GS index score, wherein the 4GS index score is higher than that in the reference cells Hep3B or HepG2, e.g. HUH-6, HUH-7, or JHH-7 cells. Additional reference cells with 4GS index scores similar to the reference cells mentioned above may be used as reference cells in the methods of the invention described herein. For any of the methods described herein, a higher 4GS (or 4-gene) index score always means higher IGF-2/INSR expression and lower IGFBP3/IGFBP7 expression. Similarly, for any of the methods described herein, a lower 4GS (or 4-gene) index score always means lower IGF-2/INSR expression and higher IGFBP3/IGFBP7 expression.

In any of the methods described herein wherein a comparison to a reference or control cell level is indicated to ascertain whether a biomarker level or index score is high or low, the comparison need not be a direct side-by-side comparison of the sample with a reference cell, but may be a comparison of the sample biomarker level or index score with data from a previously determined level for a reference cell, under identical conditions. The reference cell may also be any cell that has the same or similar biomarker levels to the specific reference cells indicated herein as being suitable. It is the biomarker level of the reference cell that is important, rather than any particular cell itself that may be used for comparison.

“HepG2” tumor cells as used herein, refers to cells of the cell line HepG2, available from the American Tissue Culture Collection (ATCC) as HB-8065™, derived from a human HCC. The cell line was deposited by the Wistar Institute.

“Hep3B” tumor cells as used herein, refers to cells of the cell line Hep3B, available from the American Tissue Culture Collection (ATCC) as HB8064™, derived from a human HCC. The cell line was deposited by the Wistar Institute.

“HUH7” tumor cells as used herein, refers to cells of the cell line HuH-7, available from the Health Science Research Resources Bank (Japan) as JCRB No. JCRB0403, derived from a human hepatoma. The cell line was deposited by J. Sato.

“HUH1” tumor cells as used herein, refers to cells of the cell line HuH-1, available from the Health Science Research Resources Bank (Japan) as JCRB No. JCRB0199, derived from a human HCC. The cell line was deposited by N. Huh.

“HUH6” tumor cells as used herein, refers to cells of the cell line HUH6 Clone 5, available from the Health Science Research Resources Bank (Japan) as JCRB No. JCRB0401, derived from a human hepatoblastoma. The cell line was deposited by J. Sato.

“JHH-5” tumor cells as used herein, refers to cells of the cell line JHH-5, available from the Health Science Research Resources Bank (Japan) as JCRB No. JCRB 1029, derived from a human HCC. The cell line was deposited by S, Nagamori.

“JHH-7” tumor cells as used herein, refers to cells of the cell line JHH-7, available from the Health Science Research Resources Bank (Japan) as JCRB No. JCRB 1031, derived from a human HCC. The cell line was deposited by S, Nagamori.

“SK-HEP1” tumor cells as used herein, refers to cells of the cell line SK-HEP1, available from the American Tissue Culture Collection (ATCC) as HTB-52™, derived from a human adenocarcinoma. The cell line was deposited by G. Trempe and L. J. Old.

“SNU-182” tumor cells as used herein, refers to cells of the cell line SNU-182, available from the American Tissue Culture Collection (ATCC) as CRL²²³⁵™, derived from a human HCC. The cell line was deposited by J. Park.

“SNU-387” tumor cells as used herein, refers to cells of the cell line SNU-387, available from the American Tissue Culture Collection (ATCC) as CRL-2237™, derived from a human HCC. The cell line was deposited by J. Park.

“SNU-398” tumor cells as used herein, refers to cells of the cell line SNU-398, available from the American Tissue Culture Collection (ATCC) as CRL-2233™, derived from a human HCC. The cell line was deposited by J. Park.

“SNU-423” tumor cells as used herein, refers to cells of the cell line SNU-423, available from the American Tissue Culture Collection (ATCC) as CRL²²³⁸™, derived from a human HCC. The cell line was deposited by J. Park.

“SNU-449” tumor cells as used herein, refers to cells of the cell line SNU-449, available from the American Tissue Culture Collection (ATCC) as CRL²²³⁴™, derived from a human HCC. The cell line was deposited by J. Park.

“SNU-475” tumor cells as used herein, refers to cells of the cell line SNU-475, available from the American Tissue Culture Collection (ATCC) as CRL-2236™, derived from a human HCC. The cell line was deposited by J. Park.

“HLE” tumor cells as used herein, refers to cells of the cell line HLE, available from the Health Science Research Resources Bank (Japan) as JCRB No. JCRB0404, derived from a human HCC. The cell line was deposited by J. Sato.

“HLF” tumor cells as used herein, refers to cells of the cell line HLF, available from the Health Science Research Resources Bank (Japan) as JCRB No. JCRB0405, derived from a human HCC. The cell line was deposited by J. Sato.

“JHH-1” tumor cells as used herein, refers to cells of the cell line JHH-1, available from the Health Science Research Resources Bank (Japan) as JCRB No. JCRB 1062, derived from a human HCC. The cell line was deposited by S, Nagamori.

“JHH-4” tumor cells as used herein, refers to cells of the cell line JHH-4, available from the Health Science Research Resources Bank (Japan) as JCRB No. JCRB0435, derived from a human HCC. The cell line was deposited by S, Nagamori.

“JHH-6” tumor cells as used herein, refers to cells of the cell line JHH-6, available from the Health Science Research Resources Bank (Japan)) as JCRB No. JCRB1030, derived from a human HCC. The cell line was deposited by S, Nagamori.

“PLC/PRF/5” tumor cells as used herein, refers to cells of the cell line PLC/PRF/5, available from the American Tissue Culture Collection (ATCC) as CRL⁸⁰²⁴™, derived from a human hepatoma. The cell line was deposited by W. J. McAleer.

“JHH-2” tumor cells as used herein, refers to cells of the cell line JHH-2, available from the Health Science Research Resources Bank (Japan) as JCRB No. JCRB 1028, derived from a human HCC. The cell line was deposited by S, Nagamori.

In another embodiment of any of the methods described herein that involve assessment of a 4GS index score using, the term “high 4GS index score” means a 4GS index score of −4, or greater (e.g. −5, −6, −7, −8, −9, −9, −10, −11, −12, etc.), wherein gene expression is determined by RT-PCR, essentially as described herein. In another embodiment of any of the methods described herein that involve assessment of a 4GS index score, the term “low 4GS index score” means a 4GS index score of less than −4, (e.g. −3, −2, −1, 0, 1, 2, 3, 4, 5, etc.), wherein gene expression is determined by RT-PCR, essentially as described herein. One of skill in the art will appreciate that if a different method is used to determine gene expression levels, the magnitude and sign of the threshold between a high and low index score may differ. For example, determination of gene expression by gene array analysis (e.g. using Affymetrix chips) will produce index scores for OSI-906 sensitive HCC tumor cells that are positive rather than negative.

The present invention further provides a PCR primer set consisting of a pair of primers for each of the following genes: INSR, IGF2, IGFBP3 and IGFBP7. The present invention further provides a PCR primer set consisting of a pair of primers for each of the following genes: AFP, INSR, IGF2, IGFBP3 and IGFBP7. The present invention further provides a PCR primer set consisting of a pair of primers for each of the following genes: CDH1, AFP, INSR, IGF2, IGFBP3 and IGFBP7. The present invention further provides a PCR primer set consisting of a pair of primers for one or more of the following 20 genes: AFP, ACTN1, SPARC, ITGB3, PLAUR, CDH2, SNAIL SNAI2, TWIST1, VCAN, VIM, ZEB1, ZEB2, CDH1, CLDN3, ERBB3, MTA3, MAP7, TJP3 and OCLN, plus the genes INSR, IGF2, IGFBP7 and IGFBP3.

The present invention further provides a DNA microarray chip consisting of a solid surface and a probe set, said probe set consisting of probes specific for each of the following genes: INSR, IGF2, IGFBP3 and IGFBP7. The present invention further provides a DNA microarray chip consisting of a solid surface and a probe set, said probe set consisting of probes specific for each of the following genes s: AFP, INSR, IGF2, IGFBP3 and IGFBP7. The present invention further provides DNA microarray chip consisting of a solid surface and a probe set, said probe set consisting of probes specific for each of the following genes: CDH1, AFP, INSR, IGF2, IGFBP3 and IGFBP7.

The present invention further provides DNA microarray chip consisting of a solid surface and a probe set, said probe set consisting of probes specific for each of the following 20 genes: AFP, ACTN1, SPARC, ITGB3, PLAUR, CDH2, SNAI1, SNAI2, TWIST1, VCAN, VIM, ZEB1, ZEB2, CDH1, CLDN3, ERBB3, MTA3, MAP7, TJP3 and OCLN, plus the genes INSR, IGF2, IGFBP7 and IGFBP3.

The present invention further provides a method of identifying patients with hepatocellular carcinoma (HCC) who are most likely to benefit from treatment with an IGF-1R kinase inhibitor, comprising: obtaining a sample of a patient's HCC tumor; determining whether the HCC tumor cells express a high level of INSR; and identifying the patient as one most likely to benefit from treatment with an IGF-1R kinase inhibitor if the tumor cells express a high level of INSR. In one embodiment of this method a high level of INSR is a level equal to or greater than a level found in a reference HCC tumor cell. In one embodiment, the reference HCC tumor cell is JHH-2, or a cell with a similar level of INSR. In an alternative embodiment of this method a high level of INSR is a level equal to or greater than a level found in a non-HCC reference cell, e.g. a cell that has a similar level of INSR to JHH-2.

This invention also encompasses any of the methods of the invention described herein, but wherein the step of “obtaining a sample of a patient's HCC tumor” is not mandatory, and may be omitted. In such cases, the step of determining tumor biomarker expression may for example be performed on a previously processed tumor sample, e.g. a cell extract, an RNA preparation, a protein preparation, or the like, from which biomarker expression can be assessed, or a biological fluid where the tumor biomarker can be found, as an alternative to the tumor sample itself (e.g. a biopsy).

The present invention further provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a sample of the patient's tumor; determining whether the tumor cells express a high level of INSR; and identifying the patient as likely to benefit from treatment with an IGF-1R kinase inhibitor if the tumor cells express a high level of INSR, and (B) administering to said patient a therapeutically effective amount of an IGF-1R kinase inhibitor if the patient is diagnosed to be potentially responsive to an IGF-1R kinase inhibitor. In one embodiment of this method a high level of INSR is a level equal to or greater than a level found in a reference HCC tumor cell. In one embodiment, the reference HCC tumor cell is JHH-2, or a cell with a similar level of INSR. In an alternative embodiment of this method a high level of INSR is a level equal to or greater than a level found in a non-HCC reference cell, e.g. a cell that has a similar level of INSR to JHH-2.

The present invention further provides a method of identifying patients with hepatocellular carcinoma (HCC) who are most likely to benefit from treatment with an IGF-1R kinase inhibitor, comprising: obtaining a sample of a patient's HCC tumor; determining whether the HCC tumor cells express a high level of IGF2; and identifying the patient as one most likely to benefit from treatment with an IGF-1R kinase inhibitor if the tumor cells express a high level of IGF2. In one embodiment of this method a high level of IGF2 is a level equal to or greater than a level found in a reference HCC tumor cell. In one embodiment, the reference HCC tumor cell is HUH-6, or a cell with a similar level of IGF2. In an alternative embodiment of this method a high level of IGF2 is a level equal to or greater than a level found in a non-HCC reference cell, e.g. a cell that has a similar level of IGF2 to HUH-6.

The present invention further provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a sample of the patient's tumor; determining whether the tumor cells express a high level of IGF2; and identifying the patient as likely to benefit from treatment with an IGF-1R kinase inhibitor if the tumor cells express a high level of IGF2, and (B) administering to said patient a therapeutically effective amount of an IGF-1R kinase inhibitor if the patient is diagnosed to be potentially responsive to an IGF-1R kinase inhibitor. In one embodiment of this method a high level of IGF2 is a level equal to or greater than a level found in a reference HCC tumor cell. In one embodiment, the reference HCC tumor cell is HUH-6, or a cell with a similar level of IGF2. In an alternative embodiment of this method a high level of IGF2 is a level equal to or greater than a level found in a non-HCC reference cell, e.g. a cell that has a similar level of IGF2 to HUH-6.

The present invention further provides a method of identifying patients with hepatocellular carcinoma (HCC) who are most likely to benefit from treatment with an IGF-1R kinase inhibitor, comprising: obtaining a sample of a patient's HCC tumor; determining whether the HCC tumor cells express a high level of IGFBP3; and identifying the patient as one not likely to benefit from treatment with an IGF-1R kinase inhibitor if the tumor cells express a high level of IGFBP3. In one embodiment of this method a high level of IGFBP3 is a level equal to or greater than a level found in a reference HCC tumor cell. In one embodiment, the reference HCC tumor cell is PLC/PRF/5, or a cell with a similar level of IGFBP3. In an alternative embodiment of this method a high level of IGFBP3 is a level equal to or greater than a level found in a non-HCC reference cell, e.g. a cell that has a similar level of IGFBP3 to PLC/PRF/5.

The present invention further provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a sample of the patient's tumor; determining whether the tumor cells express a high level of IGFBP3; and identifying the patient as not likely to benefit from treatment with an IGF-1R kinase inhibitor if the tumor cells express a high level of IGFBP3, and (B) administering to said patient a therapeutically effective amount of an IGF-1R kinase inhibitor if the patient is diagnosed to be potentially responsive to an IGF-1R kinase inhibitor. In one embodiment of this method a high level of IGFBP3 is a level equal to or greater than a level found in a reference HCC tumor cell. In one embodiment, the reference HCC tumor cell is PLC/PRF/5, or a cell with a similar level of IGFBP3. In an alternative embodiment of this method a high level of IGFBP3 is a level equal to or greater than a level found in a non-HCC reference cell, e.g. a cell that has a similar level of IGFBP3 to PLC/PRF/5.

The present invention further provides a method of identifying patients with hepatocellular carcinoma (HCC) who are most likely to benefit from treatment with an IGF-1R kinase inhibitor, comprising: obtaining a sample of a patient's HCC tumor; determining whether the HCC tumor cells express a high level of IGFBP7; and identifying the patient as one not likely to benefit from treatment with an IGF-1R kinase inhibitor if the tumor cells express a high level of IGFBP7. In one embodiment of this method a high level of IGFBP7 is a level equal to or greater than a level found in a reference HCC tumor cell. In one embodiment, the reference HCC tumor cell is JHH-4, or a cell with a similar level of IGFBP7. In an alternative embodiment of this method a high level of IGFBP7 is a level equal to or greater than a level found in a non-HCC reference cell, e.g. a cell that has a similar level of IGFBP7 to JHH-4.

The present invention further provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a sample of the patient's tumor; determining whether the tumor cells express a high level of IGFBP7; and identifying the patient as not likely to benefit from treatment with an IGF-1R kinase inhibitor if the tumor cells express a high level of IGFBP7, and (B) administering to said patient a therapeutically effective amount of an IGF-1R kinase inhibitor if the patient is diagnosed to be potentially responsive to an IGF-1R kinase inhibitor.

In one embodiment of this method a high level of IGFBP7 is a level equal to or greater than a level found in a reference HCC tumor cell. In one embodiment, the reference HCC tumor cell is JHH-4, or a cell with a similar level of IGFBP7. In an alternative embodiment of this method a high level of IGFBP7 is a level equal to or greater than a level found in a non-HCC reference cell, e.g. a cell that has a similar level of IGFBP7 to JHH-4.

The present invention further provides any of the methods described herein where INSR, IGF2, IGFBP3 or IGFBP7 expression level is used to determine if a patient is likely to benefit from treatment with an IGF-1R kinase inhibitor wherein a high level is a level above a defined threshold as determined by a threshold determination analysis. In one embodiment, the threshold determination analysis comprises a receiver operator characteristic curve analysis.

The present invention provides for any of the methods of identifying patients with HCC cancer who are most likely to benefit from treatment with an IGF-1R kinase inhibitor described herein involving determination of AFP expression level (in the tumor, or serum, blood, or plasma levels), the method as described but including an additional step of assessment of the level of IGF-1 and/or IGF-2 (i.e. insulin-like growth factors 1 and/or 2) in the HCC tumor of the patient, wherein the presence of IGF-1 and/or IGF-2 indicates that an activating ligand for IGF-1R is present. The present invention also provides for any of the methods of treatment with an IGF-1R kinase inhibitor described herein, the method as described but including prior to the step of administering to the patient an IGF-1R kinase inhibitor, an additional step of assessment of the level of IGF-1 and/or IGF-2 (i.e. insulin-like growth factors 1 and/or 2) in the HCC tumor of the patient. Since IGF-1R has been reported to be activated only upon ligand (i.e. IGF-1 and/or IGF-2) binding, if there is no IGF-1R ligand present in a tumor, then even if one or more of the methods of the instant invention predict that it should be sensitive to inhibition by IGF-1R kinase inhibitors, the tumor cells under such circumstances be not be relying on the IGF-1R signaling pathway for growth and survival, and thus an IGF-1R kinase inhibitor would probably not be an effective treatment. Many tumors have been found to express elevated levels of IGF-1 and/or IGF-2 (Pollack, M. N. et al. (2004) Nature Reviews Cancer 4:505-518), which could originate from the tumor cells themselves, from stromal cells present in the tumor, or via the vascular system from non-tumor cells (e.g. liver cells). Assessment of the level of IGF-1 and/or IGF-2 can be performed by any method known in the art, such as for example any of the methods described herein for assessment of biomarkers levels, e.g. immunoassay determination of IGF-1 and/or IGF-2 protein levels; determination of IGF-1 and/or IGF-2 mRNA transcript levels. In an alternative embodiment, the of step of assessment of the level of IGF-1 and/or IGF-2 (i.e. insulin-like growth factors 1 and/or 2) in the tumor of the patient can be replaced with a step of assessment of the level of IGF-1 and/or IGF-2 (i.e. insulin-like growth factors 1 and/or 2) in the blood or serum of the patient. This alternative, though not a direct measure of the level of IGF-1 and/or IGF-2 in the tumor, can give an indication of the potential availability of ligand to the IGF-1R in the tumor, and is a simpler and less expensive test. The potential disadvantage of this indirect assessment of IGF-1 and/or IGF-2 is that it may not give a true indication of the levels of ligand in the tumor if IGF-1 and/or IGF-2 is produced locally in the tumor, either by the tumor cells themselves, or by stromal cells within the tumor. In these methods with the additional step of assessment of the level of IGF-1 and/or IGF-2, the presence of IGF-1 and/or IGF-2 may be made an additional condition required for identifying the patient as likely to benefit from treatment with an IGF-1R kinase inhibitor, or to be diagnosed to be potentially responsive to an IGF-1R kinase inhibitor, and thus required prior to administering to said patient a therapeutically effective amount of an IGF-1R kinase inhibitor.

Accordingly, the invention provides a method of identifying patients with hepatocellular carcinoma who are most likely to benefit from treatment with an IGF-1R kinase inhibitor, comprising: obtaining a sample of a patient's tumor; determining whether the tumor cells express a high level of AFP; assessing whether IGF-1 and/or IGF-2 is present in the tumor; and identifying the patient as one most likely to benefit from treatment with an IGF-1R kinase inhibitor if the tumor cells express a high level of AFP and IGF-1 and/or IGF-2 is present.

The invention also provides a method for treating hepatocellular carcinomas (HCC) or HCC tumor metastases in a patient, comprising the steps of: assessing a patient's likely responsiveness to an IGF-1R kinase inhibitor, by determining the presence or absence of high expression levels of AFP in the HCC tumor cells, wherein the presence of high AFP expression correlates with high sensitivity to inhibition by IGF-1R kinase inhibitors; assessing the level of IGF-1 and/or IGF-2 in the tumor (or blood or serum) of the patient; and administering to said patient a therapeutically effective amount of an IGF-1R kinase inhibitor if the patient is diagnosed to be potentially responsive to an IGF-1R kinase inhibitor, and IGF-1 and/or IGF-2 is determined to be present in the tumor (or blood or serum levels indicate the potential availability of IGF-1 and/or IGF-2 to the tumor cells). In one embodiment the presence of IGF-1 and/or IGF-2 in the HCC tumor is determined by assessing the level of IGF-1 and/or IGF-2 protein in the tumor cells (e.g. by immunohistochemistry). In another embodiment the presence of IGF-1 and/or IGF-2 in the HCC tumor is determined by assessing the level of IGF-1 and/or IGF-2 RNA transcripts in the tumor cells (e.g. by quantitative RT-PCR).

The invention also provides a method for treating hepatocellular carcinomas (HCC) or HCC tumor metastases in a patient, comprising the steps of: assessing a patient's likely responsiveness to an IGF-1R kinase inhibitor, by determining whether the HCC tumor cells express a high level of AFP and assessing whether IGF-1 and/or IGF-2 is present in the tumor; and administering to said patient a therapeutically effective amount of an IGF-1R kinase inhibitor if the patient is diagnosed to be potentially responsive to an IGF-1R kinase inhibitor by having tumor cells that posess a high expression level of AFP and the presence of IGF-1 and/or IGF-2 in the tumor.

The invention also provides a method of identifying patients with hepatocellular carcinoma who are most likely to benefit from treatment with an IGF-1R kinase inhibitor, comprising: obtaining a sample of a patient's tumor; determining whether the tumor cells express a high level of AFP; assessing whether IGF-1 and/or IGF-2 is present in the tumor; and identifying the patient as one most likely to benefit from treatment with an IGF-1R kinase inhibitor if the tumor cells express a high level of AFP, and IGF-1 and/or IGF-2 is present in the tumor.

The invention also provides a method for treating hepatocellular carcinoma in a patient, comprising the steps of: (A) assessing a patient's likely responsiveness to an IGF-1R kinase inhibitor by determining if the patient has an hepatocellular carcinoma that is likely to respond to treatment with an IGF-1R kinase inhibitor by: obtaining a sample of the patient's tumor; determining whether the tumor cells express a high level of AFP and assessing whether IGF-1 and/or IGF-2 is present in ther tumor; and identifying the patient as likely to benefit from treatment with an IGF-1R kinase inhibitor if the tumor cells express a high level of AFP, and IGF-1 and/or IGF-2 is present in the tumor, and (B) administering to said patient a therapeutically effective amount of an IGF-1R kinase inhibitor if the patient is diagnosed to be potentially responsive to an IGF-1R kinase inhibitor by having tumor cells that possess a high expression level of AFP, and the presence of IGF-1 and/or IGF-2 in the tumor.

The effectiveness of treatment in the methods described herein can be determined for example by measuring the decrease in size of the hepatocellular carcinoma present in the patients, or a biomarker that correlates with the presence of hepatocellular carcinoma cells, or by assaying a molecular determinant of the degree of proliferation of the hepatocellular carcinoma cells.

Although the experimental examples provided herein involve the IGF-1R kinase inhibitor, OSI-906, the methods of the present invention are not limited to the prediction of patients or tumors that will respond or not respond to any particular IGF-1R kinase inhibitor, but rather, can be used to predict patient's outcome to any IGF-1R kinase inhibitor, including IGF-1R kinase inhibitors that inhibit both IGF-1R and IR kinases (e.g. OSI-906 (OSI Pharmaceuticals, Inc.), BMS-554417 (Haluska P, et al. Cancer Res 2006; 66(1):362-71); BMS 536924 (Huang, F. et al. (2009) Cancer Res. 69(1):161-170), BMS-754807 (Bristol-Myers Squibb)), inhibitors that are small molecules (e.g. AXL-1717 (Axelar AB), XL-228 (Exelixus), INSM-18 (Insured Inc.)), peptides, antibodies (e.g. IMCL-A12 (a.k.a. cixutumumab; Imclone), MK-0646 (Merck), CP-751871(a.k.a. figitumab; Pfizer), AMG-479 (Amgen), SCH-717454 (a.k.a. robatumumab; Schering-Plough/Merck), antibody fragments, nucleic acids, or other types of IGF-1R kinase inhibitor inhibitors. Similarly, the methods of treatment with an IGF-1R kinase inhibitor described herein may use any of these types of IGF-1R kinase inhibitor. Furthermore, in another embodiment of any of the methods described herein the IGF-1R kinase inhibitor may be an IGF-1R kinase inhibitor approved by a government regulatory authority (e.g. US Food and Drug Administration (FDA); European Medicines Agency; Japanese Ministry of Health, Labour & Welfare; UK Medicines and Healthcare Products Regulatory Agency (MHRA)) (e.g. any of the IGF-1R kinase inhibitors disclosed herein that have been so approved).

In any of the methods, compositions or kits of the invention described herein, the term “small molecule IGF-1R kinase inhibitor” refers to a low molecular weight (i.e. less than 5000 Daltons; preferably less than 1000, and more preferably between 300 and 700 Daltons) organic compound that inhibits IGF-1R kinase by binding to the kinase domain of the enzyme. Examples of such compounds include IGF-1R kinase inhibitors of Formula (I) as described herein. The IGF-1R kinase inhibitor of Formula (I) can be any IGF-1R kinase inhibitor compound encompassed by Formula (I) that inhibits IGF-1R kinase upon administration to a patient. Examples of such inhibitors have been published in US Published Patent Application US 2006/0235031, which is incorporated herein in its entirety, and include OSI-906 (cis-3-[8-amino-1-(2-phenyl-quinolin-7-yl)-imidazo[1,5-a]pyrazin-3-yl]-1-methyl-cyclobutanol), as used in the experiments described herein.

One of skill in the medical arts, particularly pertaining to the application of diagnostic tests and treatment with therapeutics, will recognize that biological systems are somewhat variable and not always entirely predictable, and thus many good diagnostic tests or therapeutics are occasionally ineffective. Thus, it is ultimately up to the judgement of the attending physician to determine the most appropriate course of treatment for an individual patient, based upon test results, patient condition and history, and his own experience. There may even be occasions, for example, when a physician will choose to treat a patient with an IGF-1R kinase inhibitor even when a tumor is not predicted to be particularly sensitive to IGF-1R kinase inhibitors, based on data from diagnostic tests or from other criteria, particularly if all or most of the other obvious treatment options have failed, or if some synergy is anticipated when given with another treatment. The fact that the IGF-1R kinase inhibitors as a class of compounds are relatively well tolerated compared to many other anti-cancer compounds, such as more traditional chemotherapy or cytotoxic agents used in the treatment of cancer, makes this a more viable option. Also, it should be noted that while the biomarkers disclosed herein predict which patients with tumors are likely to receive the most benefit from IGF-1R kinase inhibitors, it does not necessarily mean that patients with tumors which do not possess a biomarker predicting sensitivity will receive no benefit, just that a more modest effect is to be anticipated.

As described herein, this invention provides methods for using the expression or level of various biomarkers to predict tumor sensitivity to inhibition by IGF-1R kinase inhibitors. All diagnostic methods have potential advantages and disadvantages, and while the preferred method will ultimately depend on individual patient circumstances, the use of multiple diagnostic methods will likely improve one's ability to accurately predict the likely outcome of a therapeutic regimen comprising use of an IGF-1R kinase inhibitor. Therefore, this invention also provides methods for diagnosing or for treating a patient with HCC cancer, comprising the use of two or more diagnostic methods for predicting sensitivity to inhibition by IGF-1R kinase inhibitors, followed in the case of a treatment method by administering to said patient of a therapeutically effective amount of an IGF-1R kinase inhibitor if two or more of the diagnostic methods indicate that the patient is potentially responsive to an IGF-1R kinase inhibitor. One of the diagnostic methods for predicting sensitivity to inhibition by IGF-1R kinase inhibitors may be a method as described herein using HCC AFP expression level, AFP serum (or blood, or plasma) level, the degree of expression of IR, IGF-2, IGFBP3 or IGFBP7 in the HCC tumor cells, or the value of a 4-gene index calculated using the HCC expression values for each of these four genes. The other diagnostic method(s) may be any method known in the art for using biomarkers to predict sensitivity to inhibition by IGF-1R kinase inhibitors that is found to be applicable to HCC, e.g. biomarkers predicting sensitivity or resistance to IGF-1R kinase inhibitors as described in T. Pitts et al. (2009) EORTC Conference, Boston, Mass., abstract #2141, or Pitts, T. M. et al. (2010) Clin. Can. Res. 16(12):3193-3204; pERK, pHER3 or HER3 (US 2009/0093488); IGF-1; or other biomarkers reported to predict sensitivity to IGF-1R kinase inhibitors (e.g. see Huang F. H. W., et al. Identification of sensitivity markers for BMS-536924, an inhibitor for insulin-like growth factor-1 receptor. J Clin Oncol ASCO Ann Meet Proc Part 12007; 25:3506; or U.S. patent applications 61/310,031 and 61/310,038).

In many of the methods of this invention, the expression level of a tumor cell gene is assessed by assaying a tumor biopsy. However, in an alternative embodiment, expression level of the tumor cell genes can be assessed in bodily fluids or excretions containing detectable levels of tumor cells originating from the tumor. Bodily fluids or excretions useful in the present invention include blood, urine, saliva, stool, pleural fluid, lymphatic fluid, sputum, ascites, prostatic fluid, cerebrospinal fluid (CSF), or any other bodily secretion or derivative thereof. By blood it is meant to include whole blood, plasma, serum or any derivative of blood. Assessment of tumor cell genes in such bodily fluids or excretions can sometimes be preferred in circumstances where an invasive sampling method is inappropriate or inconvenient. For assessment of tumor cell gene expression, patient samples containing tumor cells, or proteins or nucleic acids produced by these tumor cells, may be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing gene expression in the sample. Likewise, tumor biopsies may also be subjected to post-collection preparative and storage techniques, e.g., fixation. Microdissection and/or microdisection methods (e.g. Laser Microdissection and Pressure Catapulting (LMPC), for example, using the PALM® Micro Beam microscope (P.A.L.M. Microlaser Technologies AG, Bernried, Germany); SL-Microtest UV laser microdissection system (Molecular Machines & Industries, Glattbrugg, Switzerland)) may be used to enrich the tumor cell population of a tumor sample by removing normal tissue cells or stromal cells (e.g. de Bruin E C. et al. BMC Genomics. 2005 Oct. 14; 6:142; Dhal, E. et al. Clinical Cancer Research July 2006 12; 3950; Funel, N. et al. Laboratory Investigation (2008) 88:773-784, doi:10.1038/1abinvest.2008.40, published online 19 May 2008). Primary tumor cell cultures may also be prepared in order to produce a pure tumor cell population.

In the methods of this invention, assessment of biomarker status of tumor cells can be based on any of a number of well-established molecular assays known in the art which have been found to be sufficiently sensitive, specific, and reliable. Many molecular diagnostic laboratories exist to which a sample of a tumor can be sent for biomarker status analysis. The sample can be fresh, frozen or paraffin-embedded tissue depending on the methodology used. Preferably, a pathologist should confirm that a tissue specimen contains cancer cells and estimate the content of tumor cells (percentage tumor nuclei out of all nuclei present) in the specimen. This estimation of tumor cell content can be important since certain biomarker assays have different analytical sensitivities and an attempt should be made to enrich to a level that is acceptable for the assay being used.

Commercial AFP serum (or blood, or plasma) assays may be employed in the practice of the methods of this invention. e.g. the Beckman Coulter Access alpha-fetoprotein (AFP) immunoassay; the auto DELFIA AFP fluorometric immunoassay kit (DELFIA, Wallac, Finland); AFP Enzyme Immunoassay (Diagnostic Automation, Inc., Calabasas, Calif.).

In the methods of this invention wherein high (or low) levels of a biomarker correlate with the sensitivity of hepatocellular carcinoma cell growth to inhibition by an IGF-1R kinase inhibitor, methods known in the art for determining defined threshold levels for patient response may be employed. Thus, for example, if the biomarker level is above the defined threshold, this may indicate that the tumor is likely to be responsive to an IGF-1R kinase inhibitor, and if below said threshold, it may be non-responsive to an IGF-1R kinase inhibitor. Thus the defined threshold can be used as a cutoff value to define what are high levels, and what are low levels of the biomarker. Thus in any of the methods of this invention a high level of a biomarker (e.g. HCC cell AFP, serum (or blood, or plasma) AFP, 4-gene index score, HCC cell expression level of INSR, IGF2, IGFBP3 or IGFBP7, expression of epithelial (e.g. E-cadherin) or mesenchymal genes in HCC tumor cells) may be a level above a defined threshold as determined by a threshold determination analysis, wherein the threshold determination analysis may comprise a receiver operator characteristic curve analysis. Similarly, in any of the methods of this invention a low level of a biomarker (e.g. HCC cell AFP, serum (or blood, or plasma) AFP, 4-gene index score, HCC cell expression level of INSR, IGF2, IGFBP3 or IGFBP7, expression of epithelial (e.g. E-cadherin) or mesenchymal genes HCC tumor cells) may be a level at or below a defined threshold as determined by a threshold determination analysis, wherein the threshold determination analysis may comprise a receiver operator characteristic curve analysis.

In another emdodiment of methods described herein, for AFP expression in HCC tumor cells or AFP serum (or blood, or plasma) level, expression of IR or IGF-2 in HCC tumor cells, expression of epithelial (e.g. E-cadherin) or mesenchymal genes in HCC tumor cells, or the value of a 4-gene index calculated using the HCC expression values for the genes IR, IGF-2, IGFBP3 and IGFBP7, a value or level above a defined threshold indicates that the tumor is likely to be responsive to an IGF-1R kinase inhibitor, and a value or level below a defined threshold indicates that the tumor is likely to be non-responsive to an IGF-1R kinase inhibitor. The threshold value may be determined, for example, as described herein, using an ROC curve analysis, or by any comparable statistical methods.

In another emdodiment, for expression of IGFBP3 or IGFBP7 in HCC tumor cells, a value or level above a defined threshold indicates that the tumor is likely to be non-responsive to an IGF-1R kinase inhibitor, and a value or level below a defined threshold indicates that the tumor is likely to be responsive to an IGF-1R kinase inhibitor. The threshold value may be determined, for example, as described herein, using an ROC curve analysis, or by any comparable statistical methods.

It is contemplated that a given threshold value may vary depending on the patient population. For any given patient population, an optimum threshold value can be determined (or at least approximated) empirically by performing a threshold determination analysis. In many effective methods, threshold determination analysis includes receiver operator characteristic (ROC) curve analysis.

ROC curve analysis is an established statistical technique, the application of which is within ordinary skill in the art. For a discussion of ROC curve analysis, see generally Zweig et al., 1993, “Receiver operating characteristic (ROC) plots: a fundamental evaluation tool in clinical medicine,” Clin. Chem. 39:561-577; and Pepe, 2003, The statistical evaluation of medical tests for classification and prediction, Oxford Press, New York.

Gene index scores and expression values, and optimum threshold values, may vary from one patient population to another. Therefore, a threshold determination analysis preferably is performed on one or more datasets representing any given patient population to be tested using the present invention. The dataset used for threshold determination analysis includes: (a) actual response data (response or non-response), and (b) a gene index score or biomarker expression level for each tumor sample from a group of human tumors or animal tumors. Once a threshold value is determined with respect to a given patient population, that threshold can be applied to interpret gene index scores or biomarker expression levels from HCC tumors of that patient population.

The ROC curve analysis is performed essentially as follows. For AFP expression in HCC tumor cells or AFP serum (or blood, or plasma) level, expression of IR or IGF-2 in HCC tumor cells, or the value of a 4-gene index calculated using the HCC expression values for the genes IR, IGF-2, IGFBP3 and IGFBP7, any sample with a gene index score or biomarker expression level greater than threshold is identified as a responder, and any sample with a gene index score or biomarker expression level less than or equal to threshold is identified as non-responder. For every gene index score or biomarker expression level from a tested set of samples, “responders” and “non-responders” (hypothetical calls) are classified using that gene index score or biomarker expression level as the threshold. This process enables calculation of TPR (“true positive rate”; y vector) and FPR (“false positive rate”; x vector) for each potential threshold, through comparison of hypothetical calls against the actual response data for the data set. Then an ROC curve is constructed by making a dot plot, using the TPR vector, and FPR vector. If the ROC curve is above the diagonal from (0, 0) point to (1.0, 0.5) point, it shows that the a gene index score or biomarker expression level test result is a better test than random. For expression of IGFBP3 or IGFBP7 in HCC tumor cells, an essentially identical process is followed, except that the non-respondes are above the threshold, and the responders are below the threshold.

The ROC curve can be used to identify the best operating point. The best operating point is the one that yields the best balance between the cost of false positives weighed against the cost of false negatives. These costs need not be equal. The average expected cost of classification at point x,y in the ROC space is denoted by the expression C=(1−p)alpha*x+p*beta(1−y) wherein: alpha=cost of a false positive, beta=cost of missing a positive (false negative), and p=proportion of positive cases.

False positives and false negatives can be weighted differently by assigning different values for alpha and beta. For example, if it is decided to include more patients in the responder group at the cost of treating more patients who are non-responders, one can put more weight on alpha. In this case, it is assumed that the cost of false positive and false negative is the same (alpha equals to beta). Therefore, the average expected cost of classification at point x,y in the ROC space is: C′=(1−p)*x+p*(1−y). The smallest C′ can be calculated after using all pairs of false positive and false negative (x, y). The optimum gene index score or biomarker expression level threshold is calculated as the gene index score or biomarker expression level of the (x, y) at C′.

In any of the methods of this invention a high level of expression of a biomarker, wherein the biomarker is HCC cell AFP, 4-GS index score, CDH1 (i.e. E-Cadherin) or ERBB3, may be a level equal to or greater than a level found in a reference HCC tumor cell that has high sensitivity to growth inhibition by an IGF-1R kinase inhibitor, e.g. Hep3B or HUH1 for AFP; Hep3B or HepG2 for 4-GS index score; HUH1 or HUH7 for CDH1 (i.e. E-Cadherin); HUH1, HUH6 or HUH7 for ERBB3.

In any of the methods of this invention a low level of expression of a biomarker, wherein the biomarker is HCC cell AFP, 4-GS index score, CDH1 (i.e. E-Cadherin) or ERBB3, may be a level that is lower than a level found in a reference HCC tumor cell that has high sensitivity to growth inhibition by an IGF-1R kinase inhibitor, e.g. Hep3B or HUH1 for AFP; Hep3B or HepG2 for 4-GS index score; HUH1 or HUH7 for CDH1 (i.e. E-Cadherin); HUH1, HUH6 or HUH7 for ERBB3.

In the methods of this invention, gene expression in a tumor cell can be assessed by using any of the standard bioassay procedures known in the art for determination of the level of expression of a gene, including for example immunohistochemistry (1HC), enzyme-linked immunosorbent assay (ELISA), radioimmunoassay (RIA), immunoprecipitation, immunoblotting, immunofluorescence microscopy, real-time polymerase chain reaction (RT-PCR), in situ hybridization, cDNA microarray, in vitro transcription, or the like, as described in more detail below.

A general principle of diagnostic assays as described herein involves preparing a sample or reaction mixture that may contain an expressed gene product, and a probe, under appropriate conditions and for a time sufficient to allow the product and probe to interact and bind, thus forming a complex that can be removed and/or detected in the reaction mixture or sample. These assays can be conducted in a variety of ways. For example, one method to conduct such an assay would involve anchoring the expressed product or probe onto a solid phase support, also referred to as a substrate, and detecting target product/probe complexes anchored on the solid phase at the end of the reaction. In one embodiment of such a method, a sample from a subject, which is to be assayed for presence and/or concentration of a gene product, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, biomarker or probe molecules which are immobilized through conjugation of biotin and streptavidin. Such biotinylated assay components can be prepared from biotin-NHS(N-hydroxy-succinimide) using techniques known in the art (e.g., biotinylation kit, Pierce Chemicals, Rockford, Ill.), and immobilized in the wells of streptavidin-coated microtiter plates (e.g. 96, 384, or 1536 well plates). In certain embodiments, the surfaces with immobilized assay components can be prepared in advance and stored.

Other suitable carriers or solid phase supports for such assays include any material capable of binding the class of molecule to which the biomarker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of expressed product/probe complexes anchored to the solid phase can be accomplished in a number of methods outlined herein.

In one embodiment, the probe, when it is the unanchored assay component, can be labeled for the purpose of detection and readout of the assay, either directly or indirectly, with detectable labels discussed herein and which are well-known to one skilled in the art.

It is also possible to directly detect product/probe complex formation without further manipulation or labeling of either component (biomarker or probe), for example by utilizing the technique of fluorescence energy transfer (i.e. FET, see for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a biomarker can be accomplished without labeling either assay component (probe or biomarker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In a particular embodiment, the level of mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA encoding a biomarker of the present invention. Other suitable probes for use in the diagnostic assays of the invention are described herein. Hybridization of an mRNA with the probe indicates that the biomarker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an AFFYMETRIX® gene chip array. A skilled artisan can readily adapt known mRNA detection methods for use in detecting the level of mRNA encoded by the biomarkers of the present invention.

An alternative method for determining the level of mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA that encodes the biomarker.

A tissue sample from a tumor in a human patient or an animal model can be used as a source of RNA so that the gene expression levels in the sample can be determined in accordance with the present invention. Generally, the tumor will be a carcinoma. The tissue sample can be obtained by using conventional tumor biopsy instruments and procedures. Endoscopic biopsy, excisional biopsy, incisional biopsy, fine needle biopsy or aspiration (FNA), core biopsy, punch biopsy, shave biopsy and skin biopsy are examples of recognized medical procedures that can be used by one of skill in the art to obtain tumor samples for use in practicing the invention. The tumor tissue sample should be large enough to provide sufficient RNA for measuring individual gene expression levels. The tumor tissue sample can be in any form that allows gene expression analysis, e.g., RNA extraction and quantitation. Accordingly, the tissue sample can be fresh, preserved through suitable cryogenic techniques, or preserved through non-cryogenic techniques. A standard process for handling clinical biopsy specimens is to fix the tissue sample in formalin and then embed it in paraffin. Samples in this form are commonly known as formalin-fixed, paraffin-embedded (FFPE) tissue. Suitable techniques of tissue preparation and tissue preservation for subsequent RNA extraction are well-known to those of skill in the art.

Individual gene expression levels for each gene are the input values used to calculate the 4-gene index value as described herein. Once a tissue sample is obtained it is necessary to determine, i.e., measure, the expression levels of the individual genes. Gene expression level can be determined by any suitable method. Two exemplary methods for measuring individual expression are DNA microarray analysis and qRT-PCR, which are discussed below. A prerequisite for either of these alternative methods is RNA isolation.

Methods for rapid and efficient extraction of eukaryotic mRNA, i.e., poly(a) RNA, from tissue samples or cultured cells are well established and known to those of skill in the art. See, e.g., Ausubel et al., 1997, Current Protocols of Molecular Biology, John Wiley & Sons. The tissue sample can be fresh, frozen or fixed paraffin-embedded (FFPE) clinical study tumor specimens. In general, RNA isolated from fresh or frozen tissue samples tends to be less fragmented than RNA from FFPE samples. FFPE samples of tumor material, however, are more readily available, and FFPE samples are suitable sources of RNA for use in methods of the present invention. For a discussion of FFPE samples as sources of RNA for gene expression profiling by RT-PCR, see, e.g., Clark-Langone et al., 2007, BMC Genomics 8:279. Also see, De Andres et al., 1995, Biotechniques 18:42044; and Baker et al., U.S. Patent Application Publication No. 2005/0095634. The use of commercially available kits with vendor's instructions for RNA extraction and preparation is widespread and common Commercial vendors of various RNA isolation products and complete kits include Qiagen (Valencia, Calif.), Invitrogen (Carlsbad, Calif.), Ambion (Austin, Tex.) and Exiqon (Woburn, Mass.).

In general, RNA isolation begins with tissue/cell disruption. During tissue/cell disruption it is desirable to minimize RNA degradation by RNases. One approach to limiting RNase activity during the RNA isolation process is to ensure that a denaturant is in contact with cellular contents as soon as the cells are disrupted. Another common practice is to include one or more proteases in the RNA isolation process. Optionally, fresh tissue samples are immersed in an RNA stabilization solution, at room temperature, as soon as they are collected. The stabilization solution rapidly permeates the cells, stabilizing the RNA for storage at 4° C., for subsequent isolation. One such stabilization solution is available commercially as RNALATER®. (Ambion, Austin, Tex.).

In some protocols, total RNA is isolated from disrupted tumor material by cesium chloride density gradient centrifugation. In general, mRNA makes up approximately 1% to 5% of total cellular RNA. Immobilized Oligo(dT), e.g., oligo(dT) cellulose, is commonly used to separate mRNA from ribosomal RNA and transfer RNA. If stored after isolation, RNA must be stored in under RNase-free conditions. Methods for stable storage of isolated RNA are known in the art. Various commercial products for stable storage of RNA are available.

The mRNA expression level for multiple genes can be measured using conventional DNA microarray expression profiling technology. A DNA microarray is a collection of specific DNA segments or probes affixed to a solid surface or substrate such as glass, plastic or silicon, with each specific DNA segment occupying a known location in the array. Hybridization with a sample of labeled RNA, usually under stringent hybridization conditions, allows detection and quantitation of RNA molecules corresponding to each probe in the array. After stringent washing to remove non-specifically bound sample material, the microarray is scanned by confocal laser microscopy or other suitable detection method. Modern commercial DNA microarrays, often known as DNA chips, typically contain tens of thousands of probes, and thus can measure expression of tens of thousands of genes simultaneously. Such microarrays can be used in practicing the present invention. Alternatively, custom chips containing as few probes as those needed to measure expression of the genes of interest (e.g. those that contribute to the 4-gene index described herein), plus necessary controls or standards (for data normalization, etc.), can be used in practicing the invention.

To facilitate data normalization, a two-color microarray reader can be used. In a two-color (two-channel) system, samples are labeled with a first fluorophore that emits at a first wavelength, while an RNA or cDNA standard is labeled with a second fluorophore that emits at a different wavelength. For example, Cy3 (570 nm) and Cy5 (670 nm) often are employed together in two-color microarray systems.

DNA microarray technology is well-developed, commercially available, and widely employed. Therefore, in performing methods of the invention, a person of ordinary skill in the art can use microarray technology to measure expression levels of genes without undue experimentation. DNA microarray chips, reagents (such as those for RNA or cDNA preparation, RNA or cDNA labeling, hybridization and washing solutions), instruments (such as microarray readers) and protocols are well known in the art and available from various commercial sources. Commercial vendors of microarray systems include Agilent Technologies (Santa Clara, Calif.) and Affymetrix (Santa Clara, Calif.), but other systems may be used.

The level of mRNA representing individual genes can be measured using conventional quantitative reverse transcriptase polymerase chain reaction (qRT-PCR) technology. Advantages of qRT-PCR include sensitivity, flexibility, quantitative accuracy, and ability to discriminate between closely related mRNAs. Guidance concerning the processing of tissue samples for quantitative PCR is available from various sources, including manufacturers and vendors of commercial products for qRT-PCR (e.g., Qiagen (Valencia, Calif.) and Ambion (Austin, Tex.)). Instrument systems for automated performance of qRT-PCR are commercially available and used routinely in many laboratories. An example of a well-known commercial system is the Applied Biosystems 7900HT Fast Real-Time PCR System (Applied Biosystems, Foster City, Calif.).

Once isolated mRNA is in hand, the first step in gene expression profiling by RT-PCR is the reverse transcription of the mRNA template into cDNA, which is then exponentially amplified in a PCR reaction. Two commonly used reverse transcriptases are avilo myeloblastosis virus reverse transcriptase (AMV-RT) and Moloney murine leukemia virus reverse transcriptase (MMLV-RT). The reverse transcription reaction typically is primed with specific primers, random hexamers, or oligo(dT) primers. Suitable primers are commercially available, e.g., GENEAMP® RNA PCR kit (Perkin Elmer, Waltham, Mass.). The resulting cDNA product can be used as a template in the subsequent polymerase chain reaction.

The PCR step is carried out using a thermostable DNA-dependent DNA polymerase. The polymerase most commonly used in PCR systems is a Thermus aquaticus (Taq) polymerase. The selectivity of PCR results from the use of primers that are complementary to the DNA region targeted for amplification, i.e., regions of the cDNAs reverse transcribed from the genes. Therefore, when qRT-PCR is employed in the present invention, primers specific to each gene are based on the cDNA sequence of the gene. Commercial technologies such as SYBR® green or TAQMAN® (Applied Biosystems, Foster City, Calif.) can be used in accordance with the vendor's instructions. Messenger RNA levels can be normalized for differences in loading among samples by comparing the levels of housekeeping genes such as beta-actin or GAPDH. The level of mRNA expression can be expressed relative to any single control sample such as mRNA from normal, non-tumor tissue or cells. Alternatively, it can be expressed relative to mRNA from a pool of tumor samples, or tumor cell lines, or from a commercially available set of control mRNA.

Suitable primer sets for PCR analysis of expression levels of genes can be designed and synthesized by one of skill in the art, without undue experimentation. Alternatively, complete PCR primer sets for practicing the present invention can be purchased from commercial sources, e.g., Applied Biosystems, based on the identities of the genes described. PCR primers preferably are about 17 to 25 nucleotides in length. Primers can be designed to have a particular melting temperature (Tm), using conventional algorithms for Tm estimation. Software for primer design and Tm estimation are available commercially, e.g., PRIMER EXPRESS™ (Applied Biosystems), and also are available on the internet, e.g., Primer3 (Massachusetts Institute of Technology). By applying established principles of PCR primer design, a large number of different primers can be used to measure the expression level of any given gene. Accordingly, the invention is not limited with respect to which particular primers are used for any given gene.

In another embodiment of the present invention, an expressed protein is detected. A preferred agent for detecting an expressed protein in the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to a probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells, or biological fluids in contact therewith (e.g. blood), express a biomarker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type Immune specific binding pairs are exemplified by antigen-antibody systems or hapten/anti-hapten systems, e.g. fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al, J Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

The invention also encompasses kits for detecting the expression of genes (e.g. genes of the 4-gene index described herein) in a biological sample. Such kits can be used to determine if a subject is suffering from a tumor that is susceptible to inhibition by an IGF-1 kinase inhibitor. For example, the kit can comprise a labeled compound or agent capable of detecting multiple 4-gene index proteins or nucleic acids (or AFP protein or nucleic acid) in a biological sample, or primers for use in PCR amplification, and means for determining the amounts of the proteins or mRNAs in the sample (e.g., antibodies which binds the proteins or a fragment thereof, or oligonucleotide probes which binds to the mRNAs, or derived cDNAs). Kits can also include standards or reference samples, and instructions for interpreting the results obtained using the kit.

For oligonucleotide-based kits, the kit can comprise, for example (e.g. for each 4-gene index gene, or AFP): (1) an oligonucleotide, e.g., a detectably labeled oligonucleotide, which hybridizes to a nucleic acid sequence encoding a 4-gene index gene or (2) a pair of primers useful for amplifying a 4-gene index nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit. The kit may also comprise a DNA microarray chip with oligonucleotide probes specific for each of the genes (e.g. each of the genes of the 4-gene index; or AFP).

The present invention further provides any of the methods described herein for treating tumors or tumor metastases, or cancer, in a patient comprising administering to the patient a therapeutically effective amount of an IGF-1R kinase inhibitor, with in addition, simultaneously or sequentially, one or more other cytotoxic, chemotherapeutic or anti-cancer agents, or compounds that enhance the effects of such agents. In the context of this invention, other anti-cancer agents includes, for example, other cytotoxic, chemotherapeutic or anti-cancer agents, or compounds that enhance the effects of such agents, anti-hormonal agents, angiogenesis inhibitors, agents that inhibit or reverse EMT (e.g. TGF-beta receptor inhibitors), tumor cell pro-apoptotic or apoptosis-stimulating agents, histone deacetylase (HDAC) inhibitors, histone demethylase inhibitors, DNA methyltransferase inhibitors, signal transduction inhibitors, anti-proliferative agents, anti-HER2 antibody or an immunotherapeutically active fragment thereof, anti-proliferative agents, COX II (cyclooxygenase II) inhibitors, and agents capable of enhancing antitumor immune responses, as described herein.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6 MP), 6-thiocguanine (6TG), cytarabine (Ara-C), 5-fluorouracil (5-FU), capecitabine (e.g.XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: amifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, exemestane, anastrozole, letrozole, vorozole, formestane, fadrozole, aminoglutethimide, testolactone, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N-6-(3-pyridinylcarbonyl)-D-lysyl-L-leucyl-N-6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4,20-nitro-3-(trifluoromethyl)phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides any of the methods described herein for treating tumors or tumor metastases in a patient comprising administering to the patient a therapeutically effective amount of an IGF-1R kinase inhibitor, and in addition, simultaneously or sequentially, one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); sunitinib (Pfizer); angiozyme, a synthetic ribozyme from Ribozyme (Boulder, Colo.) and Chiron (Emeryville, Calif.); and antibodies to VEGF, such as bevacizumab (e.g. AVASTINT™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more tumor cell pro-apoptotic or apoptosis-stimulating agents.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more signal transduction inhibitors.

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); EGFR kinase inhibitors (see herein below); Met kinase inhibitors (e.g. PF-2341066); ras inhibitors; raf inhibitors; MEK inhibitors; mTOR inhibitors, including mTOR inhibitors that bind to and directly inhibits both mTORC1 and mTORC2 kinases (e.g. OSI-027 (4-(4-amino-5-(7-methoxy-1H-indol-2-yl)imidazo[5,1-f][1,2,4]triazin-7-yl)cyclohexanecarboxylic acid hydrochloride), OSI Pharmaceuticals, Inc.); mTOR inhibitors that are dual PI3K/mTOR kinase inhibitors, such as for example the compound PI-103 as described in Fan, Q-W et al (2006) Cancer Cell 9:341-349 and Knight, Z. A. et al. (2006) Cell 125:733-747; mTOR inhibitors that are dual inhibitors of mTOR kinase and one or more other PIKK (or PIK-related) kinase family members. Such members include MEC1, TEL1, RAD3, MEI-41, DNA-PK, ATM, ATR, TRRAP, PI3K, and PI4K kinases; cyclin dependent kinase inhibitors; protein kinase C inhibitors; PI-3 kinase inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

EGFR kinase inhibitors include, for example: [6,7-bis(2-methoxyethoxy)-4-quinazolin-4-yl]-(3-ethynylphenyl) amine (also known as OSI-774, erlotinib, or TARCEVA™ (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); gefitinib (also known as ZD1839 or IRESSA™; Astrazeneca) (Woodburn et al., 1997, Proc. Am. Assoc. Cancer Res. 38:633); and antibody-based EGFR kinase inhibitors. A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, TARCEVA™), or other salt forms (e.g. erlotinib mesylate). Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX-447 (Medarex/Merck KgaA).

EGFR kinase inhibitors also include, for example multi-kinase inhibitors that have activity on EGFR kinase, i.e. inhibitors that inhibit EGFR kinase and one or more additional kinases. Examples of such compounds include the EGFR and HER2 inhibitor CI-1033 (formerly known as PD183805; Pfizer); the EGFR and HER2 inhibitor GW-2016 (also known as GW-572016 or lapatinib ditosylate; GSK); the EGFR and JAK 2/3 inhibitor AG490 (a tyrphostin); the EGFR and HER2 inhibitor ARRY-334543 (Array BioPharma); BIBW-2992, an irreversible dual EGFR/HER2 kinase inhibitor (Boehringer Ingelheim Corp.); the EGFR and HER2 inhibitor EKB-569 (Wyeth); the VEGF-R2 and EGFR inhibitor ZD6474 (also known as ZACTIMAT™; AstraZeneca Pharmaceuticals), and the EGFR and HER2 inhibitor BMS-599626 (Bristol-Myers Squibb).

ErbB2 receptor inhibitors include, for example: ErbB2 receptor inhibitors, such as lapatinib or GW-282974 (both GlaxoSmithKline), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, an anti-HER2 antibody (e.g. trastuzumab (Genentech)), or an immunotherapeutically active fragment thereof.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, one or more additional anti-proliferative agents.

Additional antiproliferative agents include, for example. Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217, and FGFR kinase inhibitors.

Examples of PDGFR kinase inhibitors that can be used according to the present invention include Imatinib (GLEEVEC®; Novartis); SU-12248 (sunitinib malate, SUTENT®; Pfizer); Dasatinib (SPRYCEL®; BMS; also known as BMS-354825); Sorafenib (NEXAVAR®; Bayer; also known as Bay-43-9006); AG-13736 (Axitinib; Pfizer); RPR127963 (Sanofi-Aventis); CP-868596 (Pfizer/OSI Pharmaceuticals); MLN-518 (tandutinib; Millennium Pharmaceuticals); AMG-706 (Motesanib; Amgen); ARAVA® (leflunomide; Sanofi-Aventis; also known as SU101), and OSI-930 (OSI Pharmaceuticals); Additional preferred examples of low molecular weight PDGFR kinase inhibitors that are also FGFR kinase inhibitors that can be used according to the present invention include XL-999 (Exelixis); SU6668 (Pfizer); CHIR-258/TKI-258 (Chiron); RO4383596 (Hoffmann-La Roche) and BIBF-1120 (Boehringer Ingelheim).

Examples of FGFR kinase inhibitors that can be used according to the present invention include RO-4396686 (Hoffmann-La Roche); CHIR-258 (Chiron; also known as TKI-258); PD 173074 (Pfizer); PD 166866 (Pfizer); ENK-834 and ENK-835 (both Enkam Pharmaceuticals A/S); and SU5402 (Pfizer). Additional preferred examples of low molecular weight FGFR kinase inhibitors that are also PDGFR kinase inhibitors that can be used according to the present invention include XL-999 (Exelixis); SU6668 (Pfizer); CHIR-258/TKI-258 (Chiron); RO4383596 (Hoffmann-La Roche), and BIBF-1120 (Boehringer Ingelheim).

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially,

a COX II (cyclooxygenase II) inhibitor. Examples of useful COX-II inhibitors include alecoxib (e.g. CELEBREX™), valdecoxib, and rofecoxib.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the IGF-1R kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a Ito 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides any of the methods described herein for treating cancer, or tumors or tumor metastases, in a patient comprising administering to the patient a therapeutically effective amount of an IGF-1R kinase inhibitor, and in addition, simultaneously or sequentially, treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4, ipilimumab (a.k.a. MDX-010, Bristol-Myers Squibb/Medarex), and other agents capable of blocking CTLA4. Specific CTLA4 antibodies that can be used in the present invention include those described in U.S. Pat. No. 6,682,736.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

As used herein, the term “patient” preferably refers to a human in need of treatment with an IGF-1R kinase inhibitor for cancer, including refractory versions of such cancers that have failed to respond to other treatments. For methods involving AFP the cancer is HCC. For other methods the cancer may be HCC, as described in the methods herein, but such methods are also anticipated to work where instead of HCC, the cancer, or tumors and tumor metastases, are NSCL (non-small cell lung), pancreatic, head and neck, oral or nasal squamous cell carcinoma, colon, ovarian or breast cancers, lung cancer, bronchioloalveolar cell lung cancer, bone cancer, skin cancer, cancer of the head or neck, HNSCC, cutaneous or intraocular melanoma, uterine cancer, hepatocellular carcinoma, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, colorectal cancer, cancer of the endocrine system, cancer of the thyroid gland, cancer of the parathyroid gland, cancer of the adrenal gland, adrenocortical carcinoma (ACC), sarcoma of soft tissue, Ewing's sarcoma, rhabdomyosarcoma, myeloma, multiple myeloma, cancer of the urethra, cancer of the penis, prostate cancer, cancer of the bladder, cancer of the ureter, carcinoma of the renal pelvis, mesothelioma, biliary cancer, cancer of the kidney, renal cell carcinoma, chronic or acute leukemia, lymphocytic lymphomas, neuroblastoma, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Thus, this invention provides any of the methods described herein that involve HCC and not AFP, but wherein HCC is replaced by any of the above cancers. In addition to cancer, such methods may also be used for precancerous conditions or lesions, including, for example, oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, liver cirrhosis or scarring, and precancerous cervical conditions.

Thus, for example, the present invention further provides a method of identifying a tumor as likely to be responsive or non-responsive to treatment with an IGF-1R kinase inhibitor, comprising: measuring in the tumor cells the relative expression level of each gene of a 4-gene signature (4GS), wherein the 4GS consists essentially of the following genes: INSR, IGF2, IGFBP3 and IGFBP7; calculating a 4GS index score for said tumor cells according to the equation:

$$4 GS index score = \frac{1}{n} {\sum }_{i ∈ IGF} {g}_{i} \cdot r ,$$

wherein: IGF=the genes IGF2, INSR, IGFBP3, and IGFBP7; n=number of genes in the gene signature=4; gᵢ=median centered expression value of gene i; and r=+1 for IGF2 and INSR, and r=−1 for IGFBP3 and IGFBP7; and determining if the 4GS index score is above a defined threshold that indicates that the tumor is likely to be responsive to an IGF-1R kinase inhibitor, or below said threshold and thus likely to be non-responsive to an IGF-1R kinase inhibitor. The tumor may be any of those listed herein.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease). As used herein the term can apply to any of the treatments or agents described herein, when used as single agents or combinations.

For purposes of the present invention, “co-administration of” and “co-administering” an IGF-1R kinase inhibitor with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The additional agent can be administered prior to, at the same time as, or subsequent to administration of the IGF-1R kinase inhibitor, or in some combination thereof. Where the IGF-1R kinase inhibitor is administered to the patient at repeated intervals, e.g., during a standard course of treatment, the additional agent can be administered prior to, at the same time as, or subsequent to, each administration of the IGF-1R kinase inhibitor, or some combination thereof, or at different intervals in relation to the IGF-1R kinase inhibitor treatment, or in a single dose prior to, at any time during, or subsequent to the course of treatment with the IGF-1R kinase inhibitor.

The IGF-1R kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the IGF-1R kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of IGF-1R kinase inhibitor being used (for example, small molecule, antibody, RNAi, ribozyme or antisense construct), and the medical judgement of the prescribing physician as based, e.g., on the results of published clinical studies.

The amount of IGF-1R kinase inhibitor administered and the timing of IGF-1R kinase inhibitor administration will depend on the type (species, gender, age, weight, etc.) and condition of the patient being treated, the severity of the disease or condition being treated, and on the route of administration. For example, small molecule IGF-1R kinase inhibitors can be administered to a patient in doses ranging from 0.001 to 100 mg/kg of body weight per day or per week in single or divided doses, or by continuous infusion (see for example, International Patent Publication No. WO 01/34574). In particular, compounds such as OSI-906, or similar compounds, can be administered to a patient in doses ranging from 5-300 mg per day (e.g. 150 mg BID (2 times a day)), or 100-1600 mg per week, in single or divided doses, or by continuous infusion. Antibody-based IGF-1R kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The IGF-1R kinase inhibitors and other additional agents can be administered either separately or together by the same or different routes, and in a wide variety of different dosage forms. For example, the IGF-1R kinase inhibitor is preferably administered orally or parenterally. Where the IGF-1R kinase inhibitor is OSI-906, or a similar such compound, oral administration is preferable. Both the IGF-1R kinase inhibitor and other additional agents can be administered in single or multiple doses.

The IGF-1R kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The IGF-1R kinase inhibitor can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous IGF-1R kinase inhibitors should be selected so as to avoid denaturation and/or degradation and loss of biological activity of the inhibitor.

Methods of preparing pharmaceutical compositions comprising an IGF-1R kinase inhibitor are known in the art, and are described, e.g. in International Patent Publication No. WO 01/34574. In view of the teaching of the present invention, methods of preparing pharmaceutical compositions comprising an IGF-1R kinase inhibitor will be apparent from the above-cited publications and from other known references, such as Remington's Pharmaceutical Sciences, Mack Publishing Company, Easton, Pa., 18^{th} edition (1990).

For oral administration of IGF-1R kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the IGF-1R kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous IGF-1R kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising an IGF-1R kinase inhibitor in about 0.1% (w/v) to about 5% (w/v) concentration can be prepared.

As used herein, the term “IGF-1R kinase inhibitor” refers to any IGF-1R kinase inhibitor that is currently known in the art, and includes any chemical entity that, upon administration to a patient, results in inhibition of a biological activity specifically associated with activation of the IGF-1 receptor (e.g. in humans, the protein encoded by GenelD: 3480) in the patient, and resulting from the binding to IGF-1R of its natural ligand(s). Such IGF-1R kinase inhibitors include any agent that can block IGF-1R activation and the downstream biological effects of IGF-1R activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors include, for example imidazopyrazine IGF-1R kinase inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Additional examples of IGF-1R kinase inhibitors include those in International Patent Publication No. WO 05/097800, that describes 6,6-bicyclic ring substituted heterobicyclic protein kinase inhibitors, International Patent Publication No. WO 05/037836, that describes imidazopyrazine IGF-1R kinase inhibitors, International Patent Publication Nos. WO 03/018021 and WO 03/018022, that describe pyrimidines for treating IGF-1R related disorders, International Patent Publication Nos. WO 02/102804 and WO 02/102805, that describe cyclolignans and cyclolignans as IGF-1R inhibitors, International Patent Publication No. WO 02/092599, that describes pyrrolopyrimidines for the treatment of a disease which responds to an inhibition of the IGF-1R tyrosine kinase, International Patent Publication No. WO 01/72751, that describes pyrrolopyrimidines as tyrosine kinase inhibitors, and in International Patent Publication No. WO 00/71129, that describes pyrrolotriazine inhibitors of kinases, and in International Patent Publication No. WO 97/28161, that describes pyrrolo[2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), International Patent Publication No. WO 00/35455, that describes heteroaryl-aryl ureas as IGF-1R inhibitors, International Patent Publication No. WO 03/048133, that describes pyrimidine derivatives as modulators of IGF-1R, International Patent Publication No. WO 03/024967, WO 03/035614, WO 03/035615, WO 03/035616, and WO 03/035619, that describe chemical compounds with inhibitory effects towards kinase proteins, International Patent Publication No. WO 03/068265, that describes methods and compositions for treating hyperproliferative conditions, International Patent Publication No. WO 00/17203, that describes pyrrolopyrimidines as protein kinase inhibitors, Japanese Patent Publication No. JP 07/133,280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

IGF-1R kinase inhibitors particularly useful in this invention include compounds represented by Formula (I) (see below), as described in US Published Patent Application US 2006/0235031, where their preparation is described in detail. PQIP (cis-3-[3-(4-Methyl-piperazin-1-yl)-cyclobutyl]1-(2-phenyl-quinolin-7-yl)-imidazo[1,5-a]pyrazin-8-ylamine) and OSI-906 (cis-3-[8-amino-1-(2-phenyl-quinolin-7-yl)-imidazo[1,5-a]pyrazin-3-yl]-1-methyl-cyclobutanol) represents IGF-1R kinase inhibitors according to Formula (I).

OSI-906 has the structure as follows:

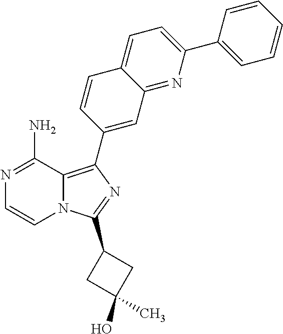

PQIP has the structure as follows:

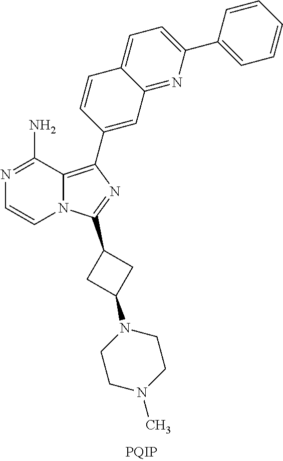

An IGF-1R kinase inhibitor of Formula (I), as described in US Published Patent Application

US 2006/0235031, is represented by the formula:

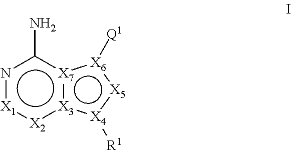

or a pharmaceutically acceptable salt thereof, wherein:

X₁, and X₂ are each independently N or C−(E¹)ₐₐ;

X₅ is N, C−(E¹)ₐₐ, or N−(E¹)ₐₐ;

X₃, X₄, X₆, and X₇ are each independently N or C;

wherein at least one of X₃, X₄, X₅, X₆, and X₇ is independently N or N−(E¹)ₐₐ;

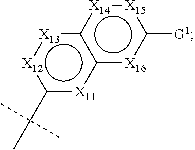

X₁₁, X₁₂, X₁₃, X₁₄, X₁₅, and X₁₆ are each independently N, C-(E¹¹)_{bb}, or N⁺—O⁻;
wherein at least one of X₁₁, X₁₂, X₁₃, X₁₄, X₁₅, and X₁₆ is N or N⁺—O⁻;

R¹ is absent, C₀₋₁₀alkyl, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G^{1l} substituents;

E¹, E^{1l}, G¹ and G⁴¹ are each independently halo, —CF₃, —OCF₃, —OR², —NR²R³(R^{2a})ⱼ₁, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —C(═O)SR², —NR²C(═NR³)NR^{2a}R³a, —NR²C(═NR³)OR^{2a}, —NR²C(═NR³)SR^{2a}, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkenylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃—OR²²², —NR²²²R³³³(R^{222a})ⱼ₁ₐ, —C(═O)R²²², —CO₂R²²², —C(═O)NR²²²R³³³, —NO₂, —CN, S(═O)ⱼ₁ₐR²²², —SO₂NR²²²R³³³, —NR²²²C(═O)R³³³, —NR²²²C(═O)OR³³³, —NR²²²C(═O)NR³³³R^{222a}, —NR²²²S (O)ⱼ₁ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;

or E¹, E¹¹, or G¹ optionally is —(W¹)ₙ—(Y¹)ₘ—R⁴;

or E¹, E¹¹, G¹, or G⁴¹ optionally independently is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²², —NR²²²R³³³(R^{222a})ⱼ₂ₐ, —C(O)R²²², —CO₂R²²², C(═O)NR²²²R³³³, —NO₂, —CN, —S(O)ⱼ₂ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₂ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²″, or —SC(═O)NR²²²R³³³ substituents;

G¹¹ is halo, oxo, —CF₃, —OCF₃, OR²¹, —NR²¹R³¹(R^{2a1})ⱼ₄, —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, —NR²¹C(═NR³¹)NR^{2a1}R^{3a1}, —NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₁₋₁₀alkylidene, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈ alkyl, cycloC₃₋₈alkenyl, cycloC₃₋₈ alkylC₁₋₁₀alkyl, cycloC₃₋₈ alkenylC₁₋₁₀alkyl, cycloC₃₋₈ alkylC₂₋₁₀alkenyl, cycloC₃₋₈ alkenylC₂₋₁₀alkenyl, cycloC₃₋₈ alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²²¹, —NR²²²¹R³³³¹(R^{222a1})ⱼ₄ₐ, —C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹C(═O)R³³³¹R^{222a1}, —NRH²²²¹S(O)ⱼ₄ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is aryl C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²²¹, —NR²²²¹R³³³¹(R^{222a1})ⱼ₅ₐ, —C(O)R²²²¹, —CO₂R²²²¹, —C(═O)NR²²²¹R³³³¹, —NO₂, —CN, —S(O)ⱼ₅ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₅ₐR³³³¹, —C(═S)OR²²²¹, —C(═O)SR²²²¹, NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is C, taken together with the carbon to which it is attached forms a C═C double bond which is substituted with R⁵ and G¹¹¹;

R², R^{2a}, R³, R^{3a}, R²²², R^{222a}, R³³³, R^{333a}, R²¹, R^{2a1}, R³¹, R^{31a}, R²²²¹, R^{222a1}, R³³³¹, and R^{333a1} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈ alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹¹ substituents;

or in the case of —NR²R³(R^{2a})ⱼ₁ or —NR²²²R³³³(R^{222a})ⱼ₁ₐ or —NR²²²R³³³(R^{222a})ⱼ₂ₐ or —NR²¹R³¹(R^{2a1})ⱼ₄ or —NR²²²¹R³³³¹(R^{222a1})ⱼ₄ₐ or —NR²²²¹R³³³¹(R^{222a1})ⱼ₅ₐ, then R² and R³, or R²²² and R³³³, or R²²²¹ and R³³³¹, respectfully, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²²¹ and R³³³¹ are attached;

W¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C≡C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂— —OC(O)N(R⁷)—, —N(R⁷)C(O)N(R⁸)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁸)—, —N(R⁷)S(O)₂N(R⁸)—, —C(O)N(R⁷)C(O)—, —S(O)N(R⁷)C(O)—, —S(O)₂N(R⁷)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁸)—, —N(R⁷)SO₂N(R⁸)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁸)—, —CH(R⁷)N(C(O)R⁸)—, —CH(R⁷)N(SO₂R⁸)—, —CH(R⁷)O—, —CH(R⁷)S—, —CH(R⁷)N(R⁸)—, —CH(R⁷)N(C(O)R⁸)—, —CH(R⁷)N(C(O)OR⁸)—, —CH(R⁷)N(SO₂R⁸)—, —CH(R⁷)C(═NOR⁸)—, —CH(R⁷)C(O)—, —CH(R⁷)CH(OR⁸)—, —CH(R⁷)C(O)N(R⁸)—, —CH(R⁷)N(R⁸)C(O)—, —CH(R⁷)N(R⁸)S(O)—, —CH(R⁷)N(R⁸)S(O)₂—, —CH(R⁷)OC(O)N(R⁸)—, —CH(R⁷)N(R⁸)C(O)N(R^{7a})—, —CH(R⁷)NR⁸C(O)O—, —CH(R⁷)S(O)N(R⁸)—, —CH(R⁷)S(O)₂N(R⁸)—, —CH(R⁷)N(C(O)R⁸)S(O)—, —CH(R⁷)N(C(O)R⁸)S(O)—, —CH(R⁷)N(R⁸)S(O)N(R^{7a})—, —CH(R⁷)N(R⁸)S(O)₂N(R^{7a})—, —CH(R⁷)C(O)N(R⁸)C(O)—, —CH(R⁷)S(O)N(R⁸)C(O)—, —CH(R⁷)S(O)₂N(R⁸)C(O)—, —CH(R⁷)OS(O)N(R⁸)—, —CH(R⁷)OS(O)₂N(R⁸)—, —CH(R⁷)N(R⁸)S(O)O—, —CH(R⁷)N(R⁸)S(O)₂O—, —CH(R⁷)N(R⁸)S(O)C(O)—, —CH(R⁷)N(R⁸)S(O)₂C(O)—, —CH(R⁷)SON(C(O)R⁸)—, —CH(R⁷)SO₂N(C(O)R⁸)—, —CH(R⁷)N(R⁸)SON(R^{7a})—, —CH(R⁷)N(R⁸)SO₂N(R^{7a})—, —CH(R⁷)C(O)O—, —CH(R⁷)N(R⁸)P(OR^{7a})O—, —CH(R⁷)N(R⁸)P(OR^{7a})—, —CH(R⁷)N(R⁸)P(O)(OR^{7a})O—, —CH(R⁷)N(R⁸)P(O)(OR^{7a})—, —CH(R⁷)N(C(O)R⁸)P(OR^{7a})O—, —CH(R⁷)N(C(O)R⁸)P(OR^{7a})—, —CH(R⁷)N(C(O)R⁸)P(O)(OR^{7a})O—, or —CH(R⁷)N(C(O)R⁸)P(OR^{7a})—;

R⁵, R⁶, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₂₋₁₀alkynyl, cycloC₃₋₈alkyl, cycloC₃₋₈ alkenyl, cycloC₃₋₈alkylC₁₋₁₀-alkyl, cycloC₃₋₈alkenylC₁₋₁₀alkyl, cycloC₃₋₈alkylC₂₋₁₀alkenyl, cycloC₃₋₈ alkenylC₂₋₁₀alkenyl, cycloC₃₋₈alkylC₂₋₁₀alkynyl, cycloC₃₋₈alkenyl C₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, —NR⁷⁷C(═O)OR⁸⁷, —NR⁷⁷C(═O)NR⁷⁸R⁸⁷, —NR⁷⁷S(O)ⱼ₅ₐR⁸⁷, —C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷⁸, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;

or R⁵ with R⁶ are optionally taken together with the carbon atom to which they are attached to form a 3-10 membered saturated or unsaturated ring, wherein said ring is optionally substituted with one or more independent R⁶⁹ substituents and wherein said ring optionally includes one or more heteroatoms;

R⁷, R^{7a}, and R⁸ are each independently acyl, C₀₋₁₀alkyl, C₂₋₁₀alkenyl, aryl, heteroaryl, heterocyclyl or cycloC₃₋₁₀alkyl, any of which is optionally substituted by one or more independent G111 substituents;

R⁴ is C₀₋₁₀alkyl, C₂₋₁₀alkenyl, C₂₋₁₀alkynyl, aryl, heteroaryl, cycloC₃₋₁₀alkyl, heterocyclyl, cycloC₃₋₈alkenyl, or heterocycloalkenyl, any of which is optionally substituted by one or more independent G⁴¹ substituents;

R⁶⁹ is halo, —OR⁷⁸, —SH, —NR⁷⁸R⁸⁸, —COR⁷⁸—C(═O)NR^{78e}, —NO₂, —CN, —S(O)ⱼ₈R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₂₋₁₀alkenyl, C₁₋₁₀alkylthioC₂₋₁₀oalkynyl, cycloC₃₋₈alkyl, cycloC₃₋₈alkenyl, cycloC₃₋₈ alkylC₁₋₁₀alkyl, cycloC₃₋₈ alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₁₀alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents;

or R⁶⁹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —C(═O)NR⁷⁷⁸R⁸⁸⁸, —SO₂NR⁷⁷⁸R⁸⁸⁸, or —NR⁷⁷⁸R⁸⁸⁸ substituents;

or in the case of —NR⁷⁸R⁸⁸, R⁷⁸ and R⁸⁸ are optionally taken together with the nitrogen atom to which they are attached to form a 3-10 membered saturated or unsaturated ring, wherein said ring is optionally substituted with one or more independent halo, cyano, hydroxy, nitro, C₁₋₁₀alkoxy, —SO₂NR⁷⁷⁸R⁸⁸⁸, or —NR⁷⁷⁸R⁸⁸⁸ substituents, and wherein said ring optionally includes one or more heteroatoms other than the nitrogen to which R⁷⁸ and R⁸⁸ are attached;

R⁷⁷, R⁷⁸, R⁸⁷, R⁸⁸, R⁷⁷⁸, and R⁸⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈ alkyl, cycloC₃₋₈alkenyl, cycloC₃₋₈ alkylC₁₋₁₀alkyl, cycloC₃₋₈ alkenylC₁₋₁₀alkyl, cycloC₃₋₈ alkylC₂₋₁₀alkenyl, cycloC₃₋₈ alkenylC₂₋₁₀alkenyl, cycloC₃₋₈ alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;

or R⁷⁷, R⁷⁸, R⁸⁷, R⁸⁸, R⁷⁷⁸, and R⁸⁸⁸ are each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;

n, m, j1, j1a, j2a, j4, j4a, j5a, j7, and j8 are each independently 0, 1, or 2; and aa and bb are each independently 0 or 1.

Additional, specific examples of IGF-1R kinase inhibitors that can be used according to the present invention include h7C10 (Centre de Recherche Pierre Fabre), an IGF-1 antagonist; EM-164 (ImmunoGen Inc.), an IGF-1R modulator; CP-751871 (Pfizer Inc.), an IGF-1 antagonist; lanreotide (Ipsen), an IGF-1 antagonist; IGF-1R oligonucleotides (Lynx Therapeutics Inc.); IGF-1 oligonucleotides (National Cancer Institute); IGF-1R protein-tyrosine kinase inhibitors in development by Novartis (e.g. NVP-AEW541, Garcia-Echeverria, C. et al. (2004) Cancer Cell 5:231-239; or NVP-ADW742, Mitsiades, C. S. et al. (2004) Cancer Cell 5:221-230); IGF-1R protein-tyrosine kinase inhibitors (Ontogen Corp); OSI-906 (OSI Pharmaceuticals); AG-1024 (Camirand, A. et al. (2005) Breast Cancer Research 7:R570-R579 (DOI 10.1186/bcr1028); Camirand, A. and Pollak, M. (2004) Brit. J. Cancer 90:1825-1829; Pfizer Inc.), an IGF-1 antagonist; the tyrphostins AG-538 and I-OMe-AG 538; BMS-536924, a small molecule inhibitor of IGF-1R; PNU-145156E (Pharmacia & Upjohn SpA), an IGF-1 antagonist; BMS 536924, a dual IGF-1R and IR kinase inhibitor (Bristol-Myers Squibb; Huang, F. et al. (2009) Cancer Res. 69(1):161-170); BMS-554417, a dual IGF-1R and IR kinase inhibitor (Bristol-Myers Squibb; Haluska P, et al. Cancer Res 2006; 66(1):362-71); EW541 (Novartis); GSK621659A (Glaxo Smith-Kline); INSM-18 (Insmed); and XL-228 (Exelixis).

Antibody-based IGF-1R kinase inhibitors include any anti-IGF-1R antibody or antibody fragment that can partially or completely block IGF-1R activation by its natural ligand. Antibody-based IGF-1R kinase inhibitors also include any anti-IGF-1 antibody or antibody fragment that can partially or completely block IGF-1R activation. Non-limiting examples of antibody-based IGF-1R kinase inhibitors include those described in Larsson, O. et al (2005) Brit. J. Cancer 92:2097-2101 and Ibrahim, Y. H. and Yee, D. (2005) Clin. Cancer Res. 11:944s-950s, or being developed by Imclone (e.g. A12) or Schering-Plough Research Institute (e.g. 19D12; or as described in US Patent Application Publication Nos. US 2005/0136063 A1 and US 2004/0018191 A1). The IGF-1R kinase inhibitor can be a monoclonal antibody, or an antibody or antibody fragment having the binding specificity thereof.

Additional antibody-based IGF-1R kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against IGF-1R can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al, 1985, Monoclonal Antibodies and Cancer Therapy, Alan R. Liss, Inc., pp. 77-96) Chimeric, humanized or human monoclonal antibodies may be used (Hudson, P. J. and Souriau, C. (2003) Nature Medicine, 9:129-134).

Alternatively, techniques described for the production of single chain antibodies (see, e.g., U.S. Pat. No. 4,946,778) can be adapted to produce anti-IGF-1R single chain antibodies. Antibody-based IGF-1R kinase inhibitors useful in practicing the present invention also include anti-IGF-1R antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to IGF-1R.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-IGF-1R antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

IGF-1R kinase inhibitors for use in the present invention can alternatively be based on antisense oligonucleotide constructs. Anti-sense oligonucleotides, including anti-sense RNA molecules and anti-sense DNA molecules, would act to directly block the translation of IGF-1R mRNA by binding thereto and thus preventing protein translation or increasing mRNA degradation, thus decreasing the level of IGF-1R kinase protein, and thus activity, in a cell. For example, antisense oligonucleotides of at least about 15 bases and complementary to unique regions of the mRNA transcript sequence encoding IGF-1R can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as IGF-1R kinase inhibitors for use in the present invention. IGF-1R gene expression can be reduced by contacting the tumor, subject or cell with a small double stranded RNA (dsRNA), or a vector or construct causing the production of a small double stranded RNA, such that expression of IGF-1R is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as IGF-1R kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of IGF-1R mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as IGF-1R kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

In the context of the methods of treatment of this invention, IGF-1R kinase inhibitors are used as a composition comprised of a pharmaceutically acceptable carrier and a non-toxic therapeutically effective amount of an IGF-1R kinase inhibitor compound (including pharmaceutically acceptable salts thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions used in the present invention comprising an IGF-1R kinase inhibitor compound (including pharmaceutically acceptable salts thereof) as active ingredient, can include a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the IGF-1R kinase inhibitor compounds (including pharmaceutically acceptable salts thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an IGF-1R kinase inhibitor compound (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

An IGF-1R kinase inhibitor compound (including pharmaceutically acceptable salts thereof) used in this invention, can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, the pharmaceutical composition can comprise an IGF-1R kinase inhibitor compound in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition used fot this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions for the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an IGF-1R kinase inhibitor compound (including pharmaceutically acceptable salts thereof), via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions for this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an IGF-1R kinase inhibitor compound (including pharmaceutically acceptable salts thereof) may also be prepared in powder or liquid concentrate form.

Dosage levels for the compounds used for practicing this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The present invention further provides for any of the “methods of treatment” described herein, a corresponding “method for manufacturing a medicament” for use with the same indications and under identical conditions or modalities described for the method of treatment, characterized in that an IGF-1R kinase inhibitor is used, such that where any additional agents, inhibitors or conditions are specified in alternative embodiments of the method of treatment they are also included in the corresponding alternative embodiment for the method for manufacturing a medicament. The present invention also provides an IGF-1R kinase inhibitor for use in any of the methods of treatment for cancer described herein.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons. (e.g. ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3^{rd} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Materials and Methods

IGF-1R/IR inhibitors: IGF-1R inhibitor compound OSI-906 was provided by OSI Pharmaceuticals, (Melville, N.Y.). OSIP-906 (cis-3-[8-amino-1-(2-phenyl-quinolin-7-yl)-imidazo[1,5-a]pyrazin-3-yl]-1-methyl-cyclobutanol) is synthesized by the methods described in patent application number WO 2005/097800. Compound identity and purity (>99%) were verified by ¹H and ¹³C nuclear magnetic resonance, mass spectrometry (MS), and high-performance liquid chromatography using Bruker Advance 400, WatersMicromass ZQ, and Waters LC Module I Plus instruments, respectively, as well as by elemental analysis. OSI-906 was dissolved in DMSO as a 10 mmol/L stock solution for use in biochemical or cellular assays in vitro.

Cell lines: Twenty-one HCC cell lines were purchased from either ATCC (HepG2, Hep3B, PLC/PRF/5, SK-Hepl, SNU-182, SNU-387, SNU-398, SNU-423, SNU-449, SNU-475) or Health Science Research Resources Bank (Japan) (HUH-1, HUH-6, HUH-7, HLE, HLF, JHH-1, JHH-2, JHH-4, JHH-5, JHH-6, JHH-7). All the cell lines were maintained in media as described by the vendors. For growth inhibition assays, cells were plated and allowed to proliferate for 24 hours. After 24 hours, cells had reached approximately 15% confluency, at which time serial dilutions of OSI-906 were added and the cells grown for a further 72 hours. Cell viability was assayed using the Cell Titer-Glo reagent (Promega Corp., Madison, Wis.).

Preparation of Protein Lysates and Western Blotting: Cells were rinsed with PBS and lysed in RIPA buffer (Sigma #R0278) containing protease and phosphatase inhibitor cocktails (Sigma #P2850, P8340, P5726). Cell lysates were cleared by centrifugation and subjected to western blotting. Antibodies included: E-cadherin (Santa Cruz #sc21791), ErbB3 (Santa Cruz #sc285), vimentin (BD Pharmingen #550513), Zebl (Santa Cruz #sc25388).

Analyses of EMT gene expression: cDNA was loaded on Custom TAQMAN® Array 384-Well Micro Fluidic Cards (Applied Biosystems) which were pre-loaded with primers for 19 EMT genes, and qPCR was run on 7900 HT Fast Real-Time PCR system (Applied Biosystems).

Taqman Assays: Total RNA was isolated with RNeasy kit (Qiagen) and treated with RNase-free DNase. Reverese transcription was performed with SuperScript III First-Strand Synthesis system (Invitrogen, Carlsbad, Calif.). The Gene Expression Assays were obtained from Applied Biosystems, Foster City, Calif. Quantitation of relative gene expression was conducted as described by the manufacturer using 50 ng of template. In order to determine relative expression across cell lines, amplification of AFP gene was compared to amplification of the gene for GAPDH as an internal standard.

Analyses of AFP expression in HCC tumors: The Affymetrix Human Genome U133 Plus 2.0 Array data in HCC from Chiang et al. [Chiang et al. 2008] were downloaded from GEO (accession number: GSE9829). CEL files were RMA (Robust Multichip Average) normalized using the Partek Genomics Suite v6.5 (Partek Inc., St. Louis, Mo., USA). Subsequently, the expression level of each gene was averaged over all probesets on the array.

TGFβ treatment: Cells were grown in medium supplemented with 10 ng/ml TGFβ (EMD Biosciences #616450) for 10 days with replating every 3-4 days. The cells were then lysed for either RT-PCR or CELLTITER-GLO® proliferation assays (Promega, Madison, Wis.).

Relative gene expression: Delta CT values were used to calculate relative gene expression values. The relative expression is calculated as the 2̂(−ΔCT) values from each cell line divided by the lowest 2̂(−ΔCT) value among 21 HCC cell lines.

Statistical analyses: Pearson correlation was analyzed for IGF axis 4-gene index score, AFP index scores, or E-cadherin index scores, and EC50 values generated from proliferation assays with OSI-906 treatment. The median-centered delta CT values from RT-PCR of AFP or E-cadherin were used as their index scores, and median-centered deltaCT values from RT-PCR of IGF-2, INSR, IGFBP3 and IGFBP7 were used to calculate IGF axis 4-gene index scores. To calculate IGF axis 4-gene index score (see equation below), each gene is assigned a sign (+1 for positive correlation with EC50 and −1 for negative correlation). The IGF axis 4-gene index scores are calculated as the average of median-centered deltaCT values adjusted with appropriate signs (times +1 for IGF-2 and IR, and times −1 for IGFBP3 and IGFBP7). The detailed steps are:
1. calculate the median delta CT value of each gene across all HCC cell lines.
2. calculate median-centered delta CT value: for each gene, delta CT value of each cell line minus median delta CT value from step #1 for that gene.
3. calculate 4-gene index: for each cell line, use values from #2 for each gene, 4-gene index score=(IGF-2+IR−IGFBP3−IGFBP7)/4.

IGF Axis 4-Gene Index Equation:

$$IGF axis 4 \text{-} gene index score = \frac{1}{n} {\sum }_{i ∈ IGF} {g}_{i} \cdot r$$

wherein IGF=genes in the IGF axis: IGF2, 1NSR, IGFBP3, and IGFBP7,

n=number of genes in the IGF axis=4, and

gi=median centered expression value of gene i.

r=+1 for IGF2 and INSR; r=−1 for IGFBP3 and IGFBP7.

Proliferation Assay. Proliferation was assayed using Cell Titer Glo assays (Promega) and was determined 72 hours following dosing with OSI-906. The basis of the assay is a luminescent quantitation of ATP present in a cell culture plate well. In essence, the greater the number of viable cells in the well, the greater the level of ATP present. The assay utilizes a substrate that binds ATP to produce a luminescent signal, which can be read on a luminometer. Unless otherwise noted, the manufacturer's instructions were followed exactly. Briefly, on Day 1, cells were plated in 120 μl of 10% serum-containing growth media at a density of 4000 cells/well in a white polystyrene 96 well assay plate. On day 2, cells were treated with 15 μl of 10× concentration of the IGF-1R inhibitor (e.g. OSI-906) or DMSO alone for a final well volume of 150 μl. After 72 h incubation with the inhibitor, the cells were assayed. Results were calculated as a fraction of the DMSO controlled cells.

Results and Discussion

TABLE 1

| | | % inhibition |
|---|---|---|
| Cell lines | EC50 (μM) | @ 5 μM |
| Hep G2 | 0.22 | 77% |
| Hep 3B | 0.35 | 67% |
| Huh-1 | 0.18 | 74% |
| Huh-6 | 0.25 | 41% |
| Huh-7 | 0.37 | 70% |
| JHH-5 | 0.19 | 80% |
| JHH-7 | 0.94 | 70% |
| PLC/PRF/5 | >10 | <40% |
| SK Hep-1 | >10 | <40% |
| SNU-182 | >10 | <40% |
| SNU-387 | >10 | <40% |
| SNU-398 | >10 | <40% |
| SNU-423 | >10 | <40% |
| SNU-449 | >10 | <40% |
| SNU-475 | >10 | <40% |
| HLE | >10 | <40% |
| HLF | >10 | <40% |
| JHH-1 | >10 | <40% |
| JHH-2 | >10 | <40% |
| JHH-4 | >10 | <40% |
| JHH-6 | >10 | <40% |

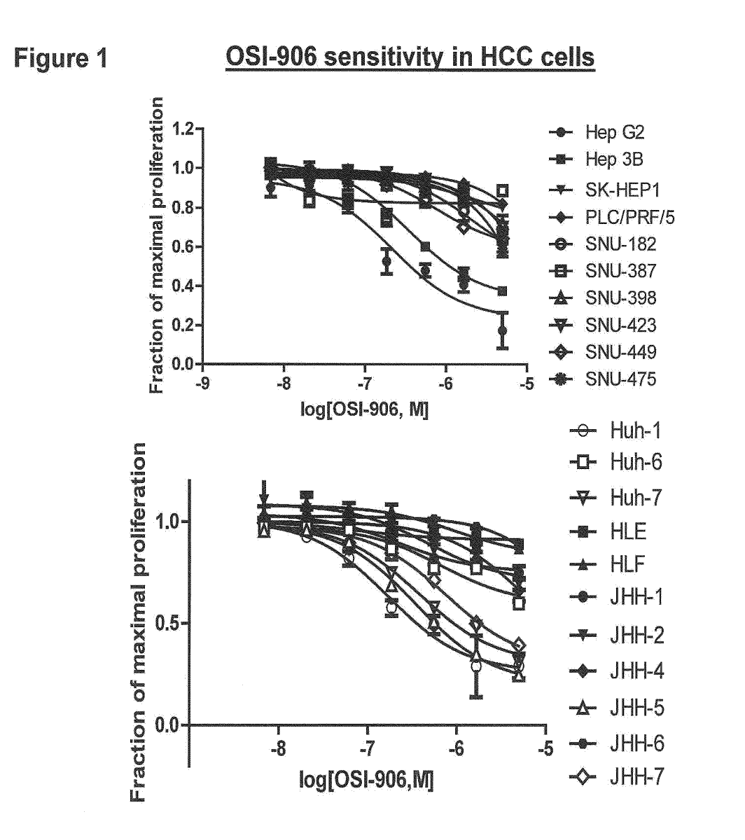

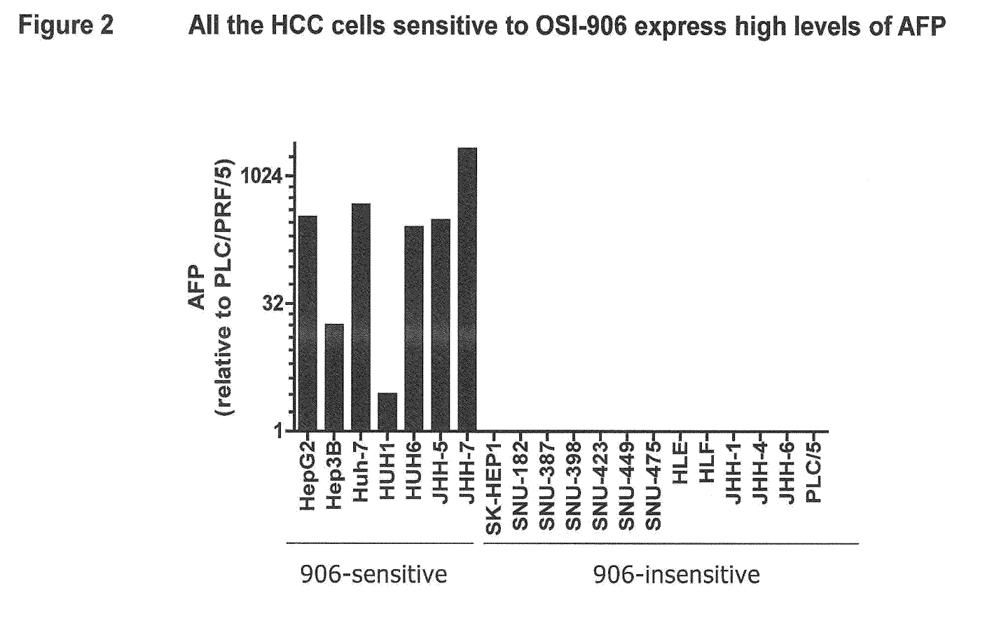

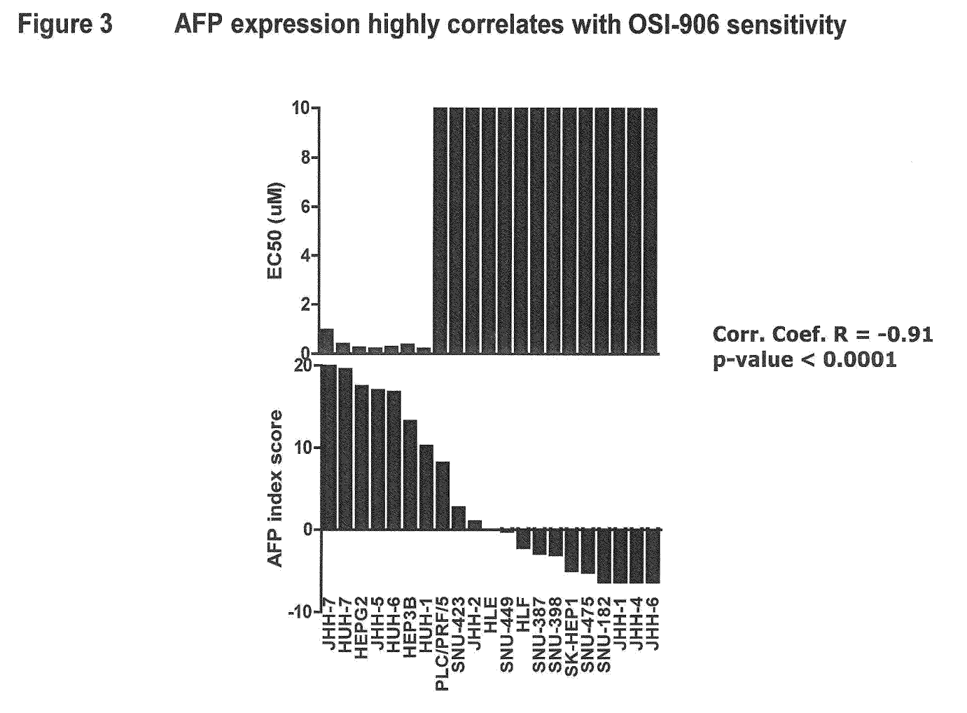

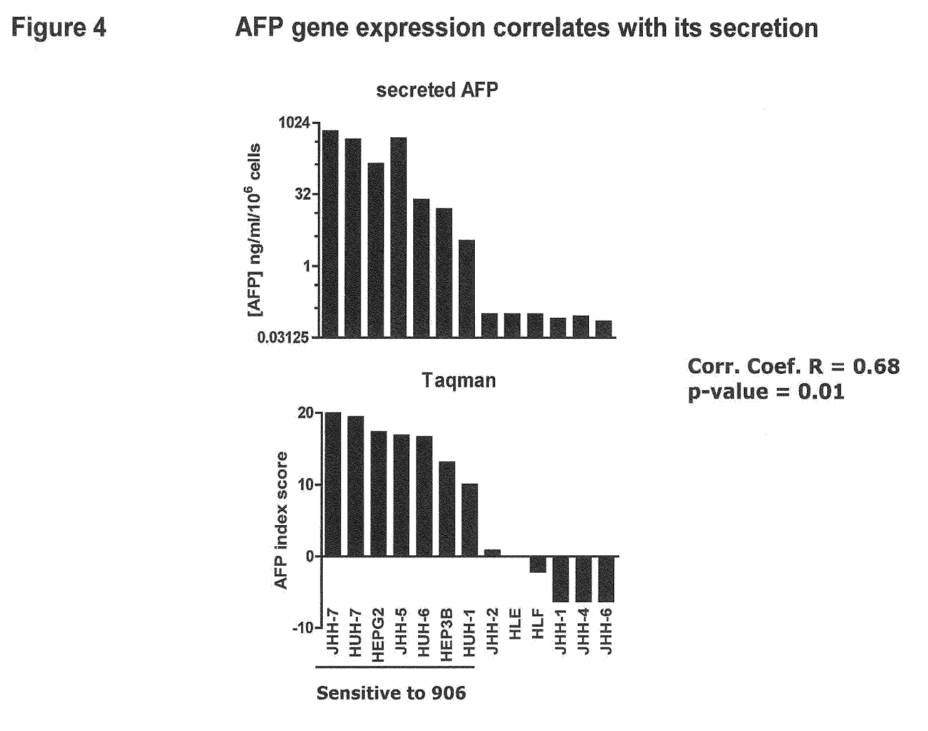

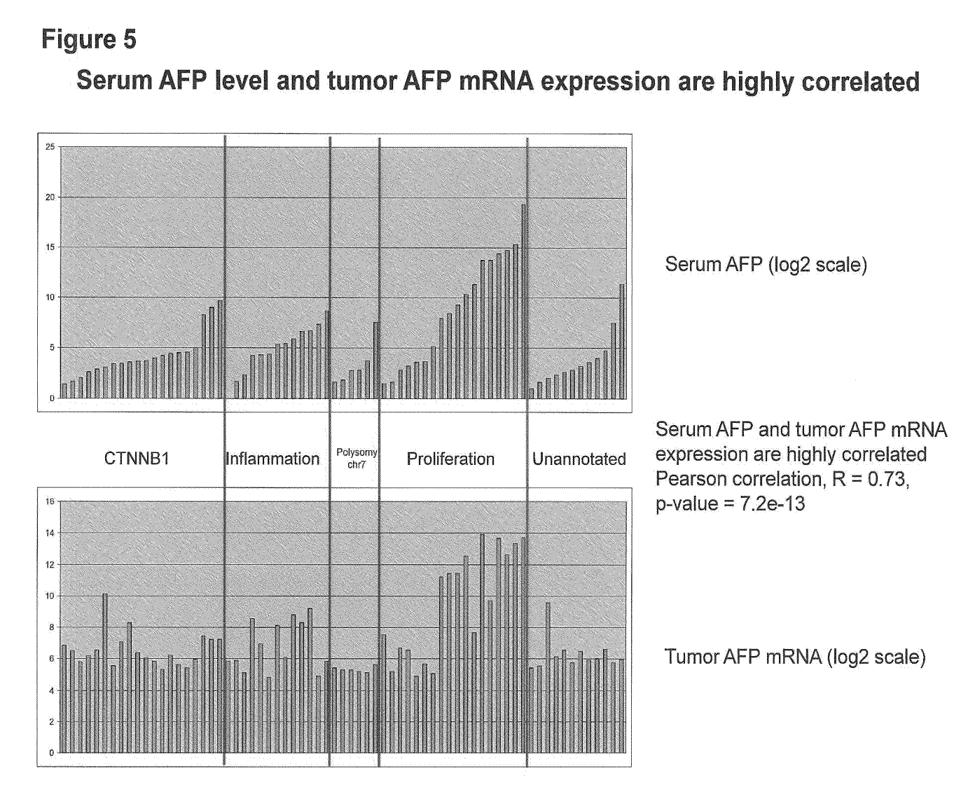

To test cell sensitivity of HCC cells to the IGF-1R kinase inhibitor OSI-906, twenty-one HCC cell lines were treated with different concentration of OSI-906, and cell proliferation was measured by cell-titer glo assays (FIG. 1). Seven HCC cell lines (HepG2, Hep3B, HUH-1, HUH-6, HUH-7, JHH-5, JHH-7) were very sensitive to OSI-906 (EC50<1 μM, and maximum inhibition >40%, FIG. 1, Table 1). AFP gene expression in 21 HCC cell lines was measured by quantitative RT-PCR (FIG. 2). All seven HCC cell lines sensitive to OSI-906 treatment expressed the highest levels of AFP. Correlation analyses indicated that AFP gene expression is highly correlated with HCC cell sensitivity to OSI-906 with correlation coefficient of 0.91 (p-value <0.0001) (FIG. 3). The growth media were collected from all 7 HCC cell lines sensitive to OSI-906, and secreted AFP concentration in the media was measured by ELISA (FIG. 4). All 7 HCC cells with high AFP expression also had the highest amount of secreted AFP protein in the growth media, and both measurements are highly correlated (correlation coefficient of 0.68, p-value=0.01). In HCC, patients that have tumors with high AFP expression and high serum AFP are enriched in the Proliferation subgroup, with 70% (16/23) of tumors expressing high levels of AFP in this subgroup, and only 16% (11/68) of all other tumors expressing high levels of AFP (FIG. 5). Consistent with data from HCC cell line studies, AFP expression and serum (secreted) AFP protein are also highly correlated in HCC tumors (correlation coefficient of 0.73, and p-value <0.001).

TABLE 2
Correlation between IGF axis components and sensitivity to OSI-906.
Total RNA was isolated from each HCC cell line and quantitative
RT-PCR was performed to measure expression levels of IGF-1, IGF-2, IR,
IGF-1R, and IGFBP1 to 7. Mean-centered deltaCT value of each
gene within the 21 HCC cell lines and EC50 from proliferation assays
with OSI-906 treatment was used for Pearson correlation analyses and
p-value calculation.

| | Pearson | |
|---|---|---|
| Gene | Correlation | p-value |
| IR | 0.498 | 0.022 |
| IGFBP3 | −0.473 | 0.030 |
| IGF2 | 0.461 | 0.036 |
| IGFBP7 | −0.435 | 0.049 |
| IGFBP2 | 0.401 | 0.080 |
| IGFBP4 | −0.386 | 0.084 |
| IGF1R | 0.373 | 0.096 |
| IGFBP1 | 0.337 | 0.135 |
| IGFBP6 | −0.331 | 0.142 |
| IGF-1 | −0.051 | 0.836 |
| IGFBP5 | −0.021 | 0.926 |

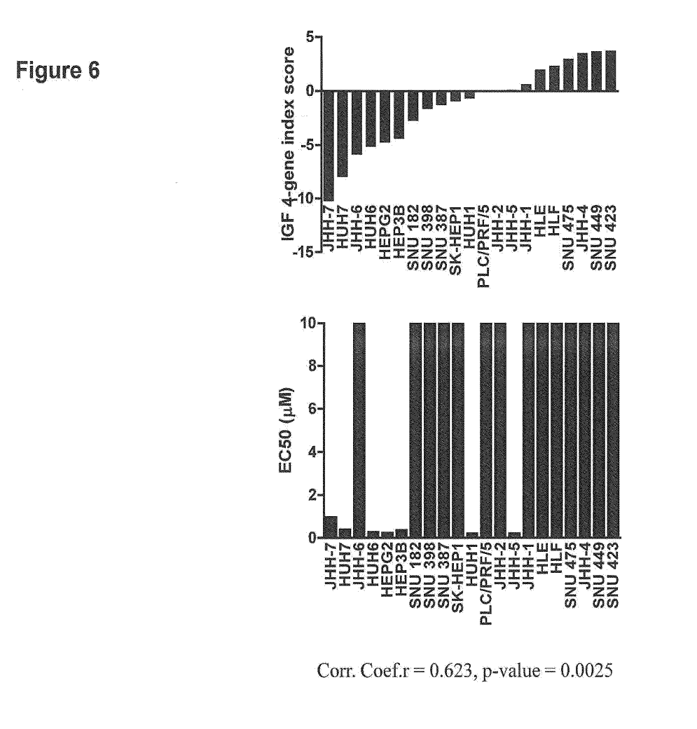

Gene expression levels of IGF ligands (IGF-1, IGF-2), receptors (IGF-1R, IR) and IGFBP1 to 7 were tested and their correlations with HCC cell sensitivity to OSI-906 were analyzed. Expressions of four genes (IGF-2, IR, IGFBP3, IGFBP7) correlated with OSI-906 sensitivity significantly (r>0.4, p value <0.05) (Table 2). An IGF axis index was calculated with these four genes, and the 4-gene index score significantly correlated with OSI-906 sensitivity (r=0.62, p=0.003; FIG. 6), which is considerably better than each individual gene of the signature.

Our finding indicates that total AFP expression is unexpectedly highly predictive of OSI-906 sensitivity in HCC cells. This is the first biomarker for IGF-1R sensitivity in HCC which is both highly predictive of efficacy and easily measurable in patient serum. Both data from in vitro HCC cell line studies and HCC tumors indicate AFP expression is highly correlated with secreted and serum AFP concentration. AFP expression being highly predictive of OSI-906 sensitivity is an unexpected finding because the functions of AFP proteins are still unclear and there seems no apparent direct connection between AFP and IGF-1R signaling pathways. HCC tumors with high AFP expression tend to have serum AFP concentrations of over 100 ng/ml, which could thus be used as a cut-off value for clinical investigations of small molecule inhibitors of IGF-1R kinase, such as OSI-906.

The data presented herein shows for the first time that an IGF index comprising the four genes IGF-2, INSR, IGFBP3 and IGFBP7 can predict sensitivity to OSI-906 in HCC cells. It is also significant that the individual genes IGFBP3, INSR, IGF2, and IGFBP7 each also have predictive value.

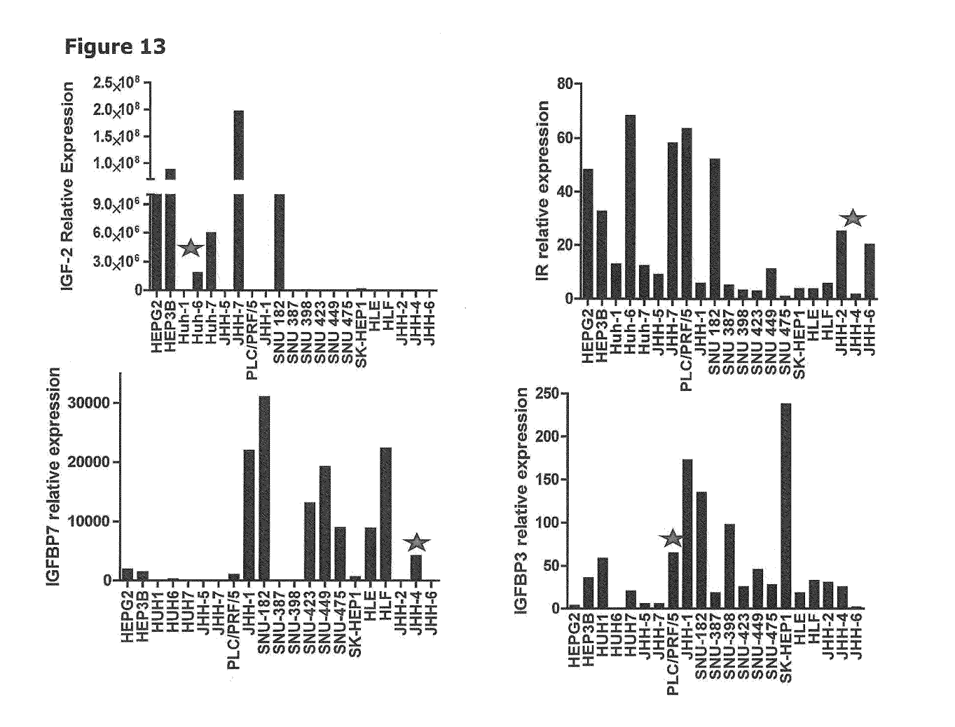

HCC cell lines described herein, or cells with similar levels, may be used to define a threshold between high and low gene expression when analyzing patient HCC tumor samples for gene expression levels in order to predict sensitivity to an IGF-1R kinase inhibitor (see FIG. 13). For IGF-2, a reference cell line could be HUH-6, wherein 5/6 cell lines with IGF2 expression equal or higher than HUH-6 are sensitive to OSI-906. For IGFBP7, a reference cell line could be JHH-4. None of the cell lines with IGFBP7 expression equal or higher than JHH-4 are sensitive to OSI-906. For IR, a reference cell line could be JHH-2, wherein 5/6 cell lines with IR expression equal or higher than JHH-2 are sensitive to OSI-906. For IGFBP3, a reference cell line could be PLC/PRF/5. None of the cell lines with IGFBP3 expression equal or higher than PLC/PRF/5 are sensitive to OSI-906.

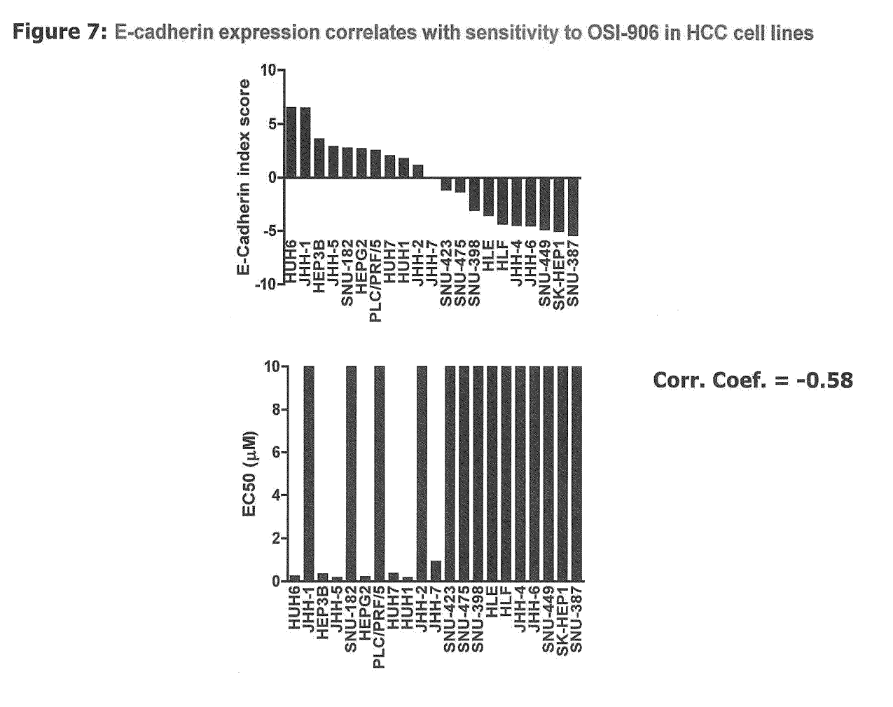

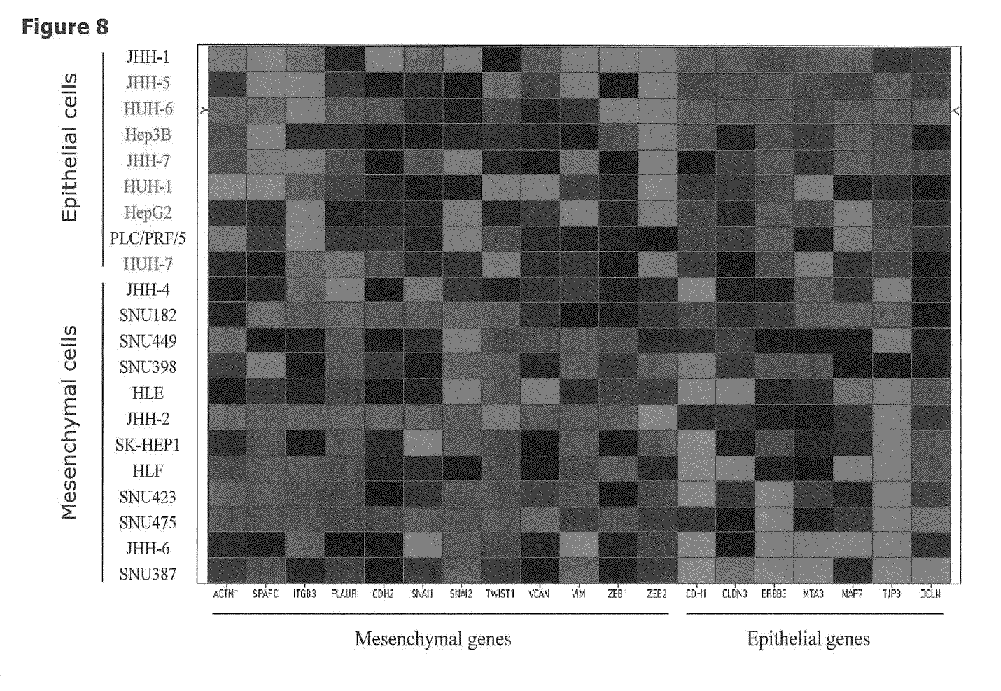

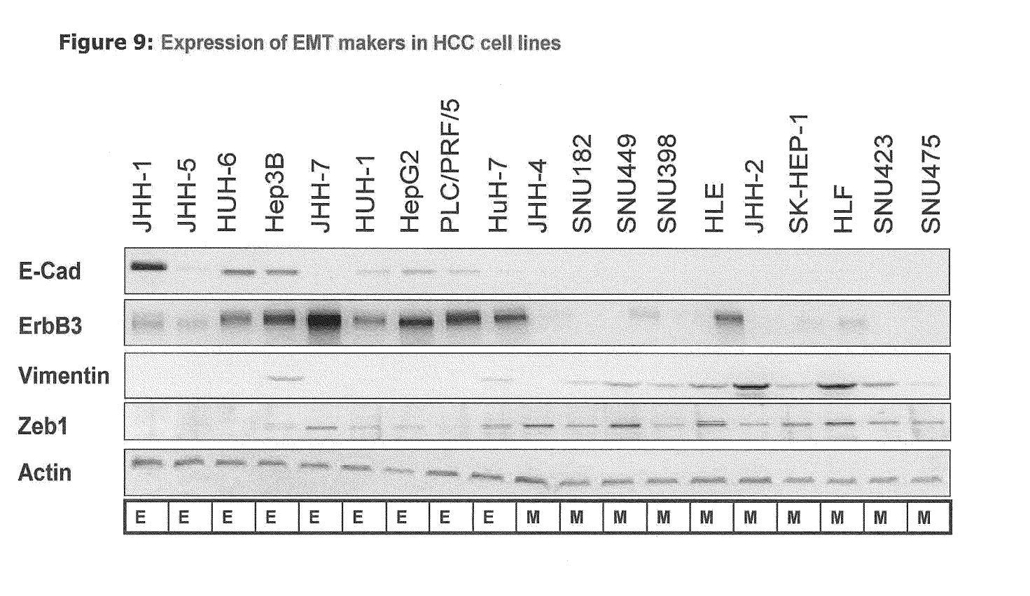

EMT status predicts sensitivity to OSI-906 in HCC cells. E-cadherin is a standard epithelial biomarkers, and its expression was found to correlate with sensitivity to OSI-906 in HCC tumor cells (correlation coefficient is −0.58; FIG. 7). In a similar experiment, ErbB3 expression was found to correlate with sensitivity to OSI-906 in HCC tumor cells (correlation coefficient −0.70). The epithelial-mesenchymal transition status in HCC tumor cells was also investigated using the expression of 12 gene transcripts associated with mesenchymal-like cells and 7 gene transcripts associated with epithelial cells (FIG. 8). Heatmap analysis indicated that nine cells expressed higher levels of epithelial genes and lower levels of mesenchymal genes, while the other twelve cell lines showed the opposite pattern. Analyses of protein markers were also used to establish EMT status of HCC cell lines. Protein expression of two epithelial markers (E-cadherin, ErbB3) and two mesenchymal markers (Vimentin and Zebl) was investigated (FIG. 9). Consistent with gene expression analysis, nine cell lines showed high levels of E-cadherin and ErbB3 proteins and low levels of Vimentin and Zebl, indicating that these nine cell lines are epithelial cells. A majority of epithelial cells (7 out of 9) are sensitive to OSI-906, indicating that EMT status can predict sensitivity to OSI-906 in HCC.

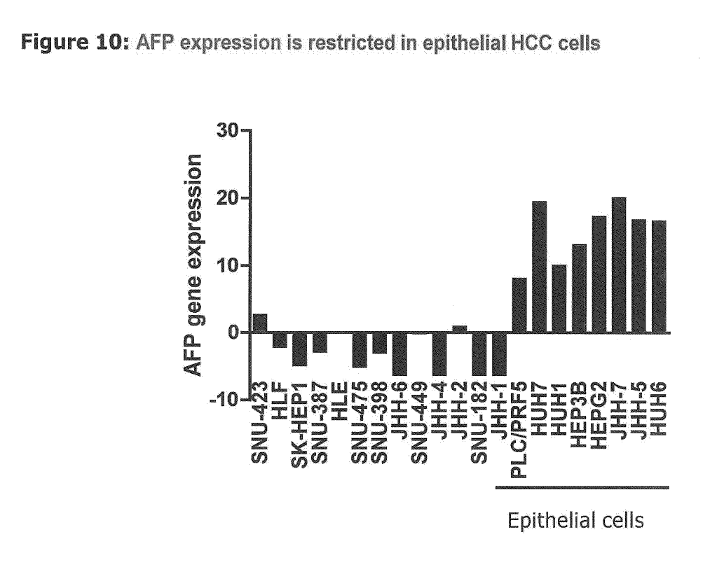

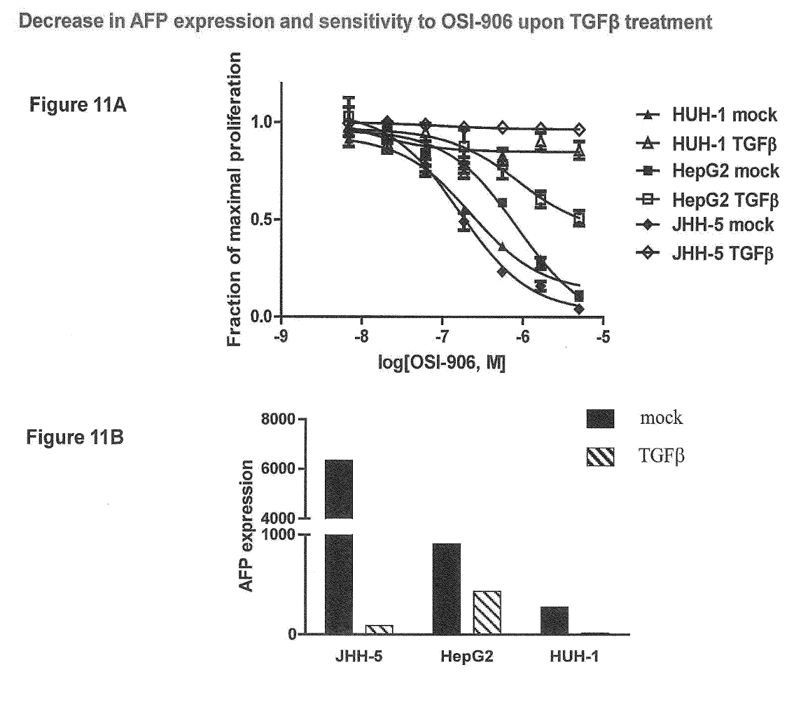

AFP expression is restricted to epithelial HCC cells (FIG. 10), suggesting that AFP is an epithelial marker in hepatocellular carcinoma cells. TGFβ is commonly used to induce a transition from an epithelial to a mesenchymal-like phenotype. Consistent with the finding that both EMT and AFP are determinants of sensitivity to OSI-906 in HCC, TGFβ treatment decreased AFP expression and also HCC cell sensitivity to OSI-906 (FIG. 11). TGFβ treatment also decreased expression of the epithelial biomarker protein ErbB3 in JHH5, HepG2, and HUH1 HCC cells (data not shown).

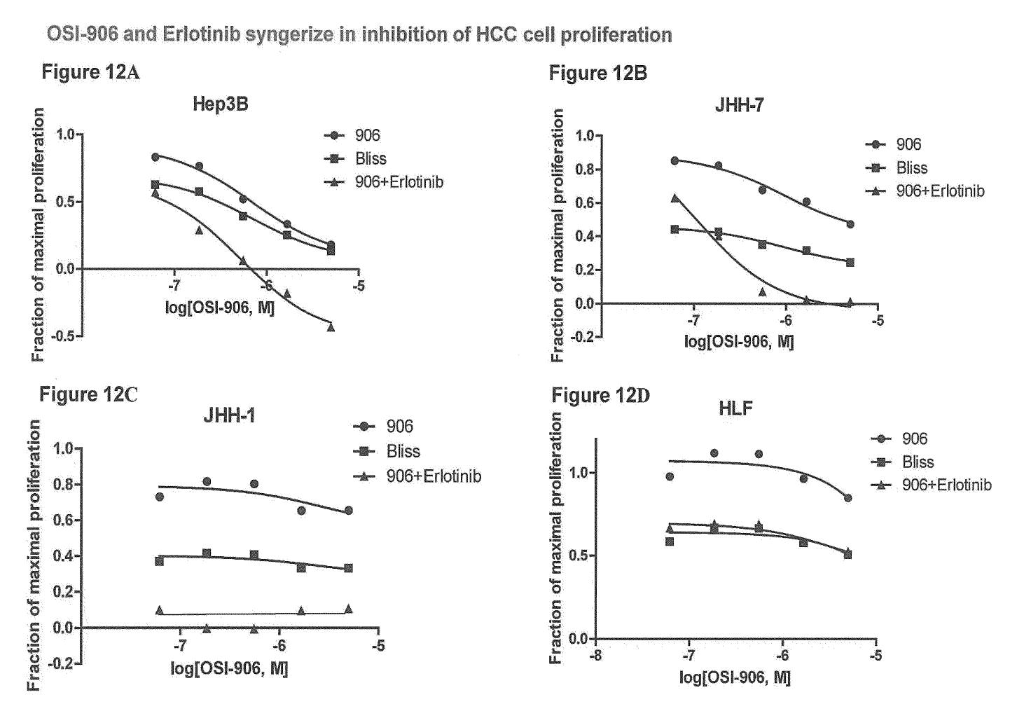

Epithelial HCC cells (Hep3B, JHH-1 and JHH-7) and mesenchymal-like HCC cells (HLF) were each treated with both OSI-906 and erlotinib to investigate the HCC cell response to this combination treatment (FIG. 12). The combination of OSI-906 and erlotinib showed a synergistic effect in only epithelial HCC cells (e.g. Hep3B, JHH-7), and not in mesenchymal-like HCC cells. This indicates that EMT status predicts responsiveness to the combination of OSI-906 and erlotinib in HCC. This data also demonstrates that OSI-906 and erlotinib show a synergistic effect in HCC cells that express high AFP levels (e.g. Hep3B, JHH-7; see FIGS. 3, 12), and not in those that express low AFP levels (e.g. HLF).

In summary, the various biomarkers disclosed herein (i.e. AFP, EMT, 4-gene index and individual genes thereof) will enable the creation of new diagnostic methods for predicting the effect of IGF-1R kinase inhibitors on patients with HCC, and will assist physicians in effective patient selection for treatment with these compounds.

Abbreviations

AFP, alpha-fetoprotein; EGF, epidermal growth factor; EMT, epithelial to mesenchymal transition; NSCLC, non-small cell lung carcinoma; SCC, squamous cell carcinoma; HNSCC, head and neck squamous cell carcinoma; CRC, colorectal cancer; MBC, metastatic breast cancer; EGFR, epidermal growth factor receptor; ErbB3, “v-erb-b2 erythroblastic leukemia viral oncogene homolog 3”, also known as HER-3; pHER3, phosphorylated HER3; Erk kinase, Extracellular signal-regulated protein kinase, also known as mitogen-activated protein kinase; CDH1, E-Cadherin gene; pErk, phosphorylated Erk; Brk, Breast tumor kinase (also known as protein tyrosine kinase 6 (PTK6)); LC, liquid chromatography; MS, mass spectrometry; IGF-1, insulin-like growth factor-1; IGF-2, insulin-like growth factor-2; INSR or IR, insulin receptor; IGF-1R or IGFR, insulin-like growth factor-1 receptor; TGFα, transforming growth factor alpha; HB-EGF, heparin-binding epidermal growth factor; LPA, lysophosphatidic acid; TGFα, transforming growth factor alpha; IC₅₀, half maximal inhibitory concentration; RT, room temperature; pY, phosphotyrosine; pPROTEIN, phospho-PROTEIN, “PROTEIN” can be any protein that can be phosphorylated, e.g. EGFR, ERK, HER3, S6 etc; wt, wild-type; PI3K, phosphatidyl inositol-3 kinase; GAPDH, Glyceraldehyde 3-phosphate dehydrogenase; TKI, Tyrosine Kinase Inhibitor; PMID, PubMed Unique Identifier; NCBI, National Center for Biotechnology Information; NCl, National Cancer Institute; MSKCC, Memorial Sloan Kettering Cancer Center; ECACC, European Collection of Cell Cultures; ATCC, American Type Culture Collection; K-RAS, v-Ki-ras2 Kirsten rat sarcoma viral oncogene homolog; B-RAF, v-raf murine sarcoma viral oncogene homolog B1; PIK3CA, phosphoinositide-3-kinase, catalytic, alpha polypeptide.

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

1-92. (canceled)

93. A method of identifying patients with hepatocellular carcinoma (HCC) who are most likely to benefit from treatment with an IGF-1R kinase inhibitor, comprising:
determining whether the tumor cells in a sample of a patient's HCC tumor express a high level of AFP; and
identifying the patient as one most likely to benefit from treatment with an IGF-1R kinase inhibitor if the tumor cells express a high level of AFP.

94. The method of claim 93, wherein a high level is a level above a defined threshold as determined by a threshold determination analysis.

95. The method of claim 94, wherein the threshold determination analysis comprises a receiver operator characteristic curve analysis.

96. The method of claim 93 wherein a high level is a level equal to or greater than a level found in a reference HCC tumor cell that has high sensitivity to growth inhibition by an IGF-1R kinase inhibitor.

97. The method of claim 93, wherein the IGF-1R kinase inhibitor comprises OSI-906.

98. A method for treating hepatocellular carcinoma in a patient, comprising administering to said patient a therapeutically effective amount of an IGF-1R kinase inhibitor if the patient has been diagnosed to be potentially responsive to an IGF-1R kinase inhibitor by a determination that the tumor cells of the patient express a high level of AFP.

99. The method of claim 98, wherein the IGF-1R kinase inhibitor comprises OSI-906.

100. A method of identifying patients with hepatocellular carcinoma (HCC) who are most likely to benefit from treatment with an IGF-1R kinase inhibitor, comprising:
determining the level of AFP protein in a serum sample from the patient; and
identifying the patient as likely to benefit from treatment with an IGF-1R kinase inhibitor if the serum contains a high level of AFP.

101. The method of any of claim 100, wherein a high level is a level above a defined threshold as determined by a threshold determination analysis.

102. The method of claim 101, wherein the threshold determination analysis comprises a receiver operator characteristic curve analysis.

103. The method of claim 100, wherein a high level is a level equal to or greater than a level found in a reference serum sample from a patient who's tumor has high sensitivity to growth inhibition by an IGF-1R kinase inhibitor.

104. The method of claim 100, wherein the IGF-1R kinase inhibitor comprises OSI-906.

105. A method for treating hepatocellular carcinoma in a patient, comprising administering to said patient a therapeutically effective amount of an IGF-1R kinase inhibitor if the patient has been diagnosed to be potentially responsive to an IGF-1R kinase inhibitor by a determination that the serum of the patient contains a high level of AFP.

106. The method of claim 105, wherein the IGF-1R kinase inhibitor comprises OSI-906.

107. A method of identifying patients with hepatocellular carcinoma (HCC) who are most likely to benefit from treatment with an IGF-1R kinase inhibitor in combination with another anticancer agent, comprising:
determining whether the tumor cells in a sample of a patient's HCC tumor express a high level of AFP; and
identifying the patient as one most likely to benefit from treatment with an IGF-1R kinase inhibitor in combination with the other anticancer agent if the tumor cells express a high level of AFP.

108. The method of claim 107, wherein a high level is a level above a defined threshold as determined by a threshold determination analysis.

109. The method of claim 108, wherein the threshold determination analysis comprises a receiver operator characteristic curve analysis.

110. The method of claim 107, wherein a high level is a level equal to or greater than a level found in a reference HCC tumor cell that has high sensitivity to growth inhibition by an IGF-1R kinase inhibitor.

111. The method of claim 107, wherein the IGF-1R kinase inhibitor comprises OSI-906.

112. The method of claim 107, wherein the other anticancer agent is an EGFR kinase inhibitor.

113. The method of claim 112, wherein the EGFR kinase inhibitor comprises erlotinib.

114. A method for treating hepatocellular carcinoma in a patient, comprising administering to said patient a therapeutically effective amount of an IGF-1R kinase inhibitor in combination with another anticancer agent if the patient has been diagnosed to be potentially responsive to an IGF-1R kinase inhibitor in combination with the other anticancer agent by a determination that the tumor cells of the patient express a high level of AFP.

115. The method of claim 114, wherein the small molecule IGF-1R kinase inhibitor comprises OSI-906.

116. The method of claim 114, wherein the other anticancer agent is an EGFR kinase inhibitor.

117. The method of claim 116, wherein the EGFR kinase inhibitor comprises erlotinib.

118. A method of identifying patients with hepatocellular carcinoma (HCC) who are most likely to benefit from treatment with an IGF-1R kinase inhibitor in combination with another anticancer agent, comprising:
determining the level of AFP protein in a serum sample from the patient;
identifying the patient as likely to benefit from treatment with an IGF-1R kinase inhibitor if the serum contains a high level of AFP.

119. The method of claim 118, wherein a high level is a level above a defined threshold as determined by a threshold determination analysis.

120. The method of claim 119, wherein the threshold determination analysis comprises a receiver operator characteristic curve analysis.

121. The method of claim 118 wherein a high level is a level equal to or greater than a level found in a reference serum sample from a patient who's tumor has high sensitivity to growth inhibition by an IGF-1R kinase inhibitor.

122. The method of claim 118, wherein the IGF-1R kinase inhibitor comprises OSI-906.

123. The method of claim 118, wherein the other anticancer agent is an EGFR kinase inhibitor.

124. The method of claim 123, wherein the EGFR kinase inhibitor comprises erlotinib.

125. A method for treating hepatocellular carcinoma in a patient, comprising administering to said patient a therapeutically effective amount of an IGF-1R kinase inhibitor in combination with another anticancer agent if the patient has been diagnosed to be potentially responsive to an IGF-1R kinase inhibitor in combination with another anticancer agent by a determination that the serum of the patient contains a high level of AFP.

126. The method of claim 125, wherein the small molecule IGF-1R kinase inhibitor comprises OSI-906.

127. The method of claim 125, wherein the other anticancer agent is an EGFR kinase inhibitor.

128. The method of claim 127, wherein the EGFR kinase inhibitor comprises erlotinib.

129. A method of identifying patients with a tumor who are most likely to benefit from treatment with an IGF-1R kinase inhibitor, comprising:
measuring in a sample of the tumor from the patient the relative expression level of each gene of a 4-gene signature (4GS), wherein the 4GS consists essentially of the following genes:
INSR, IGF2, IGFBP3 and IGFBP7;
calculating a 4GS index score for said tumor cells according to the equation:
$$4 GS index score = \frac{1}{n} {\sum }_{i ∈ IGF} {g}_{i} \cdot r ,$$
wherein: IGF=the genes IGF2, INSR, IGFBP3, and IGFBP7; n=number of genes in the gene signature=4; gᵢ=median centered expression value of gene i; and r=+1 for IGF2 and INSR, and r=−1 for IGFBP3 and IGFBP7;
determining if said 4GS index score is more similar to a 4GS index score from a reference tumor cell that is sensitive to growth inhibition by an IGF-1R kinase inhibitor or an 4GS index score from a reference tumor cell that is resistant to growth inhibition by an IGF-1R kinase inhibitor, and
identifying the patient as one likely to benefit from treatment with an IGF-1R kinase inhibitor if their tumor cells have a 4GS index score that is more similar to a 4GS index score from a reference tumor cell that is sensitive to growth inhibition by an IGF-1R kinase inhibitor.

130. The method of claim 129, wherein the tumor is a hepatocellular carcinoma (HCC).

131. The method of claim 129, wherein the sample of tumor cells is derived from a tumor biopsy.

132. The method of claim 129, wherein the sample of tumor cells is derived from a blood sample containing circulating tumor cells.

133. The method of claim 129, wherein the IGF-1R kinase inhibitor comprises OSI-906.

134. A method of treatment of a patient with cancer, comprising:
administering to said patient a therapeutically effective amount of an IGF-1R kinase inhibitor if the patient is predicted to be responsive to an IGF-1R kinase inhibitor using the method of claim 129.

135. The method of claim 134, wherein the IGF-1R kinase inhibitor comprises OSI-906.

136. A method of identifying a tumor as likely to be responsive or non-responsive to treatment with an IGF-1R kinase inhibitor, comprising:
measuring in a sample of the tumor cells the relative expression level of each gene of a 4-gene signature (4GS), wherein the 4GS consists essentially of the following genes: INSR, IGF2, IGFBP3 and IGFBP7;
calculating a 4GS index score for said tumor cells according to the equation:
$$4 GS index score = \frac{1}{n} {\sum }_{i ∈ IGF} {g}_{i} \cdot r ,$$
wherein: IGF=the genes IGF2, INSR, IGFBP3, and IGFBP7; n=number of genes in the gene signature=4; gᵢ=median centered expression value of gene i; and r=+1 for IGF2 and INSR, and r=−1 for IGFBP3 and IGFBP7; and
determining if the 4GS index score is above a defined threshold that indicates that the tumor is likely to be responsive to an IGF-1R kinase inhibitor, or below said threshold and thus likely to be non-responsive to an IGF-1R kinase inhibitor.

137. The method of claim 136, wherein the tumor is a hepatocellular carcinoma (HCC).

138. The method of claim 136, wherein the sample of tumor cells is derived from a tumor biopsy.

139. The method of claim 136, wherein the sample of tumor cells is derived from a blood sample containing circulating tumor cells.

140. The method of claim 136, wherein the small molecule IGF-1R kinase inhibitor comprises OSI-906.

141. A method of treatment of a patient with cancer, comprising:
administering to said patient a therapeutically effective amount of an IGF-1R kinase inhibitor if the patient is predicted to be responsive to an IGF-1R kinase inhibitor using the method of claim 136.

142. The method of claim 141, wherein the IGF-1R kinase inhibitor comprises OSI-906.
